(19) United States
(12) Patent Application Publication
(10) Pub. No.: US 2024/0336975 A1
(43) Pub. Date: Oct. 10, 2024

(54) Biomarkers Predicting Response of Breast Cancer to Immunotherapy

(71) Applicants: VIB VZW, Gent (BE); Katholieke Universiteit Leuven, Leuven (BE)

(72) Inventors: Diether Lambrechts, Kessel-Lo (BE); Ayse Bassez, Gent (BE); Hanne Vos, Leuven (BE); Ann Smeets, Leuven (BE)

(21) Appl. No.: 18/579,415

(22) PCT Filed: Jul. 13, 2022
(86) PCT No.: PCT/EP2022/069583

§ 371 Date: Jan. 15, 2024

(30) Foreign Application Priority Data
Jul. 15, 2021 (EP) 21185934.3
Mar. 30, 2022 (EP) 22165625.9

(51) Int. Cl. C12Q 1/6886

(52) U.S. Cl. CPC C12Q 1/6886; C12Q 2600/106; C12Q 2600/158

(57) ABSTRACT

The invention relates to methods of breast tumor analysis relying on the detection of expression levels of specific genes. Such methods find application in, amongst other, predicting the response of a breast cancer patient to immunotherapy or to immunogenic therapy, and in following up such responses. The expression levels of the genes can thus be used in determining which breast cancer patients are most likely to respond to immunotherapy or immunogenic therapy. The genes serving as biomarkers are individually highly performant in predicting the response of a breast tumor to immunotherapy or to immunogenic therapy, but are weak in predicting the response of melanoma to immunotherapy or to immunogenic therapy. Diagnostic kits are likewise part of the invention.

### CROSS REFERENCE TO RELATED APPLICATIONS

This application is a national phase entry under 35 U.S.C. § 371 of International Patent Application PCT/EP2022/069583, filed Jul. 13, 2022, designating the United States of America and published in English as International Patent Publication WO 2023/285521 on Jan. 19, 2023, which claims the benefit under Article 8 of the Patent Cooperation Treaty to European Patent Application Serial No. 21185934.3, filed Jul. 15, 2021, and European Patent Application Serial No. 22165625.9, filed Mar. 30, 2022, the entireties of which are hereby incorporated by reference.

### FIELD OF THE INVENTION

The invention relates to methods of breast tumor analysis relying on the detection of expression levels of specific genes. Such methods find application in, amongst other, predicting the response of a breast cancer patient to immunotherapy or to immunogenic therapy, and in following up such responses. The expression levels of the genes can thus be used in determining which breast cancer patients are most likely to respond to immunotherapy or immunogenic therapy. The genes serving as biomarkers are individually highly performant in predicting the response of a breast tumor to immunotherapy or to immunogenic therapy, but are weak in predicting the response of melanoma to immunotherapy or to immunogenic therapy. Diagnostic kits are likewise part of the invention.

### BACKGROUND

As of its coming to existence, a tumor is creating and/or forced to create its own specific “ecosystem” within the surrounding healthy tissue. Many factors and processes are decisive over whether or not the single tumor cell will be able to create, and support, its ecosystem and, therewith, growth. In an attempt to create a simplifying overview, Blank et al. 2016 (Science 352: 658-660) designed a visually appealing “cancer immunogram” in which currently known factors and processes influencing tumor growth/survival are grouped in seven classes of parameters. For each individual patient/tumor, the status of the seven classes of parameters can be plotted, the resulting plot giving insight in treatment options. Somewhat similar to the cancer immunogram, Charoentong et al. 2017 (Cell Reports 18:248-262) designed an immunophenogram/immunophenoscore which provides an as yet to be further validated tool for predicting response of a tumor to checkpoint blockade therapy (as the majority of cancer patients do not respond to such therapy). Such tools strongly underscore the need to expand knowledge on the status of a tumor or cancer as this, besides potentially leading to identification of new therapeutic targets, aids in deciding on the optimal (available) treatment for each individual tumor.

Triple-negative breast cancer (TNBC) comprises ˜15% of all breast cancer (BC) patients and represents a heterogeneous group of tumors associated with poor outcome (The Cancer Genome Atlas Network. Comprehensive molecular portraits of human breast tumours. Nature 490:61-70 (2012)). Of all BC subtypes, TNBC exhibits the highest number of tumor-infiltrating lymphocytes (TILs) (Loi et al. 2013, J Clin Oncol 31:860-867; Denkert et al. 2018, Lancet Oncol 19:40-50), suggesting that TNBC could benefit from immune checkpoint blockade (ICB). The neutralizing PD-L1 antibody atezolizumab improves progression-free and overall survival when combined with nab-paclitaxel as first-line treatment in PD-L1+ metastatic TNBC (Schmid et al. 2020, Lancet Oncol 21:44-59). Anti-PD-L1 alone was also superior to maintenance chemotherapy in metastatic TNBC that was not progressive after six to eight cycles of chemotherapy (Bachelot et al. 2021, Nat Med 27:250-255). Moreover, interim analysis of KEYNOTE-522, investigating the addition of an anti-PD1 antibody to neoadjuvant platinum-containing chemotherapy in previously untreated early TNBC, revealed an improvement in pathological complete response (pCR) rate and event-free survival (Schmid et al. 2020, New Engl J Med 382:810-821). ICB is also being explored as a neoadjuvant treatment for other BC subtypes (NCT03725059 or KEYNOTE-756 in estrogen receptor-positive (ER+) and human epidermal growth factor receptor 2-negative BC (HER2− BC). This suggests that neoadjuvant ICB will soon become part of the standard of care for BC treatment. However, not all BC patients respond to neoadjuvant ICB. An outstanding question is therefore to identify which underlying mechanisms and associated markers determine treatment response. So far, TIL scores and tumor PD-L1 expression have been proposed to predict clinical outcome (Schmid et al. 2020, Ann Oncol 31:569-581; Loibl et al. 2019, Ann Oncol 30:1279-1288), but their efficacies as predictive markers are still unclear. Indeed, TILs represent a heterogeneous population of cells with respect to cell type composition, gene expression and functional properties, and also differ between BC subtypes (Denkert et al. 2018, Lancet Oncol 19:40-50; Chen & Mellman 2017, Nature 541:321-330). It is also not straightforward to delineate how TILs affect treatment outcome, as in the neoadjuvant setting ICB is combined with chemotherapy and the response to neoadjuvant chemotherapy itself depends on TILs (Denkert et al. 2018, Lancet Oncol 19:40-50). In several other cancers, such as melanoma or lung cancer, clonal expansion of T cells underlies the treatment response to ICB (Amaria et al. 2018, Nat Med 24:1649-1654; Tumeh et al. 2014, Nature 515:568-571; Forde et al. 2018, New Engl J Med 378:1976-1986). Single-cell characterization of pre- and on-treatment biopsies has provided important insights into the patterns of T cell expansion and its underlying mechanisms (Sade-Feldman et al. 2018, Cell 175:998-1013; Yost et al. 2019, Nat Med 25:1251-1259). However, these studies have so far only been performed on easy-to-biopsy cancer types, such as melanoma or basal/squamous cell skin carcinoma, profiling few patients or focusing exclusively on CD45-positive immune cells. Others have characterized treatment-naive BC microenvironments at single-cell resolution (Wagner et al. 2019, Cell 177:1330-1345; Jackson et al. 2020, Nature 578:615-620; Savas et al. 2018, Nat Med 24:986-993). General intratumoral changes at the single-cell level in BC patients receiving ICB therapy have been reported (Bassez et al. 2021, Nat Med 27:820-832).

The involvement of B-cells in the ICB treatment outcome was reported in US2020/123258A1. Genes informative of immune cell types involved in the response of cancer/a cancer patient to different immune therapies treatment were reported (WO2018/209324, WO2020/205644, US2019/0295720, WO2016/109546). Some biomarkers for BC or other cancers were related to immunotherapies, immune evasion, or prediction of tolerance (US 2017/260594; WO 2020/109570; WO 2012/129488; US 2019/0112671; US 2019/369098). Biomarkers clearly and unequivocally informative of response of BC patients to immune therapy such as ICB therapy and sufficiently informative when starting from bulk sequence data have not yet been identified.

### SUMMARY OF THE INVENTION

The current invention relates to methods of selecting a subject having breast cancer for treatment with an immunotherapy or with an immunogenic therapy, such methods comprising:
quantifying in a sample obtained from the subject prior to start, at start, or early after start of the immunotherapy or of the immunogenic therapy the expression level of at least one gene selected from the genes IGHG1, IGHG2, IGHG3, IGLC2, IGKC, HMGB2, MZB1, SEC11C, BATF, CXCL13, CXCR6, GZMB, PDCD1, TIGIT or TNFRSF18;
selecting a subject having breast cancer for treatment with the immunotherapy or with the immunogenic therapy, when the expression level quantified for the selected gene is within a pre-determined range of expression level values of the gene wherein the pre-determined range of expression levels is indicative of a positive outcome of the immunotherapy or of the immunogenic therapy.

In one embodiment, the at least one gene is selected from the genes IGHG1, IGHG2, IGHG3, IGLC2, and IGKC. In an alternative embodiment, the at least one gene is selected from the genes IGHG1, IGHG2, IGHG3, IGLC2, IGKC, HMGB2, MZB1, SEC11C, BATF, CXCR6, PDCD1, TIGIT or TNFRSF18.

The methods of the invention may further be comprising:
quantifying one or more of the Gini-index of T-cell receptor clonotype distribution, the relative frequency of exhausted T-cells, the relative frequency of exhausted CD4+ T-cells, the relative frequency of exhausted CD8+ T-cells, the T-cell receptor richness, or the relative frequency of the C7_CX3CR1 subtype of T-cell inhibitory macrophages;
wherein high values within a pre-determined range of values of the Gini-index of T-cell receptor clonotype distribution, the relative frequency of exhausted T-cells, the relative frequency of exhausted CD4+ T-cells, and/or of the relative frequency of exhausted CD8+ T-cells are further indicative of a positive outcome of the immunotherapy or of the immunogenic therapy, and low values within a pre-determined range of values of the T-cell receptor richness, and/or of the relative frequency of the C7_CX3CR1 subtype of T-cell inhibitory macrophages are further indicative of a positive outcome of the immunotherapy or of the immunogenic therapy.

In any of the methods of the invention, the quantification of the gene expression level is determined by transcriptome analysis and/or is determined by proteome analysis. In one embodiment, the transcriptome analysis is analysis of the bulk transcriptome.

Any of the methods of the invention may further include detecting the status of one or more further diagnostic markers or biomarkers selected from immune checkpoint gene expression, markers of tumor mutational burden, T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES), expression of retrotransposons, infiltration of immune cells in the tumor of a subject having breast cancer, wherein the status of the one or more further diagnostic markers or biomarkers is further indicative of the positive outcome of the immunotherapy or of the immunogenic therapy.

In one embodiment to these methods, the immunotherapy or immunogenic therapy is a therapy comprising an immune checkpoint blocker.

In a further embodiment to these methods, at least one data collecting step or at least one analysis step is performed by a computer system or via a computer program product. The invention therefore likewise relates to computer products comprising a computer readable medium storing instructions for operating a computer system to perform at least one data collecting step or at least one analysis step of a method according to any one of the methods of the invention.

The invention further relates to immunotherapeutic or immunogenic agents for use in treating a subject having breast cancer, for use in inhibiting breast cancer progression or relapse, or for use in inhibiting breast cancer metastasis, comprising:
quantifying in a sample obtained from the subject prior to start, at start, or early after start of therapy with the immunotherapeutic or immunogenic agent the expression level of at least one gene selected from the genes IGHG1, IGHG2, IGHG3, IGLC2, IGKC, HMGB2, MZB1, SEC11C, BATF, CXCL13, CXCR6, GZMB, PDCD1, TIGIT or TNFRSF18;
subject having breast cancer for treatment with or for continued treatment with the immunotherapeutic or immunogenic agent, when the expression level quantified for the selected gene is within a pre-determined range of expression level values of the gene wherein the pre-determined range of expression levels is indicative of a positive outcome of the treatment of breast cancer, indicative of inhibition or relapse of breast cancer, or indicative of inhibition of breast cancer metastasis. In one embodiment, the therapeutic or immunogenic agent is comprising an immune checkpoint blocker.

The invention further relates to the use of a panel of genes in a method according to the invention, wherein the panel is comprising 1 to 15 genes selected from IGHG1, IGHG2, IGHG3, IGLC2, IGKC, HMGB2, MZB1, SEC11C, BATF, CXCL13, CXCR6, GZMB, PDCD1, TIGIT or TNFRSF18.

The invention also relates to diagnostic kits such as for use in a method of the invention, wherein the kit is comprising the tools to detect the expression level of at least one gene selected from IGHG1, IGHG2, IGHG3, IGLC2, IGKC, HMGB2, MZB1, SEC11C, BATF, CXCL13, CXCR6, GZMB, PDCD1, TIGIT or TNFRSF18.

In one embodiment, the diagnostic kit is including the tools for detecting the status of at most 500 markers.

### DESCRIPTION TO THE FIGURES

FIG. 1. Number of expanded clonotypes in Expanders (“E”; n=8) and Non-Expanders (“NE”; n=19) comparing ER+ (n=15; Es: n=3) versus triple-negative breast cancer (TNBC,n=12; Es: n=5). No significant difference was observed between ER+ Es and TNBC Es.

FIGS. 2A and 2B. Boxplots of the individual feature values of the 6-feature signature. P-values shown are calculated with a two-sided Mann-Whitney test. Box, median±interquartile rage; whiskers, 1.5× interquartile range. “E”: Expander; “NE”: Non-Expander. “PRE_EX”: T_{EX}-cells; “PRE_richness”: TCR richness; “PRE-Gini”: Gini index of T-cell clonotype distribution; “PRE_CD4_EX”: CD4 T_{EX} cells; “PRE_C7_CX3CR1”: C7_CX3CR1 macrophages; “PRE_CD8_EX”: CD8 T_{EX} cells.

FIGS. 3A-3H. Boxplots of the individual expression values of the 15 identified genes in the discovery cohort (left panels) and in the validation cohort (right panels). P-values shown are calculated with a two-sided Mann-Whitney test. Box, median±interquartile rage; whiskers, 1.5× interquartile range.

FIGS. 4A and 4B. Heatmap showing the expression of the indicated genes in the indicated cell types in scRNA-seq data obtained from the early breast cancer samples studied herein.

FIGS. 5A and 5B. Heatmap showing the expression of the indicated genes in the indicated major cell subtypes in scRNA-seq data obtained from the early breast cancer samples studied herein.

FIGS. 6A and 6B. Heatmap showing the expression of the indicated genes in each cell type in blood mononuclear cell (2 donors, 2 sites) data from ImmGen Single Cell Portal. Size of dots indicate percentage of cells expressing a certain gene.

FIG. 7. Features (as in FIGS. 2A and 2B) ranked according to their predictive potential (shrunken class centroids) to predict T-cell expansion in the PAM model, with T_{EX}-cells defined as the most predictive feature of the signature. T_{EX}-cells, experienced T-cells. “E”: expander score; “NE”: non-expander score.

FIG. 8. Six-feature (as in FIGS. 2A and 2B) combined signature scores by Bayesian regression fitted on discovery cohort. P-values shown are calculated with a two-sided Mann-Whitney test. Box, median±interquartile rage; whiskers, 1.5× interquartile range. DC: discovery cohort; VC: validation cohort; E: expanders; NE: non-expanders.

FIG. 9. Features (as in FIGS. 3A-3H) ranked according to their predictive potential (shrunken class centroids) to predict T-cell expansion in the PAM model. NE: non-expander; E: expander.

### DETAILED DESCRIPTION TO THE INVENTION

In work leading to the invention, and in view of the art lacking clear and unambiguous biomarkers predictive for response of breast cancer (BC) patients to checkpoint inhibitor immunotherapy, the inventors identified a set of biomarkers of which each individual biomarker is highly performant in predicting response of breast cancer (BC) patients to anti-PD1 (antibody blocking Programmed Cell Death 1). The inventors moreover concluded that the biomarkers predictive for response of breast cancer (BC) patients to anti-PD1 as identified herein were significantly less performant or powerful in predicting response of melanoma patients to anti-PD1 (with melanoma being the only cancer for which sufficient public data regarding anti-PD1 treatment are available). As such, the herein identified biomarkers share the same technical effect of being performant in the setting of predicting a positive response of breast cancer (BC) patients to immunotherapy. Further shared is the higher prognostic performance of the herein identified biomarkers in predicting a positive response of BC patients compared to melanoma patients to immunotherapy. The herein identified biomarkers do not form an arbitrary choice from prior long lists of genes as the herein identified biomarkers all were consistently selected for their individual high performance in predicting a positive response of breast cancer (BC) patients to immunotherapy, this in contrast to the low performance of the same biomarkers in predicting a positive response of melanoma patients to immunotherapy.

More in particular, selection of these biomarkers highly performant in predicting response of BC patients, but not of melanoma patients to anti-PD1, involved unbiased assessment of intratumoral changes in breast cancer (BC) patients receiving immune checkpoint blockade (ICB) therapy by accurately monitoring how anti-PD1 (antibody blocking Programmed Cell Death 1) affects immune cells and vice versa how pre-treatment immune cell status correlates with T-cell expansion.

Furthermore in particular, the selected biomarkers highly perform independent of whether the immunotherapy or immunogenic therapy is combined with chemotherapy.

Two different analyses were performed. The first independent analysis included the average expression of all genes in all pre-treatment cells per patient as input, therewith mimicking bulk-sequencing data.

Biomarkers correlating with T-cell expansion after 1 cycle of anti-PD1 treatment were identified and are described hereinafter. The second independent analysis included TCR measures (i.e., clonality, richness and Gini index) and relative frequencies of cell (sub)types at baseline per patient.

As described in the Examples, to identify the mechanisms underlying the response to ICB specifically in BC, 40 early-diagnosed breast cancer (BC) patients were treated with neoadjuvant anti-PD1 and intratumoral changes were monitored by subjecting matched pre- and on-treatment biopsies to single-cell transcriptome (scRNA-seq), T cell receptor (scTCR-seq) and combined transcriptome and proteome (CITE-seq) sequencing. Briefly, one cohort of patients with non-metastatic, treatment-naive primary invasive carcinoma of the breast was treated with one dose of pembrolizumab (anti-PD1) approximately 9±2 days before surgery. A second cohort of patients received neoadjuvant chemotherapy for 20-24 weeks, which was followed by pembrolizumab before surgery. In both cohorts, a tumor biopsy was collected immediately before anti-PD1 treatment (‘pre-treatment’), while another biopsy was collected during subsequent surgery (‘on-treatment’). Patients with different BC subtypes were included.

The invention therefore in one aspect relates to a number of methods focusing on identifying subjects having breast cancer that are likely to respond to immunotherapy or to immunogenic therapy.

Such methods include:
1) methods of selecting, identifying, choosing, or collecting (or, contrary, of refusing or rejecting) a subject or subjects having breast cancer for treatment with an immunotherapy or with an immunogenic therapy, comprising one or more steps of:
   quantifying in a sample obtained from the subject prior to start, at start, or early after start of the immunotherapy or of the immunogenic therapy the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18;
   electing, identifying, choosing, or collecting (or, contrary, of refusing or rejecting) a subject having breast cancer for treatment with the immunotherapy or with the immunogenic therapy, when the expression level quantified for the selected gene is within a pre-determined range of expression levels/expression level values/standard values of the gene wherein the pre-determined range of expression levels is indicative of/associated with positive outcome of the immunotherapy or of the immunogenic therapy; or
2) methods of selecting, identifying, choosing, or collecting a subject or subjects having breast cancer fit or proper for treatment with an immunotherapy or with an immunogenic therapy, comprising one or more steps of:
   quantifying in a sample obtained from the subject prior to start, at start, or early after start of the immunotherapy or of the immunogenic therapy the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18;
   selecting, identifying, choosing, or collecting a subject having breast cancer for treatment with the immunotherapy or with the immunogenic therapy, when the expression level quantified for the selected gene is within a pre-determined range of expression levels/expression level values/standard values of the gene wherein the pre-determined range of expression levels is indicative of/associated with positive outcome of the immunotherapy or of the immunogenic therapy; or
3) methods of determining eligibility, susceptibility, qualification, suitability or acceptability of a subject having breast cancer for treatment with an immunotherapy or with an immunogenic therapy, comprising one or more steps of:
   quantifying in a sample obtained from the subject prior to start, at start, or early after start of the immunotherapy or of the immunogenic therapy the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18;
   determining a subject having breast cancer to be eligible, susceptible, qualified, suited or acceptable for treatment with the immunotherapy or with the immunogenic therapy, when the expression level quantified for the selected gene is within a pre-determined range of expression levels/expression level values/standard values of the gene wherein the pre-determined range of expression levels is indicative of/associated with positive outcome of the immunotherapy or of the immunogenic therapy; or
4) methods of determining prior to start, at start, or after start of an immunotherapy or of an immunogenic therapy in a subject having breast cancer the outcome of the immunotherapy or of the immunogenic therapy, comprising one or more steps of:
   quantifying in a sample obtained from the subject prior to start, at start, or early after start of the immunotherapy or of the immunogenic therapy the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18;
   determining (prior to start, at start, or after start of an immunotherapy or of an immunogenic therapy in a subject having breast cancer) the outcome of the immunotherapy or of the immunogenic therapy to be positive, when the expression level quantified for the selected gene is within a pre-determined range of expression levels/expression level values/standard values of the gene wherein the pre-determined range of expression levels is indicative of/associated with positive outcome of the immunotherapy or of the immunogenic therapy; or
5) methods of determining prior to start, at start, or after start of an immunotherapy or of an immunogenic therapy in a subject having breast cancer the response or to the immunotherapy or to the immunogenic therapy, comprising one or more steps of:
   quantifying in a sample obtained from the subject prior to start, at start, or early after start of the immunotherapy or of the immunogenic therapy the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18;
   determining (prior to start, at start, or after start of an immunotherapy or of an immunogenic therapy in a subject having breast cancer) the response to the immunotherapy or of the immunogenic therapy to be positive, when the expression level quantified for the selected gene is within a pre-determined range of expression levels/expression level values/standard values of the gene wherein the pre-determined range of expression levels is indicative of/associated with a positive response to the immunotherapy or to the immunogenic therapy; or
6) methods of determining prior to start, at start, or early after start of an immunotherapy or of an immunogenic therapy in a subject having breast cancer the early response to the immunotherapy or to the immunogenic therapy, comprising one or more steps of:
   quantifying in a sample obtained from the subject prior to start, at start, or early after start of the immunotherapy or of the immunogenic therapy the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18;
   determining (prior to start, at start, or after start of an immunotherapy or of an immunogenic therapy in a subject having breast cancer) the early response to the immunotherapy or of the immunogenic therapy to be positive, when the expression level quantified for the selected gene is within a pre-determined range of expression levels/expression level values/standard values of the gene wherein the pre-determined range of expression levels is indicative of/associated with a positive early response to the immunotherapy or to the immunogenic therapy; or
7) methods of determining prior to start, at start, or early after start of an immunotherapy or of an immunogenic therapy in a subject having breast cancer the T-cell expansion response to the immunotherapy or to the immunogenic therapy, comprising one or more steps of:
   quantifying in a sample obtained from the subject prior to start, at start, or early after start of the immunotherapy or of the immunogenic therapy the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18;
   determining (prior to start, at start, or after start of an immunotherapy or of an immunogenic therapy in a subject having breast cancer) the T-cell expansion response to the immunotherapy or of the immunogenic therapy to be positive, when the expression level quantified for the selected gene is within a pre-determined range of expression levels/expression level values/standard values of the gene wherein the pre-determined range of expression levels is indicative of/associated with a T-cell expansion response to the immunotherapy or to the immunogenic therapy; or
8) methods of determining prior to start, at start, or early after start of an immunotherapy or of an immunogenic therapy in a subject having breast cancer the early T-cell expansion response to the immunotherapy or to the immunogenic therapy, comprising one or more steps of:
   quantifying in a sample obtained from the subject prior to start, at start, or early after start of the immunotherapy or of the immunogenic therapy the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18;
   determining (prior to start, at start, or after start of an immunotherapy or of an immunogenic therapy in a subject having breast cancer) the T-cell expansion response to the immunotherapy or of the immunogenic therapy to be positive, when the expression level quantified for the selected gene is within a pre-determined range of expression levels/expression level values/standard values of the gene wherein the pre-determined range of expression levels is indicative of/associated with an early T-cell expansion response to the immunotherapy or to the immunogenic therapy; or
9) methods of monitoring the response of a subject having breast cancer to an immunotherapy or to an immunogenic therapy, comprising one or more steps of:
   quantifying in a sample obtained from the subject prior to start, at start, and/or (early) after start of the immunotherapy or of the immunogenic therapy the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18;
   determining the outcome of, the response to, the early response to, the T-cell response to, or the early T-cell response to the immunotherapy or of the immunogenic therapy to be positive, when the expression level quantified for the selected gene is within a pre-determined range of expression levels/expression level values/standard values of the gene wherein the pre-determined range of expression levels is indicative of/associated with a positive outcome of, positive response to, positive early response to the immunotherapy or to the immunogenic therapy; or is indicative of/associated with T-cell expansion or early T-cell expansion in response to the immunotherapy or to the immunogenic therapy.

In its bare essence, the invention relates to methods of breast tumor analysis or of breast tumor profiling, such methods comprising a step of quantifying in a sample obtained from the subject prior to start, at start, or early after start of the immunotherapy or of the immunogenic therapy the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18.

In one embodiment to these methods, the breast cancer is early breast cancer.

In a further embodiment, any expression level is determined in vitro in a sample obtained from the subject having breast cancer, or on a component isolated from that sample.

### Breast Cancer (BC)

About 20% of breast cancers (BCs) are human epidermal growth factor receptor 2 (HER2)-positive (HER2+) and usually are aggressive and fast-growing. Breast cancer can also be divided between hormone receptor-positive and hormone receptor-negative types, such as ER-positive (ER:estrogen receptor; ER-positive: responsive to estrogen) and/or PR-positive (PR: progesterone receptor; PR-positive: responsive to progesterone). About 80% of all BCs are ER-positive (ER+), and about 65% of the ER+ BCs are also PR-positive (PR+). ER+/PR+ BCs are more likely to respond to hormone therapy than ER−/PR− BCs. A fraction of about 10% to 20% BCs are HER2−, ER− and PR−, and are classified as triple negative (TN; TNBC). Early-stage breast cancer is breast cancer diagnosed at a stage wherein tumor(s) has(have) not yet spread beyond the breast or the axillary lymph nodes.

A subject having breast cancer is a subject having been diagnosed by any means of having breast cancer, i.e. of having one or more tumors being present in the breast tissue. A mammalian subject is in one embodiment a human subject or a human patient.

### Outcome of or Response to the Immunotherapy or the Immunogenic Therapy

Because anti-PD1 was delivered in a window-of-opportunity setting, direct exploration of whether T cell expansion translates into clinical benefit was not possible. However, in melanoma, peripheral T cell expansion occurring within three weeks after start of treatment correlates with improved clinical response to ICB six months later (Fairfax et al. 2020, Nat Med 26:193-199; Valpione et al. 2020, Nat Cancer 1:210-221). In view thereof, and because the number of cancer cells decreased on-treatment in BC patients displaying T-cell expansion, it is plausible and expected that T cell expansion in BC is likewise associated with clinical benefit of anti-PD1 therapy in BC patients. In addition, it was observed that the majority of expanding T cell clonotypes were already detected pre-treatment in the BC patients.

The response to the immunotherapy or to the immunogenic therapy in one embodiment is a clinical response such as duration of survival (e.g. overall survival), time to disease progression (TTP), response rates (e.g., complete response and partial response, stable disease, no response), length of progression-free survival, duration of response, quality of life, but can likewise be expressed in terms of having a therapeutic effect (such as treating/treatment cancer, inhibiting tumor progression or relapse, inhibiting tumor metastasis, and the like). The biomarkers as used in the current methods in particular are useful for dividing the subjects having breast cancer into likely responders to immunotherapy or immunogenic therapy on the one hand (positive outcome of, positive (early) response to, or (early) T cell expansion response to the immunotherapy or immunogenic therapy), and into likely non-responders to immunotherapy or immunogenic therapy on the other hand (no or negative outcome of, no or negative (early) response to, or no (early) T-cell expansion response to the immunotherapy or immunogenic therapy). The response of the subject having breast cancer to the immunotherapy or the immunogenic therapy herein is identified by means of checking expression levels of the one or more genes as mentioned hereinabove (and as will be explained in more detail hereinafter).

Sample/Prior to, at Start, after or Early after Start of Immunotherapy or Immunogenic Therapy

As indicated above, the methods can be performed on a sample, in particular a biological sample, obtained from the subject before or prior to starting the immunotherapy or immunogenic therapy. Alternatively, the methods can be performed on a (biological) sample obtained from the subject at the time point of starting/at the start of the immunotherapy or immunogenic therapy. In both cases, the therapeutic effects, if these would present in the subject, of the immunotherapy or immunogenic therapy have not yet taken off; in other words: the subject or the immune system of the subject has not yet reacted, adjusted or responded to the immunotherapy or immunogenic therapy.

Further alternatively, the methods can be performed on a (biological) sample obtained from the subject at the time point after start of the immunotherapy or immunogenic therapy. In one embodiment, “after start” is early after start such as to mimic as closely as possible the situation of prior to or at start. In another embodiment, performing the methods on a (biological) sample obtained from the subject after start time point provides further information about the absence of presence of a response of the subject to the immunotherapy or immunogenic therapy. In the latter case, the response of the subject to the immunotherapy or immunogenic therapy can be monitored and the methods are methods of monitoring the response of a subject having breast cancer to treatment with an immunotherapy or with an immunogenic therapy. More in particular, after start of the immunotherapy or immunogenic therapy is referring to a time point (at when the biological sample was obtained from the subject) after administration of a first cycle of administration of the immunotherapy or immunogenic therapy. More in particular, early after start of the immunotherapy or immunogenic therapy is referring to a time point between just and up to one week after the administration of the immunotherapy or immunogenic therapy, e.g. a time point between about 1 and 60 minutes, between about 1 minute and 2 hrs, between 1 minute and 6 hrs, between 1 minute and 12 hrs, between 1 minute and 24 hrs, between 1 to 24 hrs, between 1 to 2 days, between 1 to 3 days, between 1 to 4 days, between 1 to 5 days, between 1 to 6 days, between 1 to 7 days, between 1 to 9 days, between 1 to 15 days, between 1 to 20 days, between 7 to 15 days, at day 1, at day 2, at day 3, at day 4, at day 5, at day 6, at day 7, at day 8, at day 9, at day 10, at day 11, at day 12, at day 13, at day 14, at day 15, at day 16, at day 17, at day 18, at day 19 or at day 20 after the administration of the first cycle of the immunotherapy or immunogenic therapy. The “about 1 minute” (or around 1 minute) hereinabove refers to the closest feasible time point at which the biological sample can be obtained from the subject after administration of the first cycle of the immunotherapy or immunogenic therapy; i.e. it can be less than 1 minute.

Further alternatively, the methods can be performed at multiple time points such as a first time prior to start of the immunotherapy or immunogenic therapy; followed by at least at one second time point such as at start of the immunotherapy or immunogenic therapy and/or after starting the immunotherapy or immunogenic therapy.

The sample in particular is a biological sample, more in particular a biological sample comprising gene expression products derived from the breast tumor or breast cancer in the subject. Such biological sample can be a solid sample (e.g. solid biopsy, or part of an surgically excised or removed tumor) or a fluid sample (e.g. a liquid biopsy, such as non-invasive liquid biopsy or non-invasive sample). In particular the biological sample can be comprising cell-free genetic material shed from the tumor, and/or can be comprising circulating tumor cells. If multiple samples are obtained from the subject, then, in one embodiment, the first sample can be a solid biopsy sample whereas the at least one second sample can be a liquid biopsy sample; or vice versa. In another embodiment, the sample obtained from the subject after start/early after start of the immunotherapy or immunogenic therapy is a fluid sample.

A biological sample, or shortly sample, as referred to herein is any sample taken from/obtained from a mammal having a tumor that can serve as source of detection of expression of a biomarker of the invention. Such biological samples include tumor samples (such as obtained upon tumor biopsy), a bodily fluid sample or tumor exosomes from a mammal having a tumor.

Tumors are known to shed fragments of genomic DNA known as circulating tumor DNA or ctDNA (which is part of the circulating free DNA or cfDNA). For example, a bodily fluid sample can comprise, without limitation, bodily fluid, whole blood, serum, plasma, synovial fluid, lymphatic fluid, ascites fluid, interstitial or extracellular fluid, the fluid in spaces between cells, including gingival crevicular fluid, cerebrospinal fluid, saliva, mucous, sputum, phlegm, smegma, seminal fluid, ejaculate, sweat, tears, urine, fluid from nasal brushings, colonic washing fluid, fluid from a pap smear, vaginal fluid, vaginal flushing fluid, fluid from a hydrocele, pleural fluid, bronchoalveolar lavage fluid, discharge fluid from the nipple, aspiration fluid from a part of the body, colostrum, breast milk, ventricular fluid, any other bodily fluids. A bodily fluid can include saliva, blood, or serum. Bodily fluids of a subject can comprise ctDNA when the subject is having a tumor or cancer.

Tumors are known to produce exosomes (small membrane vesicles or microvesicles of endocytic origin). Compared to normal cells, the release of such exosomes by tumor cells is often elevated, which results in elevated levels of tumor-derived exosomes in the peripheral circulation and in bodily fluids such as serum or plasma, ascites, urine, and pleural effusions. This has led to the proposal to use such exosomes in diagnosis of cancer or for cancer biomarker analysis (e.g. Taylor & Gercel-Taylor 2008, Gynecol Oncol 110:13-21, and references cited therein). Even brain tumors such as glioblastoma produce exosomes that can be isolated from serum (Skog et al. 2008, Nature Cell Biol 10:1470-1476). Urine was reported to harbor exosomes of e.g. prostate cancer; ascites to harbor exosomes of e.g. colorectal cancer; and pleural effusions to harbor exosomes of e.g. mesothelioma, lung cancer, breast cancer, and ovarian cancer (van der Pol et al. 2012, Pharmacol Rev 64:676-705 and references cited therein). One way of enriching tumor exosomes prior to their analysis is ultracentrifugation of serum to form a pellet (e.g. Balaj et al. 2011, Nature Comm 2:180; Skog et al. 2008, Nature Cell Biol 10:1470-1476). When a specific target is present on the exosomes, then cell sorting technology can be used; epithelial tumors were for instance shown to produce exosomes containing epithelial cell adhesion molecule (EpCAM) and were purified from serum by magnetic activated cell sorting using anti-EpCAM coupled to magnetic beads (e.g. Taylor & Gercel-Taylor 2008, Gynecol Oncol 110:13-21).

### Biomarkers and Combinations Thereof.

Information on the individual biomarkers is provided hereinafter. While symbols for human genes are normally italicized (e.g. IGHG1) and symbols for protein are not italicized (e.g. IGHG1), this distinction is not or not consistently made hereinabove or hereinafter. The biomarker names are all-capital as the information is focusing on human genes. As part of the biomarker information, the chromosome allocation and the location of each biomarker gene on the allocated chromosome (start and end point; and forward (+) or reverse (−) strand where known) is included. Retrieving the actual nucleic acid sequence from the indicated allocation on the indicated chromosome is known to the skilled person, and the actual nucleic acid sequence can be retrieved e.g. by using a genome browser (e.g. https://genome.ucsc.edu/or https://www.ncbi.nlm.nih.gov/genome/) and by relying on the reference human genome used to delineate the biomarker genes of the invention.

### IGHG1

IGHG1 is also known as Immunoglobulin Heavy Constant Gamma 1 (G1m Marker or Gm Marker); Constant Region Of Heavy Chain Of IgG1; Immunoglobulin Heavy Constant Gamma 1; Ig Gamma-1 Chain C Region KOL or Region NIE or Region EU; or Ig Gamma-1 Chain C Region. At least 78 NCBI mRNA sequences exist, including those given in GenBank Accession nos AJ294730.1; AK057754.1; AK057775.1; AK093636.1; and AK097010.1. The human IGHG1 gene is located on chr14:105,736,343-105,743,071 (GRCh38/hg38; minus strand) or chr14:106,207,810-106,209,407 (GRCh37/hg19; minus strand).

### IGHG2

IGHG2 is also known as Immunoglobulin Heavy Constant Gamma 2 (G2m Marker or Gm Marker); Constant Region Of Heavy Chain Of IgG2; Immunoglobulin Heavy Constant Gamma 2; Immunoglobulin Gamma 2 (Gm Marker); Ig Gamma-2 Chain C Region DOT or Region TIL or Region ZIE; or Ig Gamma-2 Chain C Region. NCBI mRNA sequences are given in GenBank accession nos AJ294731.1; BC040042.1; BC062335.1; BX640623.1; and CR749861.1. The human IGHG2 gene is located on chr14:105,639,559-105,644,790 (GRCh38/hg38; minus strand) or chr14:106,109,540-106,111,126 (GRCh37/hg19; minus strand).

### IGHG3

IGHG3 is also known as Immunoglobulin Heavy Constant Gamma 3 (G3m Marker or Gm Marker); Constant Region Of Heavy Chain Of IgG3; Immunoglobulin Heavy Constant Gamma 3; Immunoglobulin Gamma 3 (Gm Marker); Secrete-Type Ig Gamma Heavy-Chain; Heavy Chain Disease Protein; Ig Gamma-3 Chain C Region; C Gamma; IgG3; or HDC. NCBI mRNA sequences include GenBank accession nos AK097307.1; AK097355.1; AK097572.1; AK097906.1; and AK098108.1. The human IGHG3 gene is located on chr14:105,764,503-105,771,405 (GRCh38/hg38; minus strand) or chr14:106,232,251-106,237,742 (GRCh37/hg19; minus strand).

### IGLC2

IGLC2 is also known as Immunoglobulin Lambda Constant 2; Immunoglobulin Lambda Constant 2 (Kern-Oz-Marker); Immunoglobulin Lambda Constant Region 2 (Kern-Oz-Marker); Ig Light-Chain, Partial Ke-Oz-Polypeptide, C-Term; Ig Lambda Chain C Region NIG-64 or Region Kern or Region SH or Region X; Ig Lambda-2 Chain C Region; C2 Segment; or IGLC. The human IGLC2 gene is located on chr22:22,900,976-22,901,437 (GRCh38/hg38; plus strand) or chr22:23,243,156-23,243,475(GRCh37/hg19; plus strand).

### IGKC

IGKC is also known as Immunoglobulin Kappa Constant; HCAK1; Immunoglobulin Kappa (Invariant Region); Immunoglobulin Kappa Light Chain (VJ); Immunoglobulin Kappa Constant Region; Ig Kappa Chain C Region CUM or Region ROY or Region AG or Region EU or Region OU or Region TI; Ig Kappa Chain C Region; IGKCD; or Km. NCBI mRNA sequences include GenBank accession nos AB245096.1; AF113887.1; AY894991.1; BC029444.1; BC034141.1. The human IGKC gene is located on chr2:88,857,161-88,857,683 (GRCh38/hg38; minus strand) or chr2:89,156,874-89,157,196 (GRCh37/hg19; minus strand).

### MZB1

MZB1 is also known as Marginal Zone B And B1 Cell Specific Protein; MEDA-7 or MEDA7; PACAP; PERp1; Plasma Cell-Induced Resident Endoplasmic Reticulum (ER) Protein; Mesenteric Oestrogen/Estrogen-Dependent Adipose Gene-7; Proapoptotic Caspase Adaptor Protein; Plasma Cell-Induced ER Protein 1; HSPC190; Caspase-2 Binding Protein; or MGC29506. The NCBI reference sequence mRNA is given in GenBank accession no NM_016459.4. The human MZB1 gene is located on chr5:139,387,467-139,390,081(GRCh38/hg38; minus strand) or chr5:138,723,156-138,725,602 (GRCh37/hg19; minus strand).

### HMGB2

HMGB2 is also known as High Mobility Group Box 2; High-Mobility Group (Nonhistone Chromosomal) Protein 2; High Mobility Group Protein B2; High Mobility Group Protein 2; or HMG2. NCBI reference sequence mRNAs are given in GenBank accession nos NM_001130688.1; NM_001130689.1; and NM_002129.4. The human HMGB gene is located on chr4:173,331,376-173,334,432 (GRCh38/hg38; minus strand) or chr4:174,252,527-174,255,509 (GRCh37/hg19; minus strand).

### SEC11C

SEC11C is also known as SEC11 Homolog C, Signal Peptidase Complex Subunit; SPCS4C; SPC21; Signal Peptidase Complex Catalytic Subunit SEC11C; Microsomal Signal Peptidase 21 KDa Subunit; Signal Peptidase Complex 21; SEC11-Like Protein 3; SPase 21 KDa Subunit; SEC11L3; SEC11 Homolog C (S. cerevisiae); SEC11-Like 3 (S. cerevisiae); SEC11 Homolog C; SEC11-Like 3; or EC 3.4.21.89. NCBI reference sequence mRNAs are given in GenBank accession nos NM_001307941.2 and NM_033280.4. The human SEC11C gene is located on chr18:59,139,866-59,158,837(GRCh38/hg38; plus strand) or chr18:56,807,116-56,826,064 (GRCh37/hg19; plus strand).

### BATF

BATF is also known as Basic Leucine Zipper ATF-Like Transcription Factor; B-ATF; SFA-2 or SFA2; Basic Leucine Zipper Transcriptional Factor ATF-Like; Basic Leucine Zipper Transcription Factor, ATF-Like; B-Cell-Activating Transcription Factor; Activating Transcription Factor B; SF-HT-Activated Gene 2 Protein; or BATF1. The NCBI reference sequence mRNA is given in GenBank accession no NM_006399.5. The human BATF gene is located on chr14:75,522,455-75,546,993 (GRCh38/hg38; plus strand) or chr14:75,988,812-76,013,335 (GRCh37/hg19; plus strand).

### CXCL13

CXCL13 is also known as C-X-C Motif Chemokine Ligand 13; BCA-1 or BCA1; BLC; Small Inducible Cytokine B Subfamily (Cys-X-Cys Motif), Member 13 (B-Cell Chemoattractant); B Cell-Attracting Chemokine 1; B Lymphocyte Chemoattractant; Small-Inducible Cytokine B13; C-X-C Motif Chemokine 13; B-Cell Chemoattractant; CXC Chemokine BLC; ANGIE, Angie or ANGIE2; SCYB13; BLR1L; B-Cell-Homing Chemokine (Ligand For Burkitt'S Lymphoma Receptor-1); Chemokine (C-X-C Motif) Ligand 13 (B-Cell Chemoattractant); or Chemokine (C-X-C Motif) Ligand 13. NCBI reference sequence mRNAs are given in GenBank accession nos NM_001371558.1 and NM_006419.3. The human CXCL13 gene is located on chr4:77,511,753-77,611,834 (GRCh38/hg38; plus strand) or chr4:78,432,907-78,532,988 (GRCh37/hg19; plus strand).

### CXCR6

CXCR6 is also known as C-X-C Motif Chemokine Receptor 6; STRL33; TYMSTR; BONZO; Chemokine (C-X-C Motif) Receptor 6; G-Protein Coupled Receptor STRL33 or Bonzo; C-X-C Chemokine Receptor Type 6; CDw186; CD186; CD186 Antigen; or CXC-R6. NCBI reference sequence mRNAs are given in GenBank accession nos NM_001386435.1; NM_001386436.1; NM_001386437.1 and NM_006564.2. The human CXCR6 gene is located on chr3:45,940,915-45,948,351(GRCh38/hg38; plus strand) or chr3:45,982,407-45,989,843 (GRCh37/hg19; plus strand).

### GZMB

GZMB is also known as Granzyme B; T-Cell Serine Protease 1-3E; Cathepsin G-Like 1; CTSGL1; CGL1; SECT; HLP; Granzyme B (Granzyme 2, Cytotoxic T-Lymphocyte-Associated Serine Esterase 1); Cytotoxic T-Lymphocyte Proteinase 2; Cytotoxic Serine Protease B; Human Lymphocyte Protein; Fragmentin 2; EC 3.4.21.79; CGL-1; CSP-B; CTLA1; CCPI; CSPB; C11; Cytotoxic T-Lymphocyte-Associated Serine Esterase 1; Lymphocyte Protease; Granzyme 2; or GRB. NCBI reference sequence mRNAs are given in GenBank accession nos NM_001346011.2 and NM_004131.6. The human GZMB gene is located on chr14:24,630,954-24,634,267(GRCh38/hg38; minus strand) or chr14:25,100,160-25,103,396 (GRCh37/hg19; minus strand).

### PDCD1

PDCD1 is also known as Programmed Cell Death 1; PD1 or PD-1; Systemic Lupus Erythematosus Susceptibility 2; Programmed Cell Death Protein 1; Protein PD-1; CD279 or CD279 antigen; HPD-1; HSLE1; Programmed Cell Death 1 Protein; SLEB2; or HPD-L. The NCBI reference sequence mRNA is given in GenBank accession nos NM_005018.3. The human PDCD1 gene is located on chr2:241,849,881-241,858,908 (GRCh38/hg38; minus strand) or chr2:242,792,036-242,801,046 (GRCh37/hg19; minus strand).

### TIGIT

TIGIT is also known as T Cell Immunoreceptor With Ig And ITIM Domains; V-Set And Immunoglobulin Domain-Containing Protein 9; V-Set And Transmembrane Domain-Containing Protein 3; T-Cell Immunoreceptor With Ig And ITIM Domains; VSIG9; VSTM3; VSIG9; V-Set And Immunoglobulin Domain Containing 9; Washington University Cell Adhesion Molecule; V-Set And Transmembrane Domain Containing 3; DKFZp667A205; FLJ39873; or WUCAM. The NCBI reference sequence mRNA is given in GenBank accession no NM_173799.4. The human TIGIT gene is located on chr3:114,276,913-114,329,747 (GRCh38/hg38; plus strand) or chr3:114,012,875-114,029,135 (GRCh37/hg19; plus strand).

### TNFRSF18

TNFRSF18 is also known as TNF Receptor Superfamily Member 18; AITR; GITR; Tumor Necrosis Factor Receptor Superfamily Member 18; Glucocorticoid-Induced TNFR-Related Protein; Activation-Inducible TNFR Family Receptor; CD357 or CD357 antigen; TNF Receptor Superfamily Activation-Inducible Protein; ENERGEN; or GITR-D. NCBI reference sequence mRNAs are given in GenBank accession nos NM_004195.3; NM_148901.2; and NM_148902.2. The human TNFRSF18 gene is located on chr1:1,203,508-1,207,901(GRCh38/hg38; minus strand) or chr1:1,138,888-1,141,972 (GRCh37/hg19; minus strand).

Any of the above-listed methods subject of the current invention may entail/encompass/comprise/include detecting, determining, measuring, assessing or quantifying the expression level of one or more of the listed genes.

In particular, any of the:
methods of selecting, identifying, choosing, or collecting (or, contrary, of refusing or rejecting) a subject or subjects having breast cancer for treatment with an immunotherapy or with an immunogenic therapy; or
methods of selecting, identifying, choosing, or collecting a subject or subjects having breast cancer fit or proper for treatment with an immunotherapy or with an immunogenic therapy; or
methods of determining eligibility, susceptibility, qualification, suitability or acceptability of a subject having breast cancer for treatment with an immunotherapy or with an immunogenic therapy; or
methods of determining prior to start, at start, or after start of an immunotherapy or of an immunogenic therapy in a subject having breast cancer the outcome of the immunotherapy or of the immunogenic therapy; or
methods of determining prior to start, at start, or after start of an immunotherapy or of an immunogenic therapy in a subject having breast cancer the response or to the immunotherapy or to the immunogenic therapy; or
methods of determining prior to start, at start, or early after start of an immunotherapy or of an immunogenic therapy in a subject having breast cancer the early response to the immunotherapy or to the immunogenic therapy; or
methods of determining prior to start, at start, or early after start of an immunotherapy or of an immunogenic therapy in a subject having breast cancer the T-cell expansion response to the immunotherapy or to the immunogenic therapy; or
methods of determining prior to start, at start, or early after start of an immunotherapy or of an immunogenic therapy in a subject having breast cancer the early T-cell expansion response to the immunotherapy or to the immunogenic therapy; or
methods of monitoring the response of a subject having breast cancer to an immunotherapy or to an immunogenic therapy;
   may entail/encompass/comprise/include detecting, determining, measuring, assessing or quantifying the expression level of one or more genes as included in the following embodiments:
1) in one embodiment, the gene IGHG1;
2) in one embodiment, the gene IGHG2;
3) in one embodiment, the gene IGHG3;
4) in one embodiment, the gene IGLC2;
5) in one embodiment, the gene IGKC;
6) in one embodiment, the gene HMGB2;
7) in one embodiment, the gene MZB1;
8) in one embodiment, the gene SEC11C;
9) in one embodiment, the gene BATF;
10) in one embodiment, the gene CXCL13;
11) in one embodiment, the gene CXCR6;
12) in one embodiment, the gene GZMB;
13) in one embodiment, the gene PDCD1;
14) in one embodiment, the gene TIGIT;
15) in one embodiment, the gene TNFRSF18;
16) in one embodiment, 1, 2 or more (2, 3, or 4) genes selected from the group (consisting of) IGHG1, IGHG2, IGHG3, and IGLC2; in a further optional embodiment combined with a further gene selected from group (consisting of) the genes of embodiments 5) to 15);
17) in one embodiment, 1, 2 or more (2, 3, 4, or 5) genes selected from the group (consisting of) IGHG1, IGHG2, IGHG3, IGLC2, and IGKC; in a further optional embodiment combined with a (further) gene selected from group (consisting of) the genes of embodiments 6) to 15);
18) in one embodiment, 1, 2 or more (2, 3, 4, or 5) genes selected from the group (consisting of) IGHG1, IGHG2, IGHG3, IGLC2, and HMGB2; in a further optional embodiment combined with a (further) gene selected from group (consisting of) the genes of embodiments 5) and 7) to 15);
19) in one embodiment, 1, 2 or more (2, 3, 4, 5, or 6) genes selected from the group (consisting of) IGHG1, IGHG2, IGHG3, IGLC2, IGKC, and HMGB2; in a further optional embodiment combined with a (further) gene selected from group (consisting of) the genes of embodiments 7) to 15);
20) in one embodiment, 1, 2 or more genes (2, 3, or 4) selected from the group (consisting of) MZB1, SEC11C, BATF, and CXCR6;
21) in one embodiment, 1, 2 or more genes (2, 3, 4, 5, 6, or 7) selected from the group (consisting of) MZB1, SEC11C, BATF, CXCR6, PDCD1, TIGIT, and TNFRSF18;
22) in one embodiment, 1, 2 or more genes (2, 3, 4, or 5) selected from the group (consisting of) MZB1, SEC11C, BATF, CXCR6, and HMGB2;
23) in one embodiment, 1, 2 or more genes (2, 3, 4, 5, 6, 7, or 8) selected from the group (consisting of) MZB1, SEC11C, BATF, CXCR6, PDCD1, TIGIT, TNFRSF18, and HMGB2;
24) in one embodiment, 1, 2 or more genes (2, 3, 4, 5, 6, 7, 8 or 9) selected from the group (consisting of) MZB1, SEC11C, BATF, CXCL13, CXCR6, GZMB, PDCD1, TIGIT, and TNFRSF18;
25) in one embodiment, 1, 2 or more genes (2, 3, 4, 5, 6, 7, 8, 9 or 10) selected from the group (consisting of) MZB1, SEC11C, BATF, CXCL13, CXCR6, GZMB, PDCD1, TIGIT, TNFRSF18, and HMGB2;
26) in one embodiment, 1, 2 or more genes (2, 3, 4, 5, 6, 7, or 8) selected from the group (consisting of) IGHG1, IGHG2, IGHG3, IGLC2, MZB1, SEC11C, BATF, and CXCR6;
27) in one embodiment, 1, 2 or more genes (2, 3, 4, 5, 6, 7, 8, or 9) selected from the group (consisting of) IGHG1, IGHG2, IGHG3, IGLC2, IGKC, MZB1, SEC11C, BATF, and CXCR6;
28) in one embodiment, 1, 2 or more genes (2, 3, 4, 5, 6, 7, 8, or 9) selected from the group (consisting of) IGHG1, IGHG2, IGHG3, IGLC2, HMGB2, MZB1, SEC11C, BATF, and CXCR6;
29) in one embodiment, 1, 2 or more genes (2, 3, 4, 5, 6, 7, 8, 9, or 10) selected from the group (consisting of) IGHG1, IGHG2, IGHG3, IGLC2, IGKC, HMGB2, MZB1, SEC11C, BATF, and CXCR6;
30) in one embodiment, 1, 2 or more genes (2, 3, 4, 5, 6, 7, 8, 9, 10, or 11) selected from the group (consisting of) IGHG1, IGHG2, IGHG3, IGLC2, MZB1, SEC11C, BATF, CXCR6, PDCD1, TIGIT, and TNFRSF18;
31) in one embodiment, 1, 2 or more genes (2, 3, 4, 5, 6, 7, 8, 9, 10, 11, or 12) selected from the group (consisting of) IGHG1, IGHG2, IGHG3, IGLC2, IGKC, MZB1, SEC11C, BATF, CXCR6, PDCD1, TIGIT, and TNFRSF18
32) in one embodiment, 1, 2 or more genes (2, 3, 4, 5, 6, 7, 8, 9, 10, 11, or 12) selected from the group (consisting of) IGHG1, IGHG2, IGHG3, IGLC2, HMGB2, MZB1, SEC11C, BATF, CXCR6, PDCD1, TIGIT, and TNFRSF18;
33) in one embodiment, 1, 2 or more genes (2, 3, 4, 5, 6, 7, 8, 9, 10, 11, 12, or 13) selected from the group (consisting of) IGHG1, IGHG2, IGHG3, IGLC2, IGKC, HMGB2, MZB1, SEC11C, BATF, CXCR6, PDCD1, TIGIT, and TNFRSF18;
34) in one embodiment, 1, 2 or more genes (2, 3, 4, 5, 6, 7, 8, 9, 10, 11, 12, or 13) selected from the group (consisting of) IGHG1, IGHG2, IGHG3, IGLC2, MZB1, SEC11C, BATF, CXCL13, CXCR6, GZMB, PDCD1, TIGIT, and TNFRSF18;
35) in one embodiment, 1, 2 or more genes (2, 3, 4, 5, 6, 7, 8, 9, 10, 11, 12, 13, or 14) selected from the group (consisting of) IGHG1, IGHG2, IGHG3, IGLC2, IGKC, MZB1, SEC11C, BATF, CXCL13, CXCR6, GZMB, PDCD1, TIGIT, and TNFRSF18;
36) in one embodiment, 1, 2 or more genes (2, 3, 4, 5, 6, 7, 8, 9, 10, 11, 12, 13, or 14) selected from the group (consisting of) IGHG1, IGHG2, IGHG3, IGLC2, HMGB2, MZB1, SEC11C, BATF, CXCL13, CXCR6, GZMB, PDCD1, TIGIT, and TNFRSF18;
37) in one embodiment, 1, 2 or more genes (2, 3, 4, 5, 6, 7, 8, 9, 10, 11, 12, 13, 14, or 15) selected from the group (consisting of) IGHG1, IGHG2, IGHG3, IGLC2, IGKC, HMGB2, MZB1, SEC11C, BATF, CXCL13, CXCR6, GZMB, PDCD1, TIGIT, and TNFRSF18;
38) in clarification of embodiment 16) hereinabove, the combinations of the 2 or more genes include, without intent of being complete: IGHG1 and IGHG2; IGHG1 and IGHG3; IGHG1 and IGLC2; IGHG2 and IGHG3; IGHG2 and IGLC2; IGHG3 and IGLC2; IGHG1, IGHG2, and IGHG3; IGHG1, IGHG2, and IGLC2; IGHG1, IGHG3, and IGLC2; IGHG2, IGHG3, and IGLC2; or IGHG1, IGHG2, IGHG3 and IGLC2;
39) in clarification of the embodiment 17) hereinabove, the combinations of the 2 or more genes include any of the individual gene combinations listed in 38) hereinabove to which the gene IGKC is added, and further include the gene combinations IGHG1 and IGKC; IGHG2 and IGKC; IGHG3 and IGKC; and IGLC2 and IGKC;
40) in clarification of the embodiment 18) hereinabove, the combinations of the 2 or more genes include any of the individual gene combinations listed in 38) hereinabove to which the gene HMGB2 is added, and further include the gene combinations IGHG1 and HMGB2; IGHG2 and HMGB2; IGHG3 and HMGB2; and IGLC2 and HMGB2;
41) in clarification of the embodiment 19) hereinabove, the combinations of the 2 or more genes include any of the individual gene combinations listed in 38) hereinabove to which the gene IGKC is added, to which the gene HMGB2 is added, or to which the genes IGKC and HMGB2 are added; and further include the gene combinations IGHG1 and IGKC; IGHG2 and IGKC; IGHG3 and IGKC; IGLC2 and IGKC; IGHG1 and HMGB2; IGHG2 and HMGB2; IGHG3 and HMGB2; and IGLC2 and HMGB2;
42) in clarification of the embodiment 20) hereinabove, the combinations of the 2 or more genes include, without intent of being complete: MZB1 and SEC11C; MZB1 and BATF; MZB1 and CXCR6; SEC11C and BATF; SEC11C and CXCR6; BATF and CXCR6; MZB1, SEC11C, and BATF; MZB1, SEC11C, and CXCR6; MZB1, BATF, and CXCR6; SEC11C, BATF, and CXCR6; and MZB1, SEC11C, BATF, and CXCR6;
43) in clarification of the embodiment 21) hereinabove, the combinations of the 2 or more genes include, without intent of being complete: MZB1 and SEC11C; MZB1 and BATF; MZB1 and CXCR6; MZB1 and PDCD1; MZB1 and TIGIT; MZB1 and TNFRSF18; SEC11C and BATF; SEC11C and CXCR6; SEC11C and PDCD1; SEC11C and TIGIT; SEC11C and TNFRSF18; BATF and CXCR6; BATF and PDCD1; BATF and TIGIT; BATF and TNFRSF18; CXCR6 and PDCD1; CXCR6 and TIGIT; CXCR6 and TNFRSF18; PDCD1 and TIGIT; PDCD1 and TNFRSF18; TIGIT and TNFRSF18; MZB1, SEC11C, and BATF; MZB1, SEC11C, and CXCR6; MZB1, SEC11C, and PDCD1; MZB1, SEC11C, and TIGIT; MZB1, SEC11C, and TNFRSF18; MZB1, BATF, and CXCR6; MZB1, BATF, and PDCD1; MZB1, BATF, and TIGIT; MZB1, BATF, and TNFRSF18; MZB1, CXCR6, and PDCD1; MZB1, CXCR6, and TIGIT; MZB1, CXCR6, and TNFRSF18; MZB1, PDCD1, and TIGIT; MZB1, PDCD1, and TNFRSF18; MZB1, TIGIT, and TNFRSF18; SEC11C, BATF, and CXCR6; SEC11C, BATF, and PDCD1; SEC11C, BATF, and TIGIT; SEC11C, BATF, and TNFRSF18; SEC11C, CXCR6, and PDCD1; SEC11C, CXCR6, and TIGIT; SEC11C, CXCR6, and TNFRSF18; SEC11C, PDCD1, and TIGIT; SEC11C, PDCD1, and TNFRSF18; SEC11C, TIGIT, and TNFRSF18; BATF, CXCR6, and PDCD1; BATF, CXCR6, and TIGIT; BATF, CXCR6, and TNFRSF18; BATF, PDCD1, and TIGIT; BATF, PDCD1, and TNFRSF18; BATF, TIGIT, and TNFRSF18; CXCR6, PDCD1, and TIGIT; CXCR6, PDCD1, and TNFRSF18; PDCD1, TIGIT, and TNFRSF18; MZB1, SEC11C, BATF, and CXCR6; MZB1, SEC11C, BATF, and PDCD1; MZB1, SEC11C, BATF, and TIGIT; MZB1, SEC11C, BATF, and TNFRSF18; MZB1, SEC11C, CXCR6, and PDCD1; MZB1, SEC11C, CXCR6, and TIGIT; MZB1, SEC11C, CXCR6, and TNFRSF18; MZB1, SEC11C, PDCD1, and TIGIT; MZB1, SEC11C, PDCD1, and TNFRSF18; MZB1, SEC11C, TIGIT, and TNFRSF18; MZB1, BATF, CXCR6, and PDCD1; MZB1, BATF, CXCR6, and TIGIT; MZB1, BATF, CXCR6, and TNFRSF18; MZB1, BATF, PDCD1, and TIGIT; MZB1, BATF, PDCD1, and TNFRSF18; MZB1, BATF, TIGIT, and TNFRSF18; MZB1, CXCR6, PDCD1, and TIGIT; MZB1, CXCR6, PDCD1, and TNFRSF18; MZB1, CXCR6, TIGIT, and TNFRSF18; MZB1, PDCD1, TIGIT, and TNFRSF18; SEC11C, BATF, CXCR6, and PDCD1; SEC11C, BATF, CXCR6, and TIGIT; SEC11C, BATF, CXCR6, and TNFRSF18; SEC11C, BATF, PDCD1, and TIGIT; SEC11C, BATF, PDCD1, and TNFRSF18; SEC11C, BATF, TIGIT, and TNFRSF18; SEC11C, CXCR6, PDCD1, and TIGIT; SEC11C, CXCR6, PDCD1, and TNFRSF18; SEC11C, CXCR6, TIGIT, and TNFRSF18; SEC11C, PDCD1, TIGIT, and TNFRSF18; BATF, CXCR6, PDCD1, and TIGIT; BATF, CXCR6, PDCD1, and TNFRSF18; BATF, CXCR6, TIGIT, and TNFRSF18; CXCR6, PDCD1, TIGIT, and TNFRSF18; MZB1, SEC11C, BATF, CXCR6, and PDCD1; MZB1, SEC11C, BATF, CXCR6, and TIGIT; MZB1, SEC11C, BATF, CXCR6, and TNFRSF18; MZB1, SEC11C, CXCR6, PDCD1, and TIGIT; MZB1, SEC11C, CXCR6, PDCD1, and TNFRSF18; MZB1, SEC11C, PDCD1, TIGIT, and TNFRSF18; MZB1, BATF, CXCR6, PDCD1, and TIGIT; MZB1, BATF, CXCR6, PDCD1, and TNFRSF18; MZB1, BATF, CXCR6, TIGIT, and TNFRSF18; MZB1, BATF, PDCD1, TIGIT, and TNFRSF18; MZB1, CXCR6, PDCD1, TIGIT, and TNFRSF18; SEC11C, BATF, CXCR6, PDCD1, and TIGIT; SEC11C, BATF, CXCR6, PDCD1, and TNFRSF18; SEC11C, BATF, PDCD1, TIGIT, and TNFRSF18; BATF, CXCR6, PDCD1, TIGIT, and TNFRSF18; MZB1, SEC11C, BATF, CXCR6, PDCD1, and TIGIT; MZB1, SEC11C, BATF, CXCR6, PDCD1, and TNFRSF18; MZB1, BATF, CXCR6, PDCD1, TIGIT, and TNFRSF18; MZB1, SEC11C, CXCR6, PDCD1, TIGIT, and TNFRSF18; MZB1, SEC11C, BATF, PDCD1, TIGIT, and TNFRSF18; MZB1, SEC11C, BATF, CXCR6, TIGIT, and TNFRSF18; SEC11C, BATF, CXCR6, PDCD1, TIGIT, and TNFRSF18; and MZB1, SEC11C, BATF, CXCR6, PDCD1, TIGIT, and TNFRSF18;
44) in clarification of the embodiment 22) hereinabove, the combinations of the 2 or more genes include any of the individual gene combinations listed in 42) hereinabove to which the gene HMGB2 is added, and further include the gene combinations MZB1 and HMGB2; SEC11C and HMGB2; BATF and HMGB2; and CXCR6 and HMGB2;
45) in clarification of the embodiment 23) hereinabove, the combinations of the 2 or more genes include any of the individual gene combinations listed in 43) hereinabove to which the gene HMGB2 is added, and further include the gene combinations MZB1 and HMGB2; SEC11C and HMGB2; BATF and HMGB2; and CXCR6 and HMGB2; PDCD1 and HMGB2; TIGIT and HMGB2; and TNFRSF18 and HMGB2;
46) in clarification of embodiment 24) hereinabove:
   the combinations of 2 genes include, without intent of being complete: MZB1 and SEC11C; MZB1 and BATF; MZB1 and CXCL13; MZB1 and CXCR6; MZB1 and GZMB; MZB1 and PDCD1; MZB1 and TIGIT; MZB1 and TNFRSF18; SEC11C and BATF; SEC11C and CXCL13; SEC11C and CXCR6; SEC11C and GZMB; SEC11C and PDCD1; SEC11C and TIGIT; SEC11C and TNFRSF18; BATF and CXCL13; BATF and CXCR6; BATF and GZMB; BATF and PDCD1; BATF and TIGIT; BATF and TNFRSF18; CXCL13 and CXCR6; CXCL13 and GZMB; CXCL13 and PDCD1; CXCL13 and TIGIT; CXCL13 and TNFRSF18; CXCR6 and GZMB; CXCR6 and PDCD1; CXCR6 and TIGIT; CXCR6 and TNFRSF18; GZMB and PDCD1; GZMB and TIGIT; GZMB and TNFRSF18; PDCD1 and TIGIT; PDCD1 and TNFRSF18; and TIGIT and TNFRSF18;
   the combination of 3 genes include, without intent of being complete: MZB1, SEC11C, and BATF; MZB1, SEC11C, and CXCL13; MZB1, SEC11C, and CXCR6; MZB1, SEC11C, and GZMB; MZB1, SEC11C, and PDCD1; MZB1, SEC11C, and TIGIT; MZB1, SEC11C, and TNFRSF18; MZB1, BATF, and CXCL13; MZB1, BATF, and CXCR6; MZB1, BATF, and GZMB; MZB1, BATF, and PDCD1; MZB1, BATF, and TIGIT; MZB1, BATF, and TNFRSF18; MZB1, CXCL13, and CXCR6; MZB1, CXCL13, and GZMB; MZB1, CXCL13, and PDCD1; MZB1, CXCL13, and TIGIT; MZB1, CXCL13, and TNFRSF18; MZB1, CXCR6, and GZMB; MZB1, CXCR6, and PDCD1; MZB1, CXCR6, and TIGIT; MZB1, CXCR6, and TNFRSF18; MZB1, GZMB, and PDCD1; MZB1, GZMB, and TIGIT; MZB1, GZMB, and TNFRSF18; MZB1, PDCD1, and TIGIT; MZB1, PDCD1, and TNFRSF18; MZB1, TIGIT, and TNFRSF18; SEC11C, BATF, and CXCL13; SEC11C, BATF, and CXCR6; SEC11C, BATF, and GZMB; SEC11C, BATF, and PDCD1; SEC11C, BATF, and TIGIT; SEC11C, BATF, and TNFRSF18; SEC11C, CXCL13, and CXCR6; SEC11C, CXCL13, and GZMB; SEC11C, CXCL13, and PDCD1; SEC11C, CXCL13, and TIGIT; SEC11C, CXCL13, and TNFRSF18; SEC11C, CXCR6, and GZMB; SEC11C, CXCR6, and PDCD1; SEC11C, CXCR6, and TIGIT; SEC11C, CXCR6, and TNFRSF18; SEC11C, GZMB, and PDCD1; SEC11C, GZMB, and TIGIT; SEC11C, GZMB, and TNFRSF18; SEC11C, PDCD1, and TIGIT; SEC11C, PDCD1, and TNFRSF18; SEC11C, TIGIT, and TNFRSF18; BATF, CXCL13, and CXCR6; BATF, CXCL13, and GZMB; BATF, CXCL13, and PDCD1; BATF, CXCL13, and TIGIT; BATF, CXCL13, and TNFRSF18; BATF, CXCR6, and GZMB; BATF, CXCR6, and PDCD1; BATF, CXCR6, and TIGIT; BATF, CXCR6, and TNFRSF18; BATF, GZMB, and PDCD1; BATF, GZMB, and TIGIT; BATF, GZMB, and TNFRSF18; BATF, PDCD1, and TIGIT; BATF, PDCD1, and TNFRSF18; BATF, TIGIT, and TNFRSF18; CXCL13, CXCR6, and GZMB; CXCL13, CXCR6, and PDCD1; CXCL13, CXCR6, and TIGIT; CXCL13, CXCR6, and TNFRSF18; CXCL13, GZMB, and PDCD1; CXCL13, GZMB, and TIGIT; CXCL13, GZMB, and TNFRSF18; CXCL13, PDCD1, and TIGIT; CXCL13, PDCD1, and TNFRSF18; CXCL13, TIGIT, and TNFRSF18; CXCR6, GZMB, and PDCD1; CXCR6, GZMB, and TIGIT; CXCR6, GZMB, and TNFRSF18; CXCR6, PDCD1, and TIGIT; CXCR6, PDCD1, and TNFRSF18; CXCR6, TIGIT, and TNFRSF18; GZMB, PDCD1, and TIGIT; GZMB, PDCD1, and TNFRSF18; and PDCD1, TIGIT, and TNFRSF18;
   the combination of 4 genes include, without intent of being complete: MZB1, SEC11C, BATF, and CXCL13; MZB1, SEC11C, BATF, and CXCR6; MZB1, SEC11C, BATF, and GZMB; MZB1, SEC11C, BATF, and PDCD1; MZB1, SEC11C, BATF, and TIGIT; MZB1, SEC11C, BATF, and TNFRSF18; MZB1, SEC11C, CXCL13, and CXCR6; MZB1, SEC11C, CXCL13, and GZMB; MZB1, SEC11C, CXCL13, and PDCD1; MZB1, SEC11C, CXCL13, and TIGIT; MZB1, SEC11C, CXCL13, and TNFRSF18; MZB1, SEC11C, CXCR6, and GZMB; MZB1, SEC11C, CXCR6, and PDCD1; MZB1, SEC11C, CXCR6, and TIGIT; MZB1, SEC11C, CXCR6, and TNFRSF18; MZB1, SEC11C, GZMB, and PDCD1; MZB1, SEC11C, GZMB, and TIGIT; MZB1, SEC11C, GZMB, and TNFRSF18; MZB1, SEC11C, PDCD1, and TIGIT; MZB1, SEC11C, PDCD1, and TNFRSF18; MZB1, SEC11C, TIGIT, and TNFRSF18; MZB1, BATF, CXCL13, and CXCR6; MZB1, BATF, CXCL13, and GZMB; MZB1, BATF, CXCL13, and PDCD1; MZB1, BATF, CXCL13, and TIGIT; MZB1, BATF, CXCL13, and TNFRSF18; MZB1, BATF, CXCR6, and GZMB; MZB1, BATF, CXCR6, and PDCD1; MZB1, BATF, CXCR6, and TIGIT; MZB1, BATF, CXCR6, and TNFRSF18; MZB1, BATF, GZMB, and PDCD1; MZB1, BATF, GZMB, and TIGIT; MZB1, BATF, GZMB, and TNFRSF18; MZB1, BATF, PDCD1, and TIGIT; MZB1, BATF, PDCD1, and TNFRSF18; MZB1, BATF, TIGIT, and TNFRSF18; MZB1, CXCL13, CXCR6, and GZMB; MZB1, CXCL13, CXCR6, and PDCD1; MZB1, CXCL13, CXCR6, and TIGIT; MZB1, CXCL13, CXCR6, and TNFRSF18; MZB1, CXCL13, GZMB, and PDCD1; MZB1, CXCL13, GZMB, and TIGIT; MZB1, CXCL13, GZMB, and TNFRSF18; MZB1, CXCL13, PDCD1, and TIGIT; MZB1, CXCL13, PDCD1, and TNFRSF18; MZB1, CXCL13, TIGIT, and TNFRSF18; MZB1, CXCR6, GZMB, and PDCD1; MZB1, CXCR6, GZMB, and TIGIT; MZB1, CXCR6, GZMB, and TNFRSF18; MZB1, CXCR6, PDCD1, and TIGIT; MZB1, CXCR6, PDCD1, and TNFRSF18; MZB1, CXCR6, TIGIT, and TNFRSF18; MZB1, GZMB, PDCD1, and TIGIT; MZB1, GZMB, PDCD1, and TNFRSF18; MZB1, PDCD1, TIGIT, and TNFRSF18; MZB1, GZMB, TIGIT, and TNFRSF18; SEC11C, BATF, CXCL13, and CXCR6; SEC11C, BATF, CXCL13, and GZMB; SEC11C, BATF, CXCL13, and PDCD1; SEC11C, BATF, CXCL13, and TIGIT; SEC11C, BATF, CXCL13, and TNFRSF18; SEC11C, BATF, CXCR6, and GZMB; SEC11C, BATF, CXCR6, and PDCD1; SEC11C, BATF, CXCR6, and TIGIT; SEC11C, BATF, CXCR6, and TNFRSF18; SEC11C, BATF, GZMB, and PDCD1; SEC11C, BATF, GZMB, and TIGIT; SEC11C, BATF, GZMB, and TNFRSF18; SEC11C, BATF, PDCD1, and TIGIT; SEC11C, BATF, PDCD1, and TNFRSF18; SEC11C, BATF, TIGIT, and TNFRSF18; SEC11C, CXCL13, CXCR6, and GZMB; SEC11C, CXCL13, CXCR6, and PDCD1; SEC11C, CXCL13, CXCR6, and TIGIT; SEC11C, CXCL13, CXCR6, and TNFRSF18; SEC11C, CXCL13, GZMB, and PDCD1; SEC11C, CXCL13, GZMB, and TIGIT; SEC11C, CXCL13, GZMB, and TNFRSF18; SEC11C, CXCL13, PDCD1, and TIGIT; SEC11C, CXCL13, PDCD1, and TNFRSF18; SEC11C, CXCL13, TIGIT, and TNFRSF18; SEC11C, CXCR6, GZMB, and PDCD1; SEC11C, CXCR6, GZMB, and TIGIT; SEC11C, CXCR6, GZMB, and TNFRSF18; SEC11C, CXCR6, PDCD1, and TIGIT; SEC11C, CXCR6, PDCD1, and TNFRSF18; SEC11C, CXCR6, TIGIT, and TNFRSF18; SEC11C, GZMB, PDCD1, and TIGIT; SEC11C, GZMB, PDCD1, and TNFRSF18; SEC11C, PDCD1, TIGIT, and TNFRSF18; BATF, CXCL13, CXCR6, and GZMB; BATF, CXCL13, CXCR6, and PDCD1; BATF, CXCL13, CXCR6, and TIGIT; BATF, CXCL13, CXCR6, and TNFRSF18; BATF, CXCL13, GZMB, and PDCD1; BATF, CXCL13, GZMB, and TIGIT; BATF, CXCL13, GZMB, and TNFRSF18; BATF, CXCL13, PDCD1, and TIGIT; BATF, CXCL13, PDCD1, and TNFRSF18; BATF, CXCL13, TIGIT, and TNFRSF18; BATF, CXCR6, GZMB, and PDCD1; BATF, CXCR6, GZMB, and TIGIT; BATF, CXCR6, GZMB, and TNFRSF18; BATF, CXCR6, PDCD1, and TIGIT; BATF, CXCR6, PDCD1, and TNFRSF18; BATF, CXCR6, PDCD1, TIGIT, and TNFRSF18; BATF, GZMB, PDCD1, and TIGIT; BATF, GZMB, PDCD1, and TNFRSF18; BATF, GZMB, TIGIT, and TNFRSF18; BATF, PDCD1, TIGIT, and TNFRSF18; CXCL13, CXCR6, GZMB, and PDCD1; CXCL13, CXCR6, GZMB, and TIGIT; CXCL13, CXCR6, GZMB, and TNFRSF18; CXCL13, CXCR6, PDCD1, and TIGIT; CXCL13, CXCR6, PDCD1, and TNFRSF18; CXCL13, CXCR6, TIGIT, and TNFRSF18; CXCL13, GZMB, PDCD1, and TIGIT; CXCL13, GZMB, PDCD1, and TNFRSF18; CXCL13, PDCD1, TIGIT, and TNFRSF18; CXCL13, GZMB, TIGIT, and TNFRSF18; CXCL13, PDCD1, TIGIT, and TNFRSF18; CXCR6, GZMB, PDCD1, and TIGIT; CXCR6, GZMB, PDCD1, and TNFRSF18; CXCR6, GZMB, TIGIT, and TNFRSF18; CXCR6, PDCD1, TIGIT, and TNFRSF18; and GZMB, PDCD1, TIGIT, and TNFRSF18;
   the combination of 5 genes include, without intent of being complete: MZB1, SEC11C, BATF, CXCL13, and CXCR6; MZB1, SEC11C, BATF, CXCL13, and GZMB; MZB1, SEC11C, BATF, CXCL13, and PDCD1; MZB1, SEC11C, BATF, CXCL13, and TIGIT; MZB1, SEC11C, BATF, CXCL13, and TNFRSF18; MZB1, SEC11C, BATF, CXCR6, and GZMB; MZB1, SEC11C, BATF, CXCR6, and PDCD1; MZB1, SEC11C, BATF, CXCR6, and TIGIT; MZB1, SEC11C, BATF, CXCR6, and TNFRSF18; MZB1, SEC11C, BATF, GZMB, and PDCD1; MZB1, SEC11C, BATF, GZMB, and TIGIT; MZB1, SEC11C, BATF, GZMB, and TNFRSF18; MZB1, SEC11C, BATF, PDCD1, and TIGIT; MZB1, SEC11C, BATF, PDCD1, and TNFRSF18; MZB1, SEC11C, BATF, TIGIT, and TNFRSF18; MZB1, SEC11C, CXCL13, CXCR6, and GZMB; MZB1, SEC11C, CXCL13, CXCR6, and PDCD1; MZB1, SEC11C, CXCL13, CXCR6, and TIGIT; MZB1, SEC11C, CXCL13, CXCR6, and TNFRSF18; MZB1, SEC11C, CXCL13, GZMB, and PDCD1; MZB1, SEC11C, CXCL13, GZMB, and TIGIT; MZB1, SEC11C, CXCL13, GZMB, and TNFRSF18; MZB1, SEC11C, CXCL13, PDCD1, and TIGIT; MZB1, SEC11C, CXCL13, PDCD1, and TNFRSF18; MZB1, SEC11C, CXCL13, TIGIT, and TNFRSF18; MZB1, SEC11C, CXCR6, GZMB, and PDCD1; MZB1, SEC11C, CXCR6, GZMB, and TIGIT; MZB1, SEC11C, CXCR6, GZMB, and TNFRSF18; MZB1, SEC11C, CXCR6, PDCD1, and TIGIT; MZB1, SEC11C, CXCR6, PDCD1, and TNFRSF18; MZB1, SEC11C, CXCR6, TIGIT, and TNFRSF18; MZB1, SEC11C, GZMB, PDCD1, and TIGIT; MZB1, SEC11C, GZMB, PDCD1, and TNFRSF18; MZB1, SEC11C, GZMB, TIGIT, and TNFRSF18; MZB1, SEC11C, PDCD1, TIGIT, and TNFRSF18; MZB1, BATF, CXCL13, CXCR6, and GZMB; MZB1, BATF, CXCL13, CXCR6, and PDCD1; MZB1, BATF, CXCL13, CXCR6, and TIGIT; MZB1, BATF, CXCL13, CXCR6, and TNFRSF18; MZB1, BATF, CXCL13, GZMB, and PDCD1; MZB1, BATF, CXCL13, GZMB, and TIGIT; MZB1, BATF, CXCL13, GZMB, and TNFRSF18; MZB1, BATF, CXCL13, PDCD1, and TIGIT; MZB1, BATF, CXCL13, PDCD1, and TNFRSF18; MZB1, BATF, CXCL13, TIGIT, and TNFRSF18; MZB1, BATF, CXCR6, GZMB, and PDCD1; MZB1, BATF, CXCR6, GZMB, and TIGIT; MZB1, BATF, CXCR6, GZMB, and TNFRSF18; MZB1, BATF, CXCR6, PDCD1, and TIGIT; MZB1, BATF, CXCR6, PDCD1, and TNFRSF18; MZB1, BATF, CXCR6, TIGIT, and TNFRSF18; MZB1, BATF, GZMB, PDCD1, and TIGIT; MZB1, BATF, GZMB, PDCD1, and TNFRSF18; MZB1, BATF, GZMB, TIGIT, and TNFRSF18; MZB1, BATF, PDCD1, TIGIT, and TNFRSF18; MZB1, CXCL13, CXCR6, GZMB, and PDCD1; MZB1, CXCL13, CXCR6, GZMB, and TIGIT; MZB1, CXCL13, CXCR6, GZMB, and TNFRSF18; MZB1, CXCL13, CXCR6, PDCD1, and TIGIT; MZB1, CXCL13, CXCR6, PDCD1, and TNFRSF18; MZB1, CXCL13, CXCR6, TIGIT, and TNFRSF18; MZB1, CXCL13, GZMB, PDCD1, and TIGIT; MZB1, CXCL13, GZMB, PDCD1, and TNFRSF18; MZB1, CXCL13, GZMB, TIGIT, and TNFRSF18; MZB1, CXCL13, PDCD1, TIGIT, and TNFRSF18; MZB1, CXCR6, GZMB, PDCD1, and TIGIT; MZB1, CXCR6, GZMB, PDCD1, and TNFRSF18; MZB1, CXCR6, GZMB, TIGIT, and TNFRSF18; MZB1, CXCR6, PDCD1, TIGIT, and TNFRSF18; MZB1, GZMB, PDCD1, TIGIT, and TNFRSF18; SEC11C, BATF, CXCL13, CXCR6, and GZMB; SEC11C, BATF, CXCL13, CXCR6, and PDCD1; SEC11C, BATF, CXCL13, CXCR6, and TIGIT; SEC11C, BATF, CXCL13, CXCR6, and TNFRSF18; SEC11C, BATF, CXCL13, GZMB, and PDCD1; SEC11C, BATF, CXCL13, GZMB, and TIGIT; SEC11C, BATF, CXCL13, GZMB, and TNFRSF18; SEC11C, BATF, CXCL13, PDCD1, and TIGIT; SEC11C, BATF, CXCL13, PDCD1, and TNFRSF18; SEC11C, BATF, CXCL13, TIGIT, and TNFRSF18; SEC11C, BATF, CXCR6, GZMB, and PDCD1; SEC11C, BATF, CXCR6, GZMB, and TIGIT; SEC11C, BATF, CXCR6, GZMB, and TNFRSF18; SEC11C, BATF, CXCR6, PDCD1, and TIGIT; SEC11C, BATF, CXCR6, PDCD1, and TNFRSF18; SEC11C, BATF, CXCR6, TIGIT, and TNFRSF18; SEC11C, BATF, GZMB, PDCD1, and TIGIT; SEC11C, BATF, GZMB, PDCD1, and TNFRSF18; SEC11C, BATF, GZMB, TIGIT, and TNFRSF18; SEC11C, BATF, PDCD1, TIGIT, and TNFRSF18; SEC11C, CXCL13, CXCR6, GZMB, and PDCD1; SEC11C, CXCL13, CXCR6, GZMB, and TIGIT; SEC11C, CXCL13, CXCR6, GZMB, and TNFRSF18; SEC11C, CXCL13, CXCR6, PDCD1, and TIGIT; SEC11C, CXCL13, CXCR6, PDCD1, and TNFRSF18; SEC11C, CXCL13, CXCR6, TIGIT, and TNFRSF18; SEC11C, CXCL13, GZMB, PDCD1, and TIGIT; SEC11C, CXCL13, GZMB, PDCD1, and TNFRSF18; SEC11C, CXCL13, PDCD1, TIGIT, and TNFRSF18; SEC11C, CXCL13, GZMB, TIGIT, and TNFRSF18; SEC11C, CXCR6, GZMB, PDCD1, and TIGIT; SEC11C, CXCR6, GZMB, PDCD1, and TNFRSF18; SEC11C, CXCR6, GZMB, TIGIT, and TNFRSF18; SEC11C, CXCR6, PDCD1, TIGIT, and TNFRSF18; SEC11C, GZMB, PDCD1, TIGIT, and TNFRSF18; BATF, CXCL13, CXCR6, GZMB, and PDCD1; BATF, CXCL13, CXCR6, GZMB, and TIGIT; BATF, CXCL13, CXCR6, GZMB, and TNFRSF18; BATF, CXCL13, CXCR6, PDCD1, and TIGIT; BATF, CXCL13, CXCR6, PDCD1, and TNFRSF18; BATF, CXCL13, CXCR6, TIGIT, and TNFRSF18; BATF, CXCL13, GZMB, PDCD1, and TIGIT; BATF, CXCL13, GZMB, PDCD1, and TNFRSF18; BATF, CXCL13, GZMB, TIGIT, and TNFRSF18; BATF, CXCL13, PDCD1, TIGIT, and TNFRSF18; BATF, CXCR6, GZMB, PDCD1, and TIGIT; BATF, CXCR6, GZMB, PDCD1, and TNFRSF18; BATF, CXCR6, GZMB, TIGIT, and TNFRSF18; BATF, CXCR6, PDCD1, TIGIT, and TNFRSF18; BATF, GZMB, PDCD1, TIGIT, and TNFRSF18; CXCL13, CXCR6, GZMB, PDCD1, and TIGIT; CXCL13, CXCR6, GZMB, PDCD1, and TNFRSF18; CXCL13, CXCR6, GZMB, TIGIT, and TNFRSF18; CXCL13, CXCR6, PDCD1, TIGIT, and TNFRSF18; CXCL13, GZMB, PDCD1, TIGIT, and TNFRSF18; and CXCR6, GZMB, PDCD1, TIGIT, and TNFRSF18;
   the combination of 6 genes include, without intent of being complete: MZB1, SEC11C, BATF, CXCL13, CXCR6, and GZMB; MZB1, SEC11C, BATF, CXCL13, CXCR6, and PDCD1; MZB1, SEC11C, BATF, CXCL13, CXCR6, and TIGIT; MZB1, SEC11C, BATF, CXCL13, CXCR6, and TNFRSF18; MZB1, SEC11C, BATF, CXCL13, GZMB, and PDCD1; MZB1, SEC11C, BATF, CXCL13, GZMB, and TIGIT; MZB1, SEC11C, BATF, CXCL13, GZMB, and TNFRSF18; MZB1, SEC11C, BATF, CXCL13, PDCD1, and TIGIT; MZB1, SEC11C, BATF, CXCL13, PDCD1, and TNFRSF18; MZB1, SEC11C, BATF, CXCL13, TIGIT, and TNFRSF18; MZB1, SEC11C, CXCL13, CXCR6, GZMB, and PDCD1; MZB1, SEC11C, CXCL13, CXCR6, GZMB, and TIGIT; MZB1, SEC11C, CXCL13, CXCR6, GZMB, and TNFRSF18; MZB1, SEC11C, CXCL13, CXCR6, PDCD1, and TIGIT; MZB1, SEC11C, CXCL13, CXCR6, PDCD1, and TNFRSF18; MZB1, SEC11C, CXCL13, CXCR6, TIGIT, and TNFRSF18; MZB1, SEC11C, CXCR6, GZMB, PDCD1, and TIGIT; MZB1, SEC11C, CXCR6, GZMB, PDCD1, and TNFRSF18; MZB1, SEC11C, CXCR6, GZMB, TIGIT, and TNFRSF18; MZB1, SEC11C, GZMB, PDCD1, TIGIT, and TNFRSF18; MZB1, BATF, CXCL13, CXCR6, GZMB, and PDCD1; MZB1, BATF, CXCL13, CXCR6, GZMB, and TIGIT; MZB1, BATF, CXCL13, CXCR6, GZMB, and TNFRSF18; MZB1, BATF, CXCL13, CXCR6, PDCD1, and TIGIT; MZB1, BATF, CXCL13, CXCR6, PDCD1, and TNFRSF18; MZB1, BATF, CXCL13, CXCR6, TIGIT, and TNFRSF18; MZB1, SEC11C, BATF, CXCL13, CXCR6, and GZMB; MZB1, SEC11C, BATF, CXCL13, CXCR6, and PDCD1; MZB1, BATF, CXCR6, GZMB, PDCD1 and TIGIT; MZB1, BATF, CXCR6, GZMB, PDCD1, and TNFRSF18; MZB1, BATF, CXCR6, GZMB, TIGIT, and TNFRSF18; MZB1, BATF, GZMB, PDCD1, TIGIT, and TNFRSF18; MZB1, CXCL13, CXCR6, GZMB, PDCD1, and TIGIT; MZB1, CXCL13, CXCR6, GZMB, PDCD1, and TNFRSF18; MZB1, CXCL13, CXCR6, GZMB, TIGIT, and TNFRSF18; MZB1, CXCL13, GZMB, PDCD1, TIGIT, and TNFRSF18; MZB1, CXCR6, GZMB, PDCD1, TIGIT, and TNFRSF18; SEC11C, BATF, CXCL13, CXCR6, GZMB, and PDCD1; SEC11C, BATF, CXCL13, CXCR6, GZMB, and TIGIT; SEC11C, BATF, CXCL13, CXCR6, GZMB, and TNFRSF18; SEC11C, BATF, CXCL13, CXCR6, PDCD1, and TIGIT; SEC11C, BATF, CXCL13, CXCR6, PDCD1, and TNFRSF18; SEC11C, BATF, CXCL13, CXCR6, TIGIT, and TNFRSF18; SEC11C, BATF, CXCR6, GZMB, PDCD1, and TIGIT; SEC11C, BATF, CXCR6, GZMB, PDCD1, and TNFRSF18; SEC11C, BATF, CXCR6, GZMB, TIGIT, and TNFRSF18; SEC11C, BATF, GZMB, PDCD1, TIGIT, and TNFRSF18; SEC11C, CXCL13, CXCR6, GZMB, PDCD1, and TIGIT; SEC11C, CXCL13, CXCR6, GZMB, PDCD1, and TNFRSF18; SEC11C, CXCR6, GZMB, PDCD1, TIGIT, and TNFRSF18; BATF, CXCL13, CXCR6, GZMB, PDCD1, and TIGIT; BATF, CXCL13, CXCR6, GZMB, PDCD1, and TNFRSF18; BATF, CXCL13, CXCR6, GZMB, TIGIT, and TNFRSF18; BATF, CXCR6, GZMB, PDCD1, TIGIT, and TNFRSF18; and CXCL13, CXCR6, GZMB, PDCD1, TIGIT, and TNFRSF18;
   the combination of 7 genes include, without intent of being complete: MZB1, SEC11C, BATF, CXCL13, CXCR6, GZMB, and PDCD1; MZB1, SEC11C, BATF, CXCL13, CXCR6, GZMB, and TIGIT; MZB1, SEC11C, BATF, CXCL13, CXCR6, GZMB, and TNFRSF18; MZB1, SEC11C, BATF, CXCL13, CXCR6, PDCD1, and TIGIT; MZB1, SEC11C, BATF, CXCL13, CXCR6, PDCD1, and TNFRSF18; MZB1, SEC11C, BATF, CXCL13, CXCR6, TIGIT, and TNFRSF18; MZB1, SEC11C, CXCL13, CXCR6, GZMB, PDCD1, and TIGIT; MZB1, SEC11C, CXCL13, CXCR6, GZMB, PDCD1, and TNFRSF18; MZB1, SEC11C, CXCL13, CXCR6, GZMB, TIGIT, and TNFRSF18; MZB1, BATF, CXCL13, CXCR6, GZMB, PDCD1, and TIGIT; MZB1, BATF, CXCL13, CXCR6, GZMB, PDCD1, and TNFRSF18; MZB1, BATF, CXCL13, CXCR6, GZMB, TIGIT, and TNFRSF18; MZB1, CXCL13, CXCR6, GZMB, PDCD1, TIGIT, and TNFRSF18; SEC11C, BATF, CXCL13, CXCR6, GZMB, PDCD1, and TIGIT; SEC11C, BATF, CXCL13, CXCR6, GZMB, PDCD1, and TNFRSF18; SEC11C, BATF, CXCL13, CXCR6, GZMB, TIGIT, and TNFRSF18; and BATF, CXCL13, CXCR6, GZMB, PDCD1, TIGIT, and TNFRSF18;
   the combination of 8 genes include, without intent of being complete: MZB1, SEC11C, BATF, CXCL13, CXCR6, GZMB, PDCD1, and TIGIT; MZB1, BATF, CXCL13, CXCR6, GZMB, PDCD1, TIGIT, and TNFRSF18; MZB1, SEC11C, CXCL13, CXCR6, GZMB, PDCD1, TIGIT, and TNFRSF18; MZB1, SEC11C, BATF, CXCR6, GZMB, PDCD1, TIGIT, and TNFRSF18; MZB1, SEC11C, BATF, CXCL13, GZMB, PDCD1, TIGIT, and TNFRSF18; MZB1, SEC11C, BATF, CXCL13, CXCR6, PDCD1, TIGIT, and TNFRSF18; MZB1, SEC11C, BATF, CXCL13, CXCR6, GZMB, TIGIT, and TNFRSF18; MZB1, SEC11C, BATF, CXCL13, CXCR6, GZMB, PDCD1, and TNFRSF18; and SEC11C, BATF, CXCL13, CXCR6, GZMB, PDCD1, TIGIT, and TNFRSF18;
   the combination of 9 genes includes: MZB1, SEC11C, BATF, CXCL13, CXCR6, GZMB, PDCD1, TIGIT, and TNFRSF18.
47) in clarification of the embodiment 25) hereinabove, the combinations of the 2 or more genes include any of the individual gene combinations listed in 46) hereinabove to which the gene HMGB2 is added, and further include the gene combinations MZB1 and HMGB2; SEC11C and HMGB2; BATF and HMGB2; CXCL13 and HMGB2; CXCR6 and HMGB2; GZMB and HMGB2; PDCD1 and HMGB2; TIGIT and HMGB2; TNFRSF18 and HMGB2;
48) in clarification of embodiment 26) hereinabove, the combinations of the 2 or more genes include an individual gene or individual combination of genes of group A in any combination with an individual gene or individual combination of genes of group B, wherein:
   group A consists of an individual gene selected from IGHG1, IGHG2, IGHG3, and IGLC2, and of any individual combination of genes selected from embodiment 38); and
   group B consists of an individual gene selected from MZB1, SEC11C, BATF, and CXCR6, and of any individual combination of genes selected from embodiment 42);
   for clarity the combination is a combination of at least 1 gene of group A and at least 1 gene of group B; or a combination of at least 1 gene of group A further comprising at least 1 gene of group B;
49) in clarification of the embodiment 27) hereinabove, the combinations of the 2 or more genes include any of the individual gene combinations listed in 48) hereinabove to which the gene IGKC is added, and further include the gene combinations IGHG1 and IGKC; IGHG2 and IGKC; IGHG3 and IGKC; IGLC2 and IGKC; MZB1 and IGKC; SEC11C and IGKC; BATF and IGKC; and CXCR6 and IGKC; more in particular, the gene IGKC is added to group A or to group B, more in particular to group B;
50) in clarification of the embodiment 28) hereinabove, the combinations of the 2 or more genes include any of the individual gene combinations listed in 48) hereinabove to which the gene HMGB2 is added, and further include the gene combinations IGHG1 and HMGB2; IGHG2 and HMGB2; IGHG3 and HMGB2; IGLC2 and HMGB2; MZB1 and HMGB2; SEC11C and HMGB2; BATF and HMGB2; and CXCR6 and HMGB2; more in particular, the gene HMGB2 is added to group A or to group B, more in particular to group A;
51) in clarification of the embodiment 29) hereinabove, the combinations of the 2 or more genes include any of the individual gene combinations listed in 48) hereinabove to which the gene IGKC is added, to which the gene HMGB2 is added, or to which the genes IGKC and HMGB2 are added; and further include the gene combinations IGHG1 and IGKC; IGHG2 and IGKC; IGHG3 and IGKC; IGLC2 and IGKC; IGHG1 and HMGB2; IGHG2 and HMGB2; IGHG3 and HMGB2; IGLC2 and HMGB2; MZB1 and IGKC; SEC11C and IGKC; BATF and IGKC; CXCR6 and IGKC; MZB1 and HMGB2; SEC11C and HMGB2; BATF and HMGB2; and CXCR6 and HMGB2; more in particular, the gene IGKC is added to group A or to group B (more in particular to group B), and/or the gene HMGB2 is added to group A or to group B (more in particular to group A);
52) in clarification of embodiment 30) hereinabove, the combinations of the 2 or more genes include an individual gene or individual combination of genes of group A in any combination with an individual gene or individual combination of genes of group C, wherein:
   group A consists of an individual gene selected from IGHG1, IGHG2, IGHG3, and IGLC2, and of any individual combination of genes selected from embodiment 38); and
   group C consists of an individual gene selected from MZB1, SEC11C, BATF, CXCR6, PDCD1, TIGIT, and TNFRSF18, and of any individual combination of genes selected from embodiment 43);
   for clarity the combination is a combination of at least 1 gene of group A and at least 1 gene of group C; or a combination of at least 1 gene of group A further comprising at least 1 gene of group C;
53) in clarification of the embodiment 31) hereinabove, the combinations of the 2 or more genes include any of the individual gene combinations listed in 52) hereinabove to which the gene IGKC is added, and further include the gene combinations IGHG1 and IGKC; IGHG2 and IGKC; IGHG3 and IGKC; IGLC2 and IGKC; MZB1 and IGKC; SEC11C and IGKC; BATF and IGKC; CXCR6 and IGKC; PDCD1 and IGKC; TIGIT and IGKC; and TNFRSF18 and IGKC; more in particular, the gene IGKC is added to group A or to group C, more in particular to group C;
54) in clarification of the embodiment 32) hereinabove, the combinations of the 2 or more genes include any of the individual gene combinations listed in 52) hereinabove to which the gene HMGB2 is added, and further include the gene combinations IGHG1 and HMGB2; IGHG2 and HMGB2; IGHG3 and HMGB2; IGLC2 and HMGB2; MZB1 and HMGB2; SEC11C and HMGB2; BATF and HMGB2; CXCR6 and HMGB2; PDCD1 and HMGB2; TIGIT and HMGB2; and TNFRSF18 and HMGB2; more in particular, the gene HMGB2 is added to group A or to group C, more in particular to group A;
55) in clarification of the embodiment 33) hereinabove, the combinations of the 2 or more genes include any of the individual gene combinations listed in 52) hereinabove to which the gene IGKC is added, to which the gene HMGB2 is added, or to which the genes IGKC and HMGB2 are added; and further include the gene combinations IGHG1 and IGKC; IGHG2 and IGKC; IGHG3 and IGKC; IGLC2 and IGKC; IGHG1 and HMGB2; IGHG2 and HMGB2; IGHG3 and HMGB2; IGLC2 and HMGB2; MZB1 and IGKC; SEC11C and IGKC; BATF and IGKC; CXCR6 and IGKC; PDCD1 and IGKC; TIGIT and IGKC; and TNFRSF18 and IGKC; MZB1 and HMGB2; SEC11C and HMGB2; BATF and HMGB2; CXCR6 and HMGB2; PDCD1 and HMGB2; TIGIT and HMGB2; and TNFRSF18 and HMGB2; more in particular, the gene IGKC is added to group A or to group C (more in particular to group C), and/or the gene HMGB2 is added to group A or to group C (more in particular to group A);
56) in clarification of embodiment 34) hereinabove, the combinations of the 2 or more genes include an individual gene or individual combination of genes of group A in any combination with an individual gene or individual combination of genes of group D, wherein:
   group A consists of an individual gene selected from IGHG1, IGHG2, IGHG3, and IGLC2, and of any individual combination of genes selected from embodiment 38); and
   group D consists of an individual gene selected from MZB1, SEC11C, BATF, CXCL13, CXCR6, GZMB, PDCD1, TIGIT, and TNFRSF18, and of any individual combination of genes selected from embodiment 46);
   for clarity the combination is a combination of at least 1 gene of group A and at least 1 gene of group D; or a combination of at least 1 gene of group A further comprising at least 1 gene of group D;
57) in clarification of the embodiment 35) hereinabove, the combinations of the 2 or more genes include any of the individual gene combinations listed in 56) hereinabove to which the gene IGKC is added, and further include the gene combinations IGHG1 and IGKC; IGHG2 and IGKC; IGHG3 and IGKC; IGLC2 and IGKC; MZB1 and IGKC; SEC11C and IGKC; BATF and IGKC; CXCR6 and IGKC; PDCD1 and IGKC; TIGIT and IGKC; TNFRSF18 and IGKC; GZMB and IGKC; and CXCL13 and IGKC; more in particular, the gene IGKC is added to group A or to group D, more in particular to group D;
58) in clarification of the embodiment 36) hereinabove, the combinations of the 2 or more genes include any of the individual gene combinations listed in 56) hereinabove to which the gene HMGB2 is added, and further include the gene combinations IGHG1 and HMGB2; IGHG2 and HMGB2; IGHG3 and HMGB2; IGLC2 and HMGB2; MZB1 and HMGB2; SEC11C and HMGB2; BATF and HMGB2; CXCR6 and HMGB2; PDCD1 and HMGB2; TIGIT and HMGB2; TNFRSF18 and HMGB2; GZMB and HMGB2; and CXCL13 and HMGB2; more in particular, the gene HMGB2 is added to group A or to group D, more in particular to group A;
59) in clarification of the embodiment 37) hereinabove, the combinations of the 2 or more genes include any of the individual gene combinations listed in 56) hereinabove to which the gene IGKC is added, to which the gene HMGB2 is added, or to which the genes IGKC and HMGB2 are added; and further include the gene combinations IGHG1 and IGKC; IGHG2 and IGKC; IGHG3 and IGKC; IGLC2 and IGKC; IGHG1 and HMGB2; IGHG2 and HMGB2; IGHG3 and HMGB2; IGLC2 and HMGB2; MZB1 and IGKC; SEC11C and IGKC; BATF and IGKC; CXCR6 and IGKC; PDCD1 and IGKC; TIGIT and IGKC; and TNFRSF18 and IGKC; GZMB and IGKC; and CXCL13 and IGKC; MZB1 and HMGB2; SEC11C and HMGB2; BATF and HMGB2; and CXCR6 and HMGB2; PDCD1 and HMGB2; TIGIT and HMGB2; and TNFRSF18 and HMGB2; GZMB and HMGB2; and CXCL13 and HMGB2; more in particular, the gene IGKC is added to group A or to group D (more in particular to group D), and/or the gene HMGB2 is added to group A or to group D (more in particular to group A);
60) in one embodiment, 1, 2 or more genes (2, 3, 4, 5, or 6) selected from the group (consisting of) MZB1, SEC11C, BATF, CXCR6, TIGIT, and TNFRSF18;
61) in clarification of embodiment 60) hereinabove,
   the combination of 2 genes include, without intent of being complete: MZB1 and SEC11C; MZB1 and BATF; MZB1 and CXCR6; MZB1 and TIGIT; MZB1 and TNFRSF18; SEC11C and BATF; SEC11C and CXCR6; SEC11C and TIGIT; SEC11C and TNFRSF18; BATF and CXCR6; BATF and TIGIT; BATF and TNFRSF18; CXCR6 and TIGIT; CXCR6 and TNFRSF18; TIGIT and TNFRSF18;
   the combination of 3 genes include, without intent of being complete: MZB1, SEC11C and BATF; MZB1, SEC11C and CXCR6; MZB1, SEC11C and TIGIT; MZB1, SEC11C and TNFRSF18; MZB1, BATF and CXCR6; MZB1, BATF and TIGIT; MZB1, BATF and TNFRSF18; MZB1, CXCR6 and TIGIT; MZB1, CXCR6 and TNFRSF18; MZB1, TIGIT and TNFRSF18; SEC11C, BATF and CXCR6; SEC11C, BATF and TIGIT; SEC11C, BATF and TNFRSF18; SEC11C, CXCR6 and TIGIT; SEC11C, CXCR6 and TNFRSF18; SEC11C, TIGIT and TNFRSF18; BATF, CXCR6 and TIGIT; BATF, CXCR6 and TNFRSF18; BATF, TIGIT and TNFRSF18; CXCR6, TIGIT and TNFRSF18;
   the combination of 4 genes include, without intent of being complete: MZB1, SEC11C, BATF and CXCR6; MZB1, SEC11C, BATF and TIGIT; MZB1, SEC11C, BATF and TNFRSF18; MZB1, BATF, CXCR6 and TIGIT; MZB1, BATF, CXCR6 and TNFRSF18; MZB1, SEC11C, CXCR6 and TIGIT; MZB1, SEC11C, CXCR6 and TNFRSF18; MZB1, SEC11C, TIGIT and TNFRSF18; MZB1, CXCR6, TIGIT and TNFRSF18; MZB1, BATF, TIGIT and TNFRSF18; SEC11C, BATF, CXCR6 and TIGIT; SEC11C, BATF, CXCR6 and TNFRSF18; SEC11C, CXCR6, TIGIT and TNFRSF18; SEC11C, BATF, TIGIT and TNFRSF18; BATF, CXCR6, TIGIT and TNFRSF18;
   the combination of 5 genes include, without intent of being complete: MZB1, SEC11C, BATF, CXCR6 and TIGIT; MZB1, SEC11C, BATF, CXCR6 and TNFRSF18; MZB1, BATF, CXCR6, TIGIT and TNFRSF18; MZB1, SEC11C, CXCR6, TIGIT and TNFRSF18; MZB1, SEC11C, BATF, TIGIT and TNFRSF18; SEC11C, BATF, CXCR6, TIGIT and TNFRSF18;
   the combination of 6 genes includes: MZB1, SEC11C, BATF, CXCR6, TIGIT and TNFRSF18;
62) a combination of 2 or more genes including an individual gene or individual combination of genes of group A in any combination with an individual gene or individual combination of genes of group E, wherein:
   group A consists of an individual gene selected from IGHG1, IGHG2, IGHG3, and IGLC2, and of any individual combination of genes selected from embodiment 38); and
   group E consists of an individual gene selected from MZB1, SEC11C, BATF, CXCR6, TIGIT, and TNFRSF18, and of any individual combination of genes selected from embodiment 61);
   for clarity the combination is a combination of at least 1 gene of group A and at least 1 gene of group E; or a combination of at least 1 gene of group A further comprising at least 1 gene of group E;
63) in clarification of the embodiment 62) hereinabove, the combinations of the 2 or more genes include any of the individual gene combinations listed in 62) hereinabove to which the gene IGKC is added, and further include the gene combinations IGHG1 and IGKC; IGHG2 and IGKC; IGHG3 and IGKC; IGLC2 and IGKC; MZB1 and IGKC; SEC11C and IGKC; BATF and IGKC; CXCR6 and IGKC; TIGIT and IGKC; and TNFRSF18 and IGKC; more in particular, the gene IGKC is added to group A or to group E, more in particular to group E;
64) in clarification of the embodiment 62) hereinabove, the combinations of the 2 or more genes include any of the individual gene combinations listed in 62) hereinabove to which the gene HMGB2 is added, and further include the gene combinations IGHG1 and HMGB2; IGHG2 and HMGB2; IGHG3 and HMGB2; IGLC2 and HMGB2; MZB1 and HMGB2; SEC11C and HMGB2; BATF and HMGB2; CXCR6 and HMGB2; TIGIT and HMGB2; and TNFRSF18 and HMGB2; more in particular, the gene HMGB2 is added to group A or to group E, more in particular to group A;
65) in clarification of the embodiment 62) hereinabove, the combinations of the 2 or more genes include any of the individual gene combinations listed in 62) hereinabove to which the gene IGKC is added, to which the gene HMGB2 is added, or to which the genes IGKC and HMGB2 are added; and further include the gene combinations IGHG1 and IGKC; IGHG2 and IGKC; IGHG3 and IGKC; IGLC2 and IGKC; IGHG1 and HMGB2; IGHG2 and HMGB2; IGHG3 and HMGB2; IGLC2 and HMGB2; MZB1 and IGKC; SEC11C and IGKC; BATF and IGKC; CXCR6 and IGKC; TIGIT and IGKC; and TNFRSF18 and IGKC; MZB1 and HMGB2; SEC11C and HMGB2; BATF and HMGB2; CXCR6 and HMGB2; TIGIT and HMGB2; and TNFRSF18 and HMGB2; more in particular, the gene IGKC is added to group A or to group E (more in particular to group E), and/or the gene HMGB2 is added to group A or to group E (more in particular to group A);
66) in one embodiment: in any of the preceding embodiments referring to combinations of 2 or more genes optionally including the gene IGHG1, the combination is effectively (not optionally) including the gene IGHG1, thus being a combination of 2 or more genes of which one of the genes is IGHG1;
67) in one embodiment: in any of the preceding embodiments referring to combinations of 2 or more genes optionally including the gene IGHG2, the combination is effectively (not optionally) including the gene IGHG2, thus being a combination of 2 or more genes of which one of the genes is IGHG2;
68) in one embodiment: in any of the preceding embodiments referring to combinations of 2 or more genes optionally including the gene IGHG3, the combination is effectively (not optionally) including the gene IGHG3, thus being a combination of 2 or more genes of which one of the genes is IGHG3;
69) in one embodiment: in any of the preceding embodiments referring to combinations of 2 or more genes optionally including the gene IGLC2, the combination is effectively (not optionally) including the gene IGLC2, thus being a combination of 2 or more genes of which one of the genes is IGLC2;
70) in one embodiment: in any of the preceding embodiments referring to combinations of 2 or more genes optionally including the gene IGKC, the combination is effectively (not optionally) including the gene IGKC, thus being a combination of 2 or more genes of which one of the genes is IGKC;
71) in one embodiment: in any of the preceding embodiments referring to combinations of 2 or more genes optionally including the gene IGHG1 or the gene IGHG2, the combination is effectively (not optionally) including the genes IGHG1 and IGHG2, thus being a combination of 2 or more genes of which two genes are IGHG1 and IGHG2;
72) in one embodiment: in any of the preceding embodiments referring to combinations of 2 or more genes optionally including the gene IGHG1 or the gene IGHG3, the combination is effectively (not optionally) including the genes IGHG1 and IGHG3, thus being a combination of 2 or more genes of which two genes are IGHG1 and IGHG3;
73) in one embodiment: in any of the preceding embodiments referring to combinations of 2 or more genes optionally including the gene IGHG1 or the gene IGLC2, the combination is effectively (not optionally) including the genes IGHG1 and IGLC2, thus being a combination of 2 or more genes of which two genes are IGHG1 and IGLC2;
74) in one embodiment: in any of the preceding embodiments referring to combinations of 2 or more genes optionally including the gene IGHG1 or the gene IGKC, the combination is effectively (not optionally) including the genes IGHG1 and IGKC, thus being a combination of 2 or more genes of which two genes are IGHG1 and IGKC;
75) in one embodiment: in any of the preceding embodiments referring to combinations of 2 or more genes optionally including the gene IGHG2 or the gene IGHG3, the combination is effectively (not optionally) including the genes IGHG2 and IGHG3, thus being a combination of 2 or more genes of which two genes are IGHG2 and IGHG3;
76) in one embodiment: in any of the preceding embodiments referring to combinations of 2 or more genes optionally including the gene IGHG2 or the gene IGLC2, the combination is effectively (not optionally) including the genes IGHG2 and IGLC2, thus being a combination of 2 or more genes of which two genes are IGHG2 and IGLC2;
77) in one embodiment: in any of the preceding embodiments referring to combinations of 2 or more genes optionally including the gene IGHG2 or the gene IGKC, the combination is effectively (not optionally) including the genes IGHG2 and IGKC, thus being a combination of 2 or more genes of which two genes are IGHG2 and IGKC;
78) in one embodiment: in any of the preceding embodiments referring to combinations of 2 or more genes optionally including the gene IGHG3 or the gene IGLC2, the combination is effectively (not optionally) including the genes IGHG3 and IGLC2, thus being a combination of 2 or more genes of which two genes are IGHG3 and IGLC2;
79) in one embodiment: in any of the preceding embodiments referring to combinations of 2 or more genes optionally including the gene IGHG3 or the gene IGKC, the combination is effectively (not optionally) including the genes IGHG3 and IGKC, thus being a combination of 2 or more genes of which two genes are IGHG3 and IGKC;
80) in one embodiment: in any of the preceding embodiments referring to combinations of 2 or more genes optionally including the gene IGLC2 or the gene IGKC, the combination is effectively (not optionally) including the genes IGLC2 and IGKC, thus being a combination of 2 or more genes of which two genes are IGLC2 and IGKC.

In particular, embodiments 1) to 15) hereinabove refer to single genes. As outlined in the Examples herein, expression levels of the single genes indeed all individually are strong predictors of the outcome of/response to the immunotherapy or immunogenic therapy. Furthermore, expression levels of the single genes indeed all individually are strong predictors of the outcome of/response to the immunotherapy or immunogenic therapy of breast cancer patients, but not of melanoma patients. In other words, by means of their differential expression in responders to immunotherapy or immunogenic therapy versus/compared to in non-responders to immunotherapy or immunogenic therapy, each of the single genes individually is a strong predictors of the outcome of/response to the immunotherapy or immunogenic therapy of breast cancer patients, but not of melanoma patients. In a particular embodiment, the expression of the one or more biomarker gene is, when determined by quantifying the corresponding mRNA levels, determined in bulk mRNA obtained from the (biological) sample obtained from the subject having breast cancer. Whereas (immune) cell subtypes may individually have a specific set of differentially expressed genes predictive of response to immunotherapy or immunogenic therapy, it is not a priori possible to conclude that cell-subtype specific differential expression of genes is still observable as differential expression in bulk mRNA which does not make distinction between cell subtypes. Indeed, differential expression of a gene X in e.g. CD4+ T-cells specifically may be completely diluted and go unnoticed (i.e. no differential expression) when determined in the whole of (the mRNA obtained from) the biological sample.

### Immunotherapy and Immunogenic Therapy

Immunotherapy in general is defined as a treatment that uses the body's own immune system to help fight a disease, more specifically breast cancer in the context of the current invention. In general, immunotherapeutic treatment as used herein refers to the reactivation and/or stimulation and/or reconstitution of the immune response of a mammal towards a condition such as a tumor, cancer or neoplasm evading and/or escaping and/or suppressing normal immune surveillance. The reactivation and/or stimulation and/or reconstitution of the immune response of a mammal in turn in part results in an increase in elimination of tumorous, cancerous or neoplastic cells by the mammal's immune system (anticancer, antitumor or anti-neoplasm immune response; adaptive immune response to the tumor, cancer or neoplasm). Immunotherapeutic agents of particular interest non-exhaustively include immune checkpoint inhibitors (such as anti-PD-1, anti-PD-L1 or anti-CTLA-4 antibodies), bispecific antibodies bridging a cancer cell and an immune cell, and dendritic cell vaccines. Immunotherapy is a promising new area of cancer therapeutics and several immunotherapies are being evaluated preclinically as well as in clinical trials and have demonstrated promising activity (Callahan et al. 2013, J Leukoc Biol 94:41-53; Page et al. 2014, Annu Rev Med 65:185-202). However, not all the patients are sensitive to immune checkpoint blockade and sometimes PD-1 or PD-L1 blocking antibodies accelerate tumor progression. An overview of clinical developments in the field of immune checkpoint therapy is given by Fan et al. 2019 (Oncology Reports 41:3-14). Monoclonal antibodies targeting and inhibiting PD-1 include pembrolizumab, nivolumab, and cemiplimab. Monoclonal antibodies targeting and inhibiting PD-L1 include atezolizumab, avelumab, and durvalumab. Monoclonal antibodies targeting and inhibiting CTLA-4 include ipilimumab. Combinatorial cancer treatments that include chemotherapies can achieve higher rates of disease control by impinging on distinct elements of tumor biology to obtain synergistic antitumor effects. It is now accepted that certain chemotherapies can increase tumor immunity by inducing immunogenic cell death and by promoting escape in cancer immunoediting, such therapies are therefore called immunogenic therapies as they provoke an immunogenic response. Drug moieties known to induce immunogenic cell death include bleomycin, bortezomib, cyclophosphamide, doxorubicin, epirubicin, idarubicin, mafosfamide, mitoxantrone, oxaliplatin, and patupilone (Bezu et al. 2015, Front Immunol 6:187). Other forms of immunotherapy include chimeric antigen receptor (CAR) T-cell therapy in which allogenic T-cells are adapted to recognize a tumoral neo-antigen and oncolytic viruses preferentially infecting and killing cancer cells. Treatment with RNA, e.g. encoding MLKL, is a further means of provoking an immunogenic response (Van Hoecke et al. 2018, Nat Commun 9:3417), as well as vaccination with neo-epitopes (Brennick et al. 2017, Immunotherapy 9:361-371). Information on PD1, PD-L1 and CTLA4 is included hereafter in a format similar format as for the individual biomarkers hereinabove.

PD1 Aliases of PD1 provided in GeneCards® include PDCD1; Programmed Cell Death 1; Systemic Lupus Erythematosus Susceptibility 2; PD-1; CD279; HPD-1; SLEB2; and HPD-L. The genomic locations for the PDCD1 gene are chr2:241,849,881-241,858,908 (in GRCh38/hg38) and chr2:242,792,033-242,801,060 (in GRCh37/hg19). The GenBank reference PD1 mRNA sequence is known under accession no. NM_005018.3. Approved PD1-inhibiting antibodies include nivolumab, pembrolizumab, and cemiplimab; PD1-inhibiting antibodies under development include CT-011 (pidilizumab) and therapy with PD1-inhibiting antibodies is referred to herein as α-PD-1 therapy or α-PD1 therapy. PD1 siRNA and shRNA products are available through e.g. Origene.

### PD-L1

Aliases of PD-L1 provided in GeneCards® include CD274, Programmed Cell Death 1 Ligand 1, B7 Homolog 1, B71H1, PDL1, PDCD1 Ligand 1, PDCD1LG1, PDCD1L1, HPD-L1, B7-H1, B7-H, and Programmed Death Ligand 1. The genomic locations for the PDCD1 gene are chr9:5,450,503-5,470,567 (in GRCh38/hg38) and chr9:5,450,503-5,470,567 (in GRCh37/hg19). The GenBank reference PD1 mRNA sequence is known under accession no. NM_001267706.1, NM_001314029.2 and NM_014143.4. Approved PD-L1-inhibiting antibodies include atezolizumab, avelumab, and durvalumab. PD-L1 siRNA and shRNA products are available through e.g. Origene.

### CTLA4

Aliases of CTLA4 provided in GeneCards® include Cytotoxic T-Lymphocyte Associated Protein 4; CTLA-4; CD152; Insulin-Dependent Diabetes Mellitus 12; Cytotoxic T-Lymphocyte Protein 4; Celiac Disease 3; GSE; Ligand And Transmembrane Spliced Cytotoxic T Lymphocyte Associated Antigen 4; Cytotoxic T Lymphocyte Associated Antigen 4 Short Spliced Form; Cytotoxic T-Lymphocyte-Associated Serine Esterase-4; Cytotoxic T-Lymphocyte-Associated Antigen 4; CELIAC3; IDDM12; ALPS5; and GRD4. The genomic locations for the CTLA4 gene are chr2:203,867,771-203,873,965 (in GRCh38/hg38) and chr2:204,732,509-204,738,683 (in GRCh37/hg19). The GenBank reference CTLA4 mRNA sequences are known under accession nos. NM_001037631.3 and NM_005214.5. Approved CTLA4-inhibiting antibodies include ipilumab; CTLA4-inhibiting antibodies under development include tremelimumab. CTLA4 siRNA and shRNA products are available through e.g. Origene.

### Gene Expression and Quantification of Gene Expression

The term “level of expression” or “expression level” generally refers to the amount of an expressed biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) are also regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs, long non-coding RNA, microRNA or miRNA).

“Increased/higher expression,” “increased/higher expression level,” “increased/higher levels,” “elevated expression,” “elevated expression levels,” or “elevated levels” refers to an increased/higher expression or to increased/higher levels of a biomarker in an individual relative to a suitable control or standard. The term “detection” includes any means of detecting, including direct and indirect detection. The term “biomarker” as used herein refers to an indicator molecule or set of molecules (e.g., predictive, diagnostic, and/or prognostic indicator), which can be detected in a sample. The biomarker may be a predictive biomarker and serve as an indicator of the likelihood of sensitivity or benefit to therapeutic treatment of a patient having a particular disease or disorder (e.g., a proliferative cell disorder (e.g., cancer)) to treatment. Biomarkers include, but are not limited to, polynucleotides (e.g., DNA and/or RNA (e.g., mRNA)), polynucleotide copy number alterations (e.g., DNA copy numbers), polypeptides, polypeptide and polynucleotide modifications (e.g., post-translational modifications, nucleotide substitutions, nucleotide insertions or deletions (indels)), carbohydrates, and/or glycolipid-based molecular markers. In some embodiments, a biomarker is a gene. The “amount” or “level” of a biomarker, as used herein, is a detectable level in a biological sample. These can be measured by methods known to one skilled in the art and also disclosed herein.

In first instance, methodologies for determining gene expression by means of determining transcript levels, also referred to as transcriptome analysis or analysis of the transcriptome, is described in more detail. Any such gene detection or gene expression detection method is starting from an analyte nucleic acid (i.e. the nucleic acid of interest (which does not necessarily need to be the whole nucleic acid of interest, parts of such nucleic acids can suffice for determining expression) and of which the amount is to be determined) and may be defined as comprising one or more steps of, for instance,
a step of isolating RNA from a (biological) sample (wherein a fraction of the isolated RNA is the analyte strand);
a step of reverse transcribing the RNA obtained from the biological sample into DNA;
a step of amplifying the isolated DNA; and/or
a step of quantifying the isolated RNA, the DNA obtained after reverse transcription, or the amplified DNA.

In case an amplified DNA is quantified, this quantification step can be performed concurrent with the amplification of the DNA, or is performed after the amplification of the DNA.

The quantification of gene expression or the determination of gene expression levels may be based on at least one of an amplification reaction, a sequencing reaction, a melting reaction, a hybridization reaction or a reverse hybridization reaction.

The invention covers methods which include detection/quantification of nucleic acids corresponding to one or more biomarkers as defined herein. In any of these methods the detection can comprise a step such as a nucleic acid amplification reaction, a nucleic acid sequencing reaction, a melting reaction, a hybridization reaction to a nucleic acid, or a reverse hybridization reaction to a nucleic acid, or a combination of such steps.

Often one or more artificial, man-made, or non-naturally occurring oligonucleotide is used in such method. In particular, such oligonucleotides can comprise besides ribonucleic acid monomers or deoxyribonucleic acid monomers: one or more modified nucleotide bases, one or more modified nucleotide sugars, one or more labelled nucleotides, one or more peptide nucleic acid monomers, one or more locked nucleic acid monomers, the backbone of such oligonucleotide can be modified, and/or non-glycosidic bonds may link two adjacent nucleotides. Such oligonucleotides may further comprise a modification for attachment to a solid support, e.g., an amine-, thiol-, 3-′propanolamine or acrydite-modification of the oligonucleotide, or may comprise the addition of a homopolymeric tail (for instance an oligo(dT)-tail added enzymatically via a terminal transferase enzyme or added synthetically) to the oligonucleotide. If said homopolymeric tail is positioned at the 3′-terminus of the oligonucleotide or if any other 3′-terminal modification preventing enzymatic extension is incorporated in the oligonucleotide, the priming capacity of the oligonucleotide can be decreased or abolished. Such oligonucleotides may also comprise a hairpin structure at either end. Terminal extension of such oligonucleotide may be useful for, e.g., specifically hybridizing with another nucleic acid molecule (e.g. when functioning as capture probe), and/or for facilitating attachment of said oligonucleotide to a solid support, and/or for modification of said tailed oligonucleotide by an enzyme, ribozyme or DNAzyme. Such oligonucleotides may be modified in order to detect (the levels of) a target nucleotide sequence and/or to facilitate in any way such detection. Such modifications include labelling with a single label, with two different labels (for instance two fluorophores or one fluorophore and one quencher), the attachment of a different ‘universal’ tail to two probes or primers hybridizing adjacent or in close proximity to each other with the target nucleotide sequence, the incorporation of a target-specific sequence in a hairpin oligonucleotide (for instance Molecular Beacon-type primer), the tailing of such a hairpin oligonucleotide with a ‘universal’ tail (for instance Sunrise-type probe and Amplifluor™-type primer). A special type of hairpin oligonucleotide incorporates in the hairpin a sequence capable of hybridizing to part of the newly amplified target DNA. Amplification of the hairpin is prevented by the incorporation of a blocking non-amplifiable monomer (such as hexethylene glycol). A fluorescent signal is generated after opening of the hairpin due to hybridization of the hairpin loop with the amplified target DNA. This type of hairpin oligonucleotide is known as scorpion primers (Whitcombe et al. 1999, Nat Biotechnol 17:804-807). Another special type of oligonucleotide is a padlock oligonucleotide (or circularizable, open circle, or C-oligonucleotide) that are used in RCA (rolling circle amplification). Such oligonucleotides may also comprise a 3′-terminal mismatching nucleotide and/or, optionally, a 3′-proximal mismatching nucleotide, which can be particularly useful for performing polymorphism-specific PCR and LCR (ligase chain reaction) or any modification of PCR or LCR. Such oligonucleotide may can comprise or consist of at least and/or comprise or consist of up to 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 or more contiguous nucleotides.

The analyte nucleic acid, in particular the analyte nucleic acid of a biomarker of interest can be any type of nucleic acid, which will be dependent on the manipulation steps (such as isolation and/or purification and/or duplication, multiplication or amplification) applied to the nucleic acid of the gene of interest in the biological sample; as such it can be DNA, RNA, cDNA, may comprise modified nucleotides, or may be hybrids of DNA and/or RNA and/or modified nucleotides, and can be single- or double-stranded or may be a triplex-forming nucleic acid.

The artificial, man-made, non-naturally occurring oligonucleotide(s) as applied in the above detection methods can be probe(s) or a primer(s), or a combination of both.

A probe capable of specifically hybridizing with a target nucleic acid is an oligonucleotide mainly hybridizing to one specific nucleic acid sequence in a mixture of many different nucleic acid sequences. Specific hybridization is meant to result, upon detection of the specifically formed hybrids, in a signal-to-noise ratio (wherein the signal represents specific hybridization and the noise represents unspecific hybridization) sufficiently high to enable unambiguous detection of said specific hybrids. In a specific case specific hybridization allows discrimination of up to a single nucleotide mismatch between the probe and the target nucleic acids. Conditions allowing specific hybridization generally are stringent but can obviously be varied depending on the complexity (size, GC-content, overall identity, etc.) of the probe(s) and/or target nucleic acid molecules. Specificity of a probe in hybridizing with a nucleic acid can be improved by introducing modified nucleotides in said probe.

A primer capable of directing specific amplification of a target nucleic acid is the at least one oligonucleotide in a nucleic acid amplification reaction mixture that is required to obtain specific amplification of a target nucleic acid. Nucleic acid amplification can be linear or exponential and can result in an amplified single nucleic acid of a single- or double-stranded nucleic acid or can result in both strands of a double-stranded nucleic acid. Specificity of a primer in directing amplification of a nucleic acid can be improved by introducing modified nucleotides in said primer. The fact that a primer does not have to match exactly with the corresponding template or target sequence to warrant specific amplification of said template or target sequence is amply documented in literature (for instance: Kwok et al. 1990, Nucl Acids Res 18:999-1005. Primers as short as 8 nucleotides in length have been applied successfully in directing specific amplification of a target nucleic acid molecule (e.g. Majzoub et al. 1983, J Biol Chem 258:14061-14064).

A nucleotide is meant to include any naturally occurring nucleotide as well as any modified nucleotide wherein said modification can occur in the structure of the nucleotide base (modification relative to A, T, G, C, or U) and/or in the structure of the nucleotide sugar (modification relative to ribose or deoxyribose). Any of the modifications can be introduced in a nucleic acid or oligonucleotide to increase/decrease stability and/or reactivity of the nucleic acid or oligonucleotide and/or for other purposes such as labelling of the nucleic acid or oligonucleotide. Modified nucleotides include phosphorothioates, alkylphosphorothioates, methylphosphonate, phosphoramidate, peptide nucleic acid monomers and locked nucleic acid monomers, cyclic nucleotides, and labelled nucleotides (i.e. nucleotides conjugated to a label which can be isotopic (<32>P, <35>S, etc.) or non-isotopic (biotin, digoxigenin, phosphorescent labels, fluorescent labels, fluorescence quenching moiety, etc.)). Other modifications are described higher (see description on oligonucleotides).

Nucleotide acid amplification is meant to include all methods resulting in multiplication of the number of a target nucleic acid. Nucleotide sequence amplification methods include the polymerase chain reaction (PCR; DNA amplification), strand displacement amplification (SDA; DNA amplification), transcription-based amplification system (TAS; RNA amplification), self-sustained sequence replication (3SR; RNA amplification), nucleic acid sequence-based amplification (NASBA; RNA amplification), transcription-mediated amplification (TMA; RNA amplification), Qbeta-replicase-mediated amplification and run-off transcription. During amplification, the amplified products can be conveniently labeled either using labeled primers or by incorporating labeled nucleotides.

The most widely spread nucleotide sequence amplification technique is PCR. The target DNA is exponentially amplified. Many methods rely on PCR including AFLP (amplified fragment length polymorphism), IRS-PCR (interspersed repetitive sequence PCR), iPCR (inverse PCR), RAPD (rapid amplification of polymorphic DNA), RT-PCR (reverse transcription PCR) and real-time PCR. RT-PCR can be performed with a single thermostable enzyme having both reverse transcriptase and DNA polymerase activity (Myers et al. 1991, Biochem 30:7661-7666). Alternatively, a single tube-reaction with two enzymes (reverse transcriptase and thermostable DNA polymerase) is possible (Cusi et al. 1994, Biotechniques 17:1034-1036).

Solid phases, solid matrices or solid supports on which molecules, e.g., nucleic acids, analyte nucleic acids and/or oligonucleotides as described hereinabove, may be bound (or captured, absorbed, adsorbed, linked, coated, immobilized; covalently or non-covalently) comprise beads or the wells or cups of microtiter plates, or may be in other forms, such as solid or hollow rods or pipettes, particles, e.g., from 0.1 μm to 5 mm in diameter (e.g. “latex” particles, protein particles, or any other synthetic or natural particulate material), microspheres or beads (e.g. protein A beads, magnetic beads). A solid phase may be of a plastic or polymeric material such as nitrocellulose, polyvinyl chloride, polystyrene, polyamide, polyvinylidene fluoride or other synthetic polymers. Other solid phases include membranes, sheets, strips, films and coatings of any porous, fibrous or bibulous material such as nylon, polyvinyl chloride or another synthetic polymer, a natural polymer (or a derivative thereof) such as cellulose (or a derivative thereof such as cellulose acetate or nitrocellulose). Fibers or slides of glass, fused silica or quartz are other examples of solid supports. Paper, e.g., diazotized paper may also be applied as solid phase. Clearly, molecules such as nucleic acids, analyte nucleic acids and/or oligonucleotides as described hereinabove, may be bound, captured, absorbed, adsorbed, linked or coated to any solid phase suitable for use in hybridization assay (irrespective of the format, for instance capture assay, reverse hybridization assay, or dynamic allele-specific hybridization (DASH)). Said molecules, such as nucleic acids, analyte nucleic acids and/or oligonucleotides as described hereinabove, can be present on a solid phase in defined zones such as spots or lines. Such solid phases may be incorporated in a component such as a cartridge of e.g. an assay device. Any of the solid phases described above can be developed, e.g. automatically developed in an assay device.

Quantification of amplified DNA can be performed concurrent with or during the amplification. Techniques include real-time PCR or (semi-)quantitative polymerase chain reaction (qPCR). One common method includes measurement of a non-sequence specific fluorescent dye (e.g. SYBR Green) intercalating in any double-stranded DNA. Quantification of multiple amplicons with different melting points can be followed simultaneously by means of following or analyzing the melting reaction (melting curve analysis or melt curve analysis; which can be performed at high resolution, see, e.g. Wittwer et al. 2003, Clin Chem 843-860; an alternative method is denaturing gel gradient electrophoresis, DGGE; both methods were compared in e.g. Tindall et al. 2009, Hum Mutat 30:857-859).

Another common method includes measurement of sequence-specific labelled probe bound to its complementary sequence; such probe also carries a quencher and the label is only measurable upon exonucleolytic release from the probe (hydrolysis probes such as TaqMan probes) or upon hybridization with the target sequence (hairpin probes such as molecular beacons which carry an internally quenched fluorophore whose fluorescence is restored upon unfolding the hairpin). This latter method allows for multiplexing by e.g. using mixtures of probes each tagged with a different label e.g. fluorescing at a different wavelength.

Exciton-controlled hybridization-sensitive fluorescent oligonucleotide (ECHO) probes also allow for multiplexing. The hybridization-sensitive fluorescence emission of ECHO probes and the further modification of probes have made possible multicolor RNA imaging in living cells and facile detection of gene polymorphisms (Okamoto 2011, Chem Soc Rev, 40:5815-5828).

Other methods of quantifying expression include SAGE (Serial Analysis of Gene Expression) and MPSS (Massively Parallel Signature Sequencing), each involving reverse-transcription of RNA.

With “assaying” or “determining” or “detecting” and the like (e.g. assessing, measuring) is meant that a biological sample, suspected of comprising a target nucleic acid (such as a nucleic acid of a biomarker of interest as described herein), is processed as to generate a readable signal in case the target nucleic acid is actually present in the biological sample. Such processing may include, as described above, a step of producing an analyte nucleic acid. Simple detection of a produced readable signal indicates the presence of a target or analyte nucleic acid in the biological sample. When in addition the amplitude of the produced readable signal is determined, this allows for quantification of levels of a target or analyte nucleic acid as present in a biological sample.

In particular, the readable signal may be a signal-to-noise ratio (wherein the signal represents specific detection and the noise represents unspecific detection) of an assay optimized to yield signal-to-noise ratios sufficiently high to enable unambiguous detection and/or quantification of the target nucleic acid. The noise signal, or background signal, can be determined e.g. on biological samples not comprising the target or analyte nucleic acid of interest, e.g. control samples, or comprising the required reference level of the target or analyte nucleic acid of interest, e.g. reference samples. Such noise or background signal may also serve as comparator value for determining an increase or decrease of the level of a target or analyte nucleic acid in the biological sample, e.g. in a biological sample taken from a subject suffering from a disease or disorder, further e.g. before start of a treatment and during treatment.

The readable signal may be produced with all required components in solution or may be produced with some of the required components in solution and some bound to a solid support. Said signals include, e.g., fluorescent signals, (chemi)luminescent signals, phosphorescence signals, radiation signals, light or color signals, optical density signals, hybridization signals, mass spectrometric signals, spectrometric signals, chromatographic signals, electric signals, electronic signals, electrophoretic signals, real-time PCR signals, PCR signals, LCR signals, Invader-assay signals, sequencing signals (by any method such as Sanger dideoxy sequencing, pyrosequencing, 454 sequencing, single-base extension sequencing, sequencing by ligation, sequencing by synthesis, “next-generation” sequencing (NGS) (van Dijk et al. 2014, Trends Genet 30:418-426)), nanopore sequencing, melting curve signals etc. An assay may be run automatically or semi-automatically in an assay device. In view of its relatively low costs compared to e.g. very costly cancer therapies, NGS is finding its way to routine clinical care (Ratner 2018, Nature Biotechnol 36:484).

Specific hybridization of an oligonucleotide (whether or not comprising one or more modified nucleotides) to its target sequence is to be understood to occur under stringent conditions as generally known in the art (e.g. Sambrook et al. 1989. Molecular Cloning. A laboratory manual. CSHL Press). However, depending to the hybridization solution (SSC, SSPE, etc.), oligonucleotides should be hybridized at their appropriate temperature in order to attain sufficient specificity. In order to allow hybridization to occur, the target nucleic acid molecules are generally thermally, chemically (e.g. by NaOH) or electrochemically denatured to melt a double strand into two single strands and/or to remove hairpins or other secondary structures from single stranded nucleic acids. The stringency of hybridization is influenced by conditions such as temperature, salt concentration and hybridization buffer composition. High stringency conditions for hybridization include high temperature and/or low salt concentration (salts include NaCl and Na3-citrate) and/or the inclusion of formamide in the hybridization buffer and/or lowering the concentration of compounds such as SDS (detergent) in the hybridization buffer and/or exclusion of compounds such as dextran sulfate or polyethylene glycol (promoting molecular crowding) from the hybridization buffer. Conventional hybridization conditions are described in e.g. Sambrook et al. 1989 (Molecular Cloning. A laboratory manual. CSHL Press) but the skilled craftsman will appreciate that numerous different hybridization conditions can be designed in function of the known or the expected homology and/or length of the nucleic acid sequence. Generally, for hybridizations with DNA oligonucleotides without formamide, a temperature of 68 DEG C., and for hybridization with formamide, 50% (v/v), a temperature of 42 DEG C. is recommended. For hybridizations with oligonucleotides, the optimal conditions (formamide concentration and/or temperature) depend on the length and base composition of the probe and must be determined individually. In general, optimal hybridization for oligonucleotides of about 10 to 50 bases in length occurs approximately 5 DEG C. below the melting temperature for a given duplex. Incubation at temperatures below the optimum may allow mismatched sequences to hybridize and can therefor result in reduced specificity. When using RNA oligonucleotides with formamide (50% v/v) it is recommend to use a hybridization temperature of 68 DEG C. for detection of target RNA and of 50 DEG C. for detection of target DNA. Alternatively, a high SDS hybridization solution can be utilized (Church et al. 1984, Proc Natl Acad Sci USA 81:1991-1995). The specificity of hybridization can furthermore be ensured through the presence of a crosslinking moiety on the oligonucleotide (e.g. Huan et al. 2000, Biotechniques 28: 254-255; WO00/14281). Said crosslinking moiety enables covalent linking of the oligonucleotide with the target nucleotide sequence and hence allows stringent washing conditions. Such a crosslinking oligonucleotide can furthermore comprise another label suitable for detection/quantification of the oligonucleotide hybridized to the target.

RPKM (Reads Per Kilobase Million) is often used as measure for expression. FPKM (Fragments Per Kilobase Million) is very similar to RPKM; whereas RPKM was designed for single-end RNA-seq (every read corresponded to a single sequenced fragment), FPKM was designed for paired-end RNA-seq. With paired-end RNA-seq, two reads can correspond to a single fragment, or, if one read in the pair did not map, one read can correspond to a single fragment. The only difference between RPKM and FPKM is that FPKM takes into account that two reads can map to one fragment (and so it doesn't count this fragment twice). When using RNA-seq, reporting or results often is in RPKM (Reads Per Kilobase Million) or FPKM (Fragments Per Kilobase Million). Whatever metric used (another alternative for example is TPM (Transcripts Per Kilobase Million)), such metric is attempting to normalize for sequencing depth and gene length and provide a measure for quantifying transcript levels/gene expression/expression units.

Next to methodologies for determining gene expression by means of determining transcript levels (transcriptome analysis), it is also possible to quantify gene expression by means of proteomic analysis (proteome analysis or analysis of the proteome). Classical proteomic analysis methods include ELISA, western blotting, mass spectrometry, chromatographic separation, immunohistochemistry, cell sorting (based on cell surface marker(s)) etc. Although not necessarily required, it can be advantageous to rely on multiplexed cytometry methods that can be performed directly on, e.g., a section of a breast cancer tissue biopsy (Formalin-Fixed Paraffin-Embedded (FFPE), fresh frozen (FF), . . . ). Multiplexed cytometry methods, as well as some predictive cancer biomarkers identified using such methodology, have been reviewed by e.g. Fan et al. 2020 (Cancer Communications 40:135-153) and have emerged with the advent of more sophisticated imaging techniques (e.g. cyclic immunofluorescence, tyramide-based immunofluorescence, epitope-targeted mass spectrometry, RNA detection) and standardized quantification methodologies. Such multiplexed cytometry methods include multiplex immunocytochemistry (mICH), imaging mass spectrometry, multiplexed ion beam imaging, chipcytometry, nucleotide (DNA/RNA)-barcoding-based mICH, and digital spacing profiling. Another technique involving proteomic analysis is Cellular Indexing of Transcriptomes and Epitopes by Sequencing (CITE-seq), as used in the Examples herein.

### Standard or Control/Standard or Control Expression Level

Standards or controls for the expression level (at transcriptomic level or at proteomic level) of a biomarker gene as listed above (i.e. any of IGHG1, IGHG2, IGHG3, IGLC2, HMGB2, IGKC, MZB1, SEC11C, BATF, CXCL13, CXCR6, GZMB, PDCD1, TIGIT or TNFRSF18) can be defined in some alternative ways.

In one embodiment, such standard or control expression level refers to a pre-determined range of expression levels/standard values. Typically such ranges are defined after collecting a set of expression levels of a gene X (i.e., any one of genes IGHG1, IGHG2, IGHG3, IGLC2, HMGB2, IGKC, MZB1, SEC11C, BATF, CXCL13, CXCR6, GZMB, PDCD1, TIGIT or TNFRSF18) as determined in a suitable number of breast cancer patients. As outlined in the Examples herein, the expression of the biomarker genes as listed above (i.e., any one of genes IGHG1, IGHG2, IGHG3, IGLC2, HMGB2, IGKC, MZB1, SEC11C, BATF, CXCL13, CXCR6, GZMB, PDCD1, TIGIT or TNFRSF18) is higher in future responders to immunotherapy or immunogenic therapy versus/compared to in future non-responders to immunotherapy or immunogenic therapy. From these, suitable standard/control expression levels or expression level ranges can be determined.

The expression level of a gene X (i.e., any one of genes IGHG1, IGHG2, IGHG3, IGLC2, HMGB2, IGKC, MZB1, SEC11C, BATF, CXCL13, CXCR6, GZMB, PDCD1, TIGIT or TNFRSF18) as determined in any of the above methods for a breast cancer patient potentially eligible for treatment with an immunotherapy or immunogenic therapy (the test subject) can alternatively be compared with the expression level at a similar time-point of the same gene X in a breast cancer patient or set of breast cancer patients known as (subsequent) responder(s) (or non-responder(s)) to the immunotherapy or immunogenic therapy (the control subject(s)). If the expression level of the gene X in the test subject is (roughly/about) equal to the expression level in a responding control subject, or is higher than the expression level in a non-responding control subject, then the test subject is predicted to be a responder to the immunotherapy or immunogenic therapy. If the expression level of the gene X in the test subject is (roughly/about) equal to the expression level in a non-responding control subject, or is lower than the expression level in a non-responding control subject, then the test subject is predicted to be a non-responder to the immunotherapy or immunogenic therapy.

In particular, the expression level of a gene X is determined by normalization relative to expression of e.g. a housekeeping gene or set of housekeeping genes. Any diagnostic kit or device designed to operate according to any of the above-listed methods of the invention (see further) therefore includes the option/possibility to determine, assess, measure, quantify expression of one or more household genes in addition to the means to determine, assess, measure, quantify expression of one or more of the above-listed biomarker genes predictive for outcome of immunotherapy or immunogenic therapy in a subject having breast cancer (i.e., any one individual, or any one combination of genes IGHG1, IGHG2, IGHG3, IGLC2, HMGB2, IGKC, MZB1, SEC11C, BATF, CXCL13, CXCR6, GZMB, PDCD1, TIGIT or TNFRSF18), or predictive for (early) response or predictive for (early) T-cell response of a subject having breast cancer to immunotherapy or immunogenic therapy.

The higher expression of a biomarker genes as listed above in future responders to immunotherapy or immunogenic therapy versus/compared to in future non-responders to immunotherapy or immunogenic therapy can be higher with 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 100% or more; or with up to 10%, up to 20%, of up to 30%, of up to 40%, of up to 50%, of up to 60%, of up to 70%, of up to 80%, of up to 90%, or with up to 100% or more. In case of a 100% higher analyte strand number of an individual biomarker gene, the expression of that individual biomarker has doubled, or increased 2-fold. The higher analyte strand number of an individual biomarker can further be 1.1-fold higher, 1.2-fold higher, 1.3-fold higher, 1.4-fold higher, 1.5-fold higher, 1.6-fold higher, 1.7-fold higher, 1.8-fold higher, 1.9-fold higher, 2-fold higher, 2.1-fold higher, 2.2-fold higher, 2.3-fold higher, 2.4-fold higher, 2.5-fold higher, 2.6-fold higher, 2.7-fold higher, 2.8-fold higher, 2.9-fold higher, 3-fold higher, more than 3-fold higher, 3.5-fold higher, 4-fold higher, more than 4-fold higher, between 3-fold and 4-fold higher, 4.5-fold higher, 5-fold higher, more than 5-fold higher, between 2-fold and 5-fold higher, between 3-fold and 5-fold higher, between 3-fold and 5-fold higher, 6-fold higher, more than 6-fold higher, between 2-fold and 6-fold higher, between 3-fold and 6-fold higher, between 4-fold and 6-fold higher, 7-fold higher, more than 7-fold higher, 8-fold higher, lore than 8-fold higher, 9-fold higher, more than 9-fold higher, 10-fold higher, up to 10-fold higher, more than 10-fold higher, between 2-fold and 10-fold higher, between 3-fold and 10-fold higher, between 4-fold and 10-fold higher, between 5-fold and 10-fold higher, between 6-fold and 10-fold higher, between 7-fold and 10-fold higher, between 8-fold and 10-fold higher, substantially more than 10-fold higher, between 10-fold and 15-fold higher, up to 15-fold higher, between 10-fold and 20-fold higher, up to 20-fold higher, substantially more than 20-fold in higher crease such as up to 25-fold higher, up to 30-fold higher, up to 40-fold higher, or up to 50-fold higher.

### Further Aspects of the Invention

In one further aspect of the invention, any of the hereinabove described
methods of selecting, identifying, choosing, or collecting (or, contrary, of refusing or rejecting) a subject or subjects having breast cancer for treatment with an immunotherapy or with an immunogenic therapy; or
methods of selecting, identifying, choosing, or collecting a subject or subjects having breast cancer fit or proper for treatment with an immunotherapy or with an immunogenic therapy; or
methods of determining eligibility, susceptibility, qualification, suitability or acceptability of a subject having breast cancer for treatment with an immunotherapy or with an immunogenic therapy; or
methods of determining prior to start, at start, or after start of an immunotherapy or of an immunogenic therapy in a subject having breast cancer the outcome of the immunotherapy or of the immunogenic therapy; or
methods of determining prior to start, at start, or after start of an immunotherapy or of an immunogenic therapy in a subject having breast cancer the response or to the immunotherapy or to the immunogenic therapy; or
methods of determining prior to start, at start, or early after start of an immunotherapy or of an immunogenic therapy in a subject having breast cancer the early response to the immunotherapy or to the immunogenic therapy; or
methods of determining prior to start, at start, or early after start of an immunotherapy or of an immunogenic therapy in a subject having breast cancer the T-cell expansion response to the immunotherapy or to the immunogenic therapy; or
methods of determining prior to start, at start, or early after start of an immunotherapy or of an immunogenic therapy in a subject having breast cancer the early T-cell expansion response to the immunotherapy or to the immunogenic therapy; or
methods of monitoring the response of a subject having breast cancer to an immunotherapy or to an immunogenic therapy;
   can further comprise:
quantifying one or more of the Gini-index of T-cell receptor clonotype distribution, the relative frequency of exhausted/experienced T-cells, the relative frequency of exhausted/experienced CD4+ T-cells, the relative frequency of exhausted/experienced CD8+ T-cells, the T-cell receptor richness, or the relative frequency of the C7_CX3CR1 subtype of T-cell inhibitory macrophages;
   wherein high values within a pre-determined range of values of the Gini-index of T-cell receptor clonotype distribution, the relative frequency of exhausted/experienced T-cells, the relative frequency of exhausted/experienced CD4+ T-cells, and/or of the relative frequency of exhausted/experienced CD8+ T-cells are further indicative of a positive outcome of (and thus of selecting a subject having breast cancer, or of determining a subject having breast cancer to be eligible, for treatment with the immunotherapy or the immunogenic therapy); or, (early) response to, or (early) T-cell response to the immunotherapy or of the immunogenic therapy;
   and low values within a pre-determined range of values of the T-cell receptor richness, and/or of the relative frequency of the C7_CX3CR1 subtype of T-cell inhibitory macrophages are further indicative of a positive outcome of (and thus of selecting a subject having breast cancer, or of determining a subject having breast cancer to be eligible, for treatment with the immunotherapy or the immunogenic therapy); or, (early) response to, or (early) T-cell response to the immunotherapy or of the immunogenic therapy. More detailed information on these features is provided in Example 2.1.

In one embodiment, the quantification of the gene expression level in any of the methods according to the invention is determined by transcriptome analysis and/or is determined by proteome analysis. Means and methods of transcriptome and proteome analysis are outlined above. In a further embodiment, any expression level is determined in vitro in a sample obtained from the subject having breast cancer, or on a component isolated from that sample. In a particular embodiment, the transcriptome analysis is not an analysis on/at the single cell level and not an analysis on/at the cell subtype level (e.g. CD4+ T-cells, B-cells, . . . ). In a particular embodiment, the transcriptome analysis is an analysis on/of the bulk transcriptome. As outlined above, e.g. increased expression levels of a gene X determined at single cell level or at cell subtype level can become irrelevant/non-detectable/non-significant when determined in/for the bulk transcriptome.

In a further embodiment the breast cancer is early breast cancer.

In a further aspect of the invention, any of the hereinabove described methods of the invention can further be extended by including detecting the status of one or more further diagnostic markers or biomarkers selected from immune checkpoint gene expression, markers of tumor mutational burden, T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES), expression of retrotransposons, infiltration of immune cells in the tumor (B-cells/T-cells/macrophages, etc.) of a subject having breast cancer, wherein the status of the one or more further diagnostic markers or biomarkers is further indicative of the positive outcome of the immunotherapy or of the immunogenic therapy.

In one further aspect of the invention, any of the hereinabove described
methods of selecting, identifying, choosing, or collecting (or, contrary, of refusing or rejecting) a subject or subjects having breast cancer for treatment with an immunotherapy or with an immunogenic therapy; or
methods of selecting, identifying, choosing, or collecting a subject or subjects having breast cancer fit or proper for treatment with an immunotherapy or with an immunogenic therapy; or
methods of determining eligibility, susceptibility, qualification, suitability or acceptability of a subject having breast cancer for treatment with an immunotherapy or with an immunogenic therapy; or
methods of determining prior to start, at start, or after start of an immunotherapy or of an immunogenic therapy in a subject having breast cancer the outcome of the immunotherapy or of the immunogenic therapy; or
methods of determining prior to start, at start, or after start of an immunotherapy or of an immunogenic therapy in a subject having breast cancer the response or to the immunotherapy or to the immunogenic therapy; or
methods of determining prior to start, at start, or early after start of an immunotherapy or of an immunogenic therapy in a subject having breast cancer the early response to the immunotherapy or to the immunogenic therapy; or
methods of determining prior to start, at start, or early after start of an immunotherapy or of an immunogenic therapy in a subject having breast cancer the T-cell expansion response to the immunotherapy or to the immunogenic therapy; or
methods of determining prior to start, at start, or early after start of an immunotherapy or of an immunogenic therapy in a subject having breast cancer the early T-cell expansion response to the immunotherapy or to the immunogenic therapy; or
methods of monitoring the response of a subject having breast cancer to an immunotherapy or to an immunogenic therapy;
   can further comprise a step of administering a therapeutically effective amount of the immunotherapeutic or immunogenic agent to a selected subject or to a subject in which a positive outcome of, positive (early) response to, or (early) T-cell expansion response to the immunotherapeutic or immunogenic agent is indicated, predicted or determined.

In one embodiment, the immunotherapy or immunogenic therapy, or immunotherapeutic or immunogenic agent, in any of the hereinabove described methods of the invention is a therapy or agent comprising an immune checkpoint blocker. In a particular embodiment, the immune checkpoint blocker is a molecule (such as an antibody) blocking PD1.

In a further aspect of the invention, at least one data collection step or at least one analysis step in any of the hereinabove described methods of the invention is performed by a computer system or via a computer program product.

Therefore, in a further aspect, the invention relates to computer products comprising a computer readable medium storing instructions for operating a computer system to perform at least one data collection step or at least one analysis step in any of the hereinabove described methods of the invention.

Further aspects of the invention relate to using immunotherapy or immunogenic therapy in patients that are most likely to respond to the immunotherapy or immunogenic therapy. As outlined herein, such decision can be made based on expression levels of one or more of the biomarker genes identified herein. A further aspect of the invention thus relates to immunotherapeutic or immunogenic agents for use in (a method of) treating a subject having breast cancer, for use in (a method of) inhibiting breast cancer progression or relapse, or for use in (a method of) inhibiting breast cancer metastasis; or relates to use of an immunotherapeutic or immunogenic agents for (use in formulating a medicament for) treating of breast cancer, for (use in formulating or manufacturing a medicament for) inhibiting breast cancer progression or relapse, or for (use in formulating or manufacturing a medicament for) inhibiting breast cancer metastasis, comprising:
quantifying in a sample obtained from a subject having breast cancer prior to start, at start, or early after start of therapy comprising/of treatment with/or of administering or administration of the immunotherapeutic or immunogenic agent the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18 (as extensively defined hereinabove);
selecting a subject having breast cancer for treatment or for continued treatment with the immunotherapeutic or immunogenic agent, or for therapy or continued therapy comprising the immunotherapeutic or immunogenic agent, when the expression level quantified for the selected gene is within a pre-determined range of expression levels/standard values of the gene wherein the pre-determined range of expression levels is indicative of/associated with positive outcome of treatment with the immunotherapeutic or immunogenic agent. In particular the treatment or continued treatment with the immunotherapeutic or immunogenic agent is leading to/resulting in treatment of the subject having breast cancer, inhibition of breast cancer progression or relapse (in the subject having breast cancer), or inhibiting breast cancer metastasis (in the subject having breast cancer).

In one embodiment to this aspect, a step of administering a therapeutically effective amount of the immunotherapeutic or immunogenic agent to a selected subject can be added.

Alternatively, the invention relates to immunotherapeutic or immunogenic agents for use in (a method of) treating a subject having breast cancer, for use in (a method of) inhibiting breast cancer progression or relapse, or for use in (a method of) inhibiting breast cancer metastasis; or relates to use of an immunotherapeutic or immunogenic agents for (use in formulating a medicament for) treating of breast cancer, for (use in formulating or manufacturing a medicament for) inhibiting breast cancer progression or relapse, or for (use in formulating or manufacturing a medicament for) inhibiting breast cancer metastasis, comprising:
quantifying in a sample obtained from a subject having breast cancer prior to start, at start, or early after start of therapy comprising/of treatment with/or of administering or administration of the immunotherapeutic or immunogenic agent the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18 (as extensively defined hereinabove);
administering or continued administering of the immunotherapeutic or immunogenic agent to the subject having breast cancer, or treatment or continued treatment of the subject having breast cancer with the immunotherapeutic or immunogenic agent, or subjecting the subject having breast cancer to therapy or continued therapy comprising the immunotherapeutic or immunogenic agent, when the expression level quantified for the selected gene is within a pre-determined range of expression levels/standard values of the gene wherein the pre-determined range of expression levels is indicative of/associated with positive outcome of the immunotherapy or of the immunogenic therapy. In particular the (continued) treatment with/administering of/therapy comprising the immunotherapeutic or immunogenic agent is leading to treatment of the subject having breast cancer, in inhibition of breast cancer progression or relapse (in the subject having breast cancer), or inhibiting breast cancer metastasis (in the subject having breast cancer).

Further alternatively, the invention relates to immunotherapeutic or immunogenic agents for use in (a method of) treating a subject having breast cancer, for use in (a method of) inhibiting breast cancer progression or relapse, or for use in (a method of) inhibiting breast cancer metastasis; or relates to use of an immunotherapeutic or immunogenic agents for (use in formulating a medicament for) treating of breast cancer, for (use in formulating or manufacturing a medicament for) inhibiting breast cancer progression or relapse, or for (use in formulating or manufacturing a medicament for) inhibiting breast cancer metastasis, comprising:
quantifying in a sample obtained from a subject having breast cancer prior to start, at start, or early after start of therapy comprising/of treatment with/or of administering or administration of the immunotherapeutic or immunogenic agent the expression level of at least one gene selected from the genes IGHG1, IGHG2, IGHG3, IGLC2, HMGB2, IGKC, MZB1, SEC11C, BATF, CXCL13, CXCR6, GZMB, PDCD1, TIGIT or TNFRSF18 (as extensively defined hereinabove);
treating the subject having breast cancer, inhibiting breast cancer progression or relapse in the subject having breast cancer, or inhibiting breast cancer metastasis in the subject having breast cancer by administering or continued administering of the immunotherapeutic or immunogenic agent to the subject having breast cancer, or treatment or continued treatment of the subject having breast cancer with the immunotherapeutic or immunogenic agent, or subjecting the subject having breast cancer to therapy or continued therapy comprising the immunotherapeutic or immunogenic agent, when the expression level quantified for the selected gene is within a pre-determined range of expression levels/standard values of the gene wherein the pre-determined range of expression levels is indicative of/associated with positive outcome of the immunotherapy or of the immunogenic therapy.

In particular to the latter alternative aspects of the invention, the positive outcome of the immunotherapy or of the immunogenic therapy is, or is adding to, treatment of the subject having breast cancer, inhibition of breast cancer progression or relapse (in the subject having breast cancer), or is inhibition of breast cancer metastasis (in the subject having breast cancer).

In a further embodiment to the latter alternative aspects of the invention, the immunotherapy or immunogenic therapy, immunotherapeutic or immunogenic agent, is a therapy or agent comprising an immune checkpoint blocker. In a particular embodiment, the immune checkpoint blocker is a molecule (such as an antibody) blocking PD1.

In yet a further aspect, the invention relates to use of a panel of biomarker genes of the invention in any of the above described methods of the invention, wherein the panel is comprising 1 to 15 of the biomarker genes, i.e. 1 to 15 genes selected from IGHG1, IGHG2, IGHG3, IGLC2, IGKC, HMGB2, MZB1, SEC11C, BATF, CXCL13, CXCR6, GZMB, PDCD1, TIGIT and TNFRSF18 (combinations thereof having been elaborately described hereinabove).

In a further aspect, the invention is relating to kits, such as diagnostic kits or companion diagnostic kits, such as for use in any of the above described methods of the invention, wherein such kits are comprising the tools to detect the expression level of at least one biomarker according to the invention, such as 1 to 15 of the biomarker genes, i.e. 1 to 15 genes selected from IGHG1, IGHG2, IGHG3, IGLC2, IGKC, HMGB2, MZB1, SEC11C, BATF, CXCL13, CXCR6, GZMB, PDCD1, TIGIT and TNFRSF18 (combinations thereof having been elaborately described hereinabove).

In one embodiment, such kits/diagnostic kits/companion diagnostic kits can further include the tools for detecting/determining/assessing/assaying/measuring the status of one or more further diagnostic markers or biomarkers selected from T-cell receptor clonotype distribution, frequency of exhausted/experienced T-cells, frequency of exhausted/experienced CD4+θT-cells, frequency of exhausted/experienced CD8+ T-cells, T-cell receptor richness, frequency of the C7_CX3CR1 subtype of T-cell inhibitory macrophages; or selected from immune checkpoint gene expression, markers of tumor mutational burden, T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES)—these are detailed hereinafter.

In one specific embodiment, such kits/diagnostic kits/companion diagnostic kits are including the tools for detecting the status of, in total, at most 1000 markers, at most 950 markers, at most 900 markers, at most 850 markers, at most 800 markers, at most 750 markers, at most 700 markers, at most 650 markers, at most 600 markers, at most 550 markers, at most 500 markers, at most 450 markers, at most 400 markers, at most 350 markers, at most 300 markers, at most 250 markers, or at most 225, 200, 175, 150, 125, 111, 110, 105, 100, 95, 90, 85, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20,19, 18, 17, 16, 15, 14, 13, 12, 11, 10, 9, 8, 7, 6, 5, 4, 3, 2 markers, or at most 1 marker; in any case including at least one selected biomarker according to the invention as identified herein, i.e. at least on biomarker selected from IGHG1, IGHG2, IGHG3, IGLC2, IGKC, HMGB2, MZB1, SEC11C, BATF, CXCL13, CXCR6, GZMB, PDCD1, TIGIT and TNFRSF18. Alternatively, such kits are including the tools for detecting the status of 1 to 10 markers, of 1 to 20 markers, of 1 to 50 markers, of 1 to 30 markers, of 1 to 50 markers, of 1 to 60 markers, of 1 to 70 markers, of 1 to 80 markers, of 1 to 90 markers, of 1 to 100 markers, of 1 to 150 markers, of 1 to 200 markers, of 1 to 300 markers, of 1 to 400 markers, of 1 to 500 markers, of 1 to 600 markers, of 1 to 700 markers, of 1 to 800 markers, of 1 to 900 markers, or of 1 to 1000 markers; in any case including at least one selected biomarker according to the invention as identified herein, i.e. at least on biomarker selected from IGHG1, IGHG2, IGHG3, IGLC2, IGKC, HMGB2, MZB1, SEC11C, BATF, CXCL13, CXCR6, GZMB, PDCD1, TIGIT and TNFRSF18.

In particular, the tools of a kit/diagnostic kit/companion diagnostic kit of the invention comprise, besides optionally e.g. reagents, enzymes, reaction vessels and kit inserts, oligonucleotides capable of detecting the status of an envisaged biomarker. In particular, the oligonucleotides comprise a sequence specifically hybridizing to said biomarker or in the immediate vicinity of said biomarker. In particular, the oligonucleotide is comprising least one modified or non-naturally occurring nucleotide (as described hereinabove). Further in particular, the oligonucleotide may be part of a primer and probe set, of which set at least one primer or probe is comprising a sequence specifically hybridizing to the envisaged biomarker or in the immediate vicinity of said biomarker. Such kits/diagnostic kits/companion diagnostic kits can alternatively comprise a multi-membered set of oligonucleotides, wherein each member of the set comprises at least one modified or non-naturally occurring nucleotide and a sequence specifically hybridizing to one of the biomarkers or in the immediate vicinity of said biomarker. Such kits/diagnostic kits/companion diagnostic kits can alternatively comprise a plurality of separate primer and probe sets, wherein each set is comprising a primer or probe comprising of which at least one of the primer or probe is comprising a modified or non-naturally occurring nucleotide, and wherein each set comprises a primer or probe of which at least one of the primer or probe is comprising a sequence specifically hybridizing to one of the biomarkers or in the immediate vicinity of said biomarker. A non-naturally occurring nucleotide may be a nucleotide that is chemically different from a nucleotide present in a living cell (such as a labelled nucleotide), or may be a chemically naturally occurring nucleotide but which is mutated relative to the natural target nucleic acid on which the oligonucleotide is specifically hybridizing.

In a further aspect, the invention relates to:
1) methods of selecting, identifying, choosing, or collecting (or, contrary, of refusing or rejecting) a subject or subjects having breast cancer for treatment with an immunotherapy or with an immunogenic therapy, comprising one or more steps of:
   quantifying in a sample obtained from the subject prior to start, at start, or early after start of the immunotherapy or of the immunogenic therapy one or more of the following features: the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages;
   selecting, identifying, choosing, collecting (or, contrary, refusing or rejecting) a subject having breast cancer for treatment with the immunotherapy or with the immunogenic therapy, when the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages are/is within a pre-determined value range indicative of/associated with positive outcome of the immunotherapy or of the immunogenic therapy; or
2) methods of selecting, identifying, choosing, or collecting a subject or subjects having breast cancer fit or proper for treatment with an immunotherapy or with an immunogenic therapy, comprising one or more steps of:
   quantifying in a sample obtained from the subject prior to start, at start, or early after start of the immunotherapy or of the immunogenic therapy one or more of the following features: the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages;
   selecting, identifying, choosing, or collecting a subject having breast cancer for treatment with the immunotherapy or with the immunogenic therapy, when the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages are/is within a pre-determined value range indicative of/associated with positive outcome of the immunotherapy or of the immunogenic therapy; or
3) methods of determining eligibility, susceptibility, qualification, suitability or acceptability of a subject having breast cancer for treatment with an immunotherapy or with an immunogenic therapy, comprising one or more steps of:
   quantifying in a sample obtained from the subject prior to start, at start, or early after start of the immunotherapy or of the immunogenic therapy one or more of the following features: the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages;
   determining a subject having breast cancer to be eligible, susceptible, qualified, suited or acceptable for treatment with the immunotherapy or with the immunogenic therapy, when the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages are/is within a pre-determined value range indicative of/associated with positive outcome of the immunotherapy or of the immunogenic therapy; or
4) methods of determining prior to start, at start, or after start of an immunotherapy or of an immunogenic therapy in a subject having breast cancer the outcome of the immunotherapy or of the immunogenic therapy, comprising one or more steps of:
   quantifying in a sample obtained from the subject prior to start, at start, or early after start of the immunotherapy or of the immunogenic therapy one or more of the following features: the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages;
   determining (prior to start, at start, or after start of an immunotherapy or of an immunogenic therapy in a subject having breast cancer) the outcome of the immunotherapy or of the immunogenic therapy to be positive, when the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages are/is within a pre-determined value range indicative of/associated with positive outcome of the immunotherapy or of the immunogenic therapy; or
5) methods of determining prior to start, at start, or after start of an immunotherapy or of an immunogenic therapy in a subject having breast cancer the response to the immunotherapy or to the immunogenic therapy, comprising one or more steps of:
   quantifying in a sample obtained from the subject prior to start, at start, or early after start of the immunotherapy or of the immunogenic therapy one or more of the following features: the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages;
   determining (prior to start, at start, or after start of an immunotherapy or of an immunogenic therapy in a subject having breast cancer) the response to the immunotherapy or of the immunogenic therapy to be positive, when the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages are/is within a pre-determined value range indicative of/associated with a positive response to the immunotherapy or to the immunogenic therapy; or
6) methods of determining prior to start, at start, or early after start of an immunotherapy or of an immunogenic therapy in a subject having breast cancer the early response to the immunotherapy or to the immunogenic therapy, comprising one or more steps of:
   quantifying in a sample obtained from the subject prior to start, at start, or early after start of the immunotherapy or of the immunogenic therapy one or more of the following features: the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages;
   determining (prior to start, at start, or after start of an immunotherapy or of an immunogenic therapy in a subject having breast cancer) the early response to the immunotherapy or of the immunogenic therapy to be positive, when the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages are/is within a pre-determined value range indicative of/associated with an early positive response/positive early response to the immunotherapy or to the immunogenic therapy; or
7) methods of determining prior to start, at start, or early after start of an immunotherapy or of an immunogenic therapy in a subject having breast cancer the T-cell expansion response to the immunotherapy or to the immunogenic therapy, comprising one or more steps of:
   quantifying in a sample obtained from the subject prior to start, at start, or early after start of the immunotherapy or of the immunogenic therapy one or more of the following features: the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages;
   determining (prior to start, at start, or after start of an immunotherapy or of an immunogenic therapy in a subject having breast cancer) the T-cell expansion response to the immunotherapy or of the immunogenic therapy to be positive, when the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages are/is within a pre-determined value range indicative of/associated with a T-cell response to the immunotherapy or to the immunogenic therapy; or
8) methods of determining prior to start, at start, or early after start of an immunotherapy or of an immunogenic therapy in a subject having breast cancer the early T-cell expansion response to the immunotherapy or to the immunogenic therapy, comprising one or more steps of:
   quantifying in a sample obtained from the subject prior to start, at start, or early after start of the immunotherapy or of the immunogenic therapy one or more of the following features: the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages;
   determining (prior to start, at start, or after start of an immunotherapy or of an immunogenic therapy in a subject having breast cancer) the T-cell expansion response to the immunotherapy or of the immunogenic therapy to be positive, when the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages are/is within a pre-determined value range indicative of/associated with an early T-cell expansion response to the immunotherapy or to the immunogenic therapy; or
9) methods of monitoring the response of a subject having breast cancer to an immunotherapy or to an immunogenic therapy, comprising one or more steps of:
   quantifying in a sample obtained from the subject prior to start, at start, and/or (early) after start of the immunotherapy or of the immunogenic therapy one or more of the following features: the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages;
   determining the outcome of, the response to, the early response to, the T-cell response to, or the early T-cell response to the immunotherapy or of the immunogenic therapy to be positive, when the Gini-index of T-cell receptor clonotype distribution, the relative frequency of exhausted/experienced T-cells, the relative frequency of exhausted/experienced CD4+ T-cells, the relative frequency of exhausted/experienced T-cells, the T-cell receptor richness, and/or the relative frequency of the C7_CX3CR1 subtype of T-cell inhibitory macrophages are/is within a pre-determined value range indicative of/associated with a positive outcome of, positive response to, positive early response to the immunotherapy or to the immunogenic therapy; or is indicative of/associated with T-cell expansion or early T-cell expansion in response to the immunotherapy or to the immunogenic therapy.

In particular, high values within a pre-determined range of (standard) values of the Gini-index of T-cell receptor clonotype distribution, the relative frequency of exhausted/experienced T-cells, the relative frequency of exhausted/experienced CD4+ T-cells, and/or of the relative frequency of exhausted/experienced CD8+ T-cells are further indicative of a positive outcome of (and thus of selecting a subject having breast cancer, or of determining a subject having breast cancer to be eligible, for treatment with the immunotherapy or the immunogenic therapy); or, (early) response to, or (early) T-cell response to the immunotherapy or of the immunogenic therapy;

and low values within a pre-determined range of (standard) values of the T-cell receptor richness, and/or of the relative frequency of the C7_CX3CR1 subtype of T-cell inhibitory macrophages are further indicative of a positive outcome of (and thus of selecting a subject having breast cancer, or of determining a subject having breast cancer to be eligible, for treatment with the immunotherapy or the immunogenic therapy); or, (early) response to, or (early) T-cell response to the immunotherapy or of the immunogenic therapy.

More detailed information on these features is provided in Example 2.1.

The indicated (standard) value ranges can be determined in a similar way as explained hereinabove for determining standard value ranges of gene/biomarker expression level.

Combination with Further Diagnostic Markers

Any of the above described methods of the invention, i.e.:
methods of selecting, identifying, choosing, or collecting (or, contrary, of refusing or rejecting) a subject or subjects having breast cancer for treatment with an immunotherapy or with an immunogenic therapy; or
methods of selecting, identifying, choosing, or collecting a subject or subjects having breast cancer fit or proper for treatment with an immunotherapy or with an immunogenic therapy; or
methods of determining eligibility, susceptibility, qualification, suitability or acceptability of a subject having breast cancer for treatment with an immunotherapy or with an immunogenic therapy; or
methods of determining prior to start, at start, or after start of an immunotherapy or of an immunogenic therapy in a subject having breast cancer the outcome of the immunotherapy or of the immunogenic therapy; or
methods of determining prior to start, at start, or after start of an immunotherapy or of an immunogenic therapy in a subject having breast cancer the response or to the immunotherapy or to the immunogenic therapy; or
methods of determining prior to start, at start, or early after start of an immunotherapy or of an immunogenic therapy in a subject having breast cancer the early response to the immunotherapy or to the immunogenic therapy; or
methods of determining prior to start, at start, or early after start of an immunotherapy or of an immunogenic therapy in a subject having breast cancer the T-cell expansion response to the immunotherapy or to the immunogenic therapy; or
methods of determining prior to start, at start, or early after start of an immunotherapy or of an immunogenic therapy in a subject having breast cancer the early T-cell expansion response to the immunotherapy or to the immunogenic therapy; or
methods of monitoring the response of a subject having breast cancer to an immunotherapy or to an immunogenic therapy;
   may be supplemented with one or more steps of determining/assessing/assaying/detecting/measuring the status of further diagnostic markers or biomarkers. Such further diagnostic markers or biomarkers non-exhaustively include markers such as nucleotide substitution number, number of indels (insertions or deletions), immune cytolytic activity, T cell-inflamed gene expression signature, IFN-γ related gene expression signature, type I and type II interferon-related gene expression, immunopredictive score, and expression of immune checkpoint genes.

As indicated by Cristescu et al. 2018 (Science 362:197) for instance, expression of ligand 1 of the immune checkpoint gene PD-1 (PD-L1) is a clinically validated biomarker for response to the PD-1-inhibitor pembrolizumab, just as is high microsatellite instability (the latter regardless of tumor type). The same authors elaborated on the applicability of tumor mutational burden (TMB) and the T cell-inflamed gene expression profile (GEP) as biomarkers for predicting response to immune checkpoint inhibition therapy, and concluded that both sets independently predict response, but nevertheless provide complementary information enabling subgrouping of tumours which may further guide precision cancer immunotherapy. This study highlights the importance of tumor analysis or of tumor profiling based on different sets of marker types (such as related to the tumor cell status, status of immune compartment of the tumor, and status of the tumoral microenvironment), even if the individual sets themselves already have some independent predictive value. Thus, another biomarker that can be combined with the herein described biomarkers is immune cytolytic activity, such as developed by Rooney et al. 2015 (Cell 160:48-61) and which relies on determination of transcript levels of two cytolytic effectors, granzyme A (GZMA) and perforin (PRF1). Other possible markers complementary or supplementary to the herein described biomarkers are interferon-related gene expression signatures. Such genes are e.g. genes known to be induced by type I interferons, type II interferons (see, e.g., Hall et al. 2012, Proc Natl Acad Sci USA 109:17609-17614; and see Example 12) or by interferon gamma (IFN-γ). A limited IFN-γ signature contains the genes IFNG, STAT1, CCR5, CXCL9, CXCL10, CXCL11, IDO1, PRF1, GZMA, and MHCII HLA-DRA (note that the cytolytic markers PRF1 and GZMA are included herein). An extended IFN-γ signature contains further cytolytic markers, chemokine and chemokine receptors, T cell markers, markers of NK cell activity, antigen presentation genes and immunomodulatory factors: granzyme B (GZMB), granzyme K (GZBK), CXCR6, CCL5, CD3D, CD3E, CD2, CXCL13, CXCL10, IL2RG, NKG7, HLA-E, CIITA, LAG3, IDO1, SLAMF6, TAGAP, STAT1 (Ayers et al. 2017, J Clin Invest 127:2930-2940).

High microsatellite instability (MSI) has been recognized by the FDA as a relevant biomarker for predicting response to anti-PD-1 therapy (see above). Historical markers of MSI include the markers of the revised Bethesda panel (Boland et al., 1998; Dietmaier et al., 1997), including the markers BAT25, BAT26, D5S346, D17S250, D2S123, BAT40, D17S787, D18S58, D18S69, and TGFP-RII. Based on whole genome sequencing analysis, many more MSI marker were identified in WO 2013/153130, including indel mutations in homopolymer sequences occurring in 5′UTR, 3′UTR, and exon regions of several genes (see Tables 1 and 2 of WO 2013/153130). Determination of MSI status can be combined with the herein described quantification of expression of the biomarkers of the invention. MSI can be considered in part to contribute to the overall tumor mutation load or burden (TMB). Tumor mutational burden can also be determined by sequencing genes known to be subject to mutation in tumours. Table 5 of US 2019/0018926 for instance provides an extensive list of “mutational burden genes”. In general, US 2019/0018926 relates to methodologies for generating an immune-oncology profile of a given tumor sample. Detecting the tumor mutation burden and/or generating an immune-oncology profile of tumor (such as by the methods of US 2019/0018926) can be combined with the herein described quantification of expression of the biomarkers of the invention.

Increased expression of hypoxia marker genes is a source of further possible markers complementary or supplementary to the herein described quantification of expression of the biomarkers of the invention. Such hypoxia marker genes can be one or more of the genes BNIP3, EGLN3, CA9, or ALDOA as used herein; or can be one or more of the genes described by Sorensen et al. 2015 (Radiother Oncol 116:346-351): ADM (adrenomedullin), ALDOA (Aldolase, Fructose-Bisphosphate A), ANKRD37 (Ankyrin Repeat Domain 37), BNIP3 (BCL2 Interacting Protein 3), BNIP3L (BCL2 Interacting Protein 3-Like), EGLN3 (Egl-9 Family Hypoxia Inducible Factor 3), FAM162A (Family With Sequence Similarity 162 Member A), KCTD11 (Potassium Channel Tetramerization Domain Containing 11), LOX (Lysyl Oxidase), NDRG1 (N-Myc Downstream Regulated 1), P4HA1 (Prolyl 4-Hydroxylase Subunit Alpha 1), P4HA2 (Prolyl 4-Hydroxylase Subunit Alpha 2), PDK1 (Pyruvate Dehydrogenase Kinase 1), PFKB3 (6-Phosphofructo-2-Kinase/Fructose-2,6-Biphosphatase 3), SLC2A1 (Solute Carrier Family 2 Member 1, also known as GLUT1). These authors also mention OPN (osteopontin) and LDH (lactate dehydrogenase) as hypoxia markers. An alternative way to determine the hypoxia status of a tumor, and thus alternative markers therefore, is by determining the hypermethylation status of one or more promoters of tumor suppressor genes (TSGs) HIC1, KDM6A, NF2, KDM5C, IGFBP2, ARNT2, PTEN, MGMT, ATM, MLH1, BRCA1, SEMA3B, TIMP3, THBD, and CLDN3. Increased hypermethylation in TSG promoters is indicative of a hypoxic tumor (see WO 2016/142295A1).

Other markers providing information about a tumor or its environment that can complement or supplement the herein described quantification of expression of the biomarkers of the invention include the detection of the expression of immunomodulatory genes. Examples of immune modulatory molecules include, but are not limited to, one or more of 2B4 (CD244), A2aR, B7H3 (CD276), B7H4 (VTCN1), B7H6, B7RP1, BTLA (CD272), butyrophilins, CD103, CD122, CD137 (4-1BB), CD137L, CD160, CD2, CD200R, CD226, CD26, CD27, CD28, CD30, CD39, CD40, CD48, CD70, CD73, CD80 (B7.1), CD86 (B7.2), CEACAM1, CGEN-15049, CTLA-4, DR3, GAL9, GITR, GITRL, HVEM, ICOS, ICOSL (B7H2), IDOL, IDO2, ILT-2 (LILRB1), ILT-4 (LILRB2), KIR, KLRG1, LAG3, LAIR1 (CD305), LIGHT (TNFSF14), MARCO, NKG2A, NKG2D, OX-40, OX-40L, PD-1, PDL-1 (B7-H1, CD 274), PDL-2 (B7-DC, CD 273), PS, SIRPalpha, CD47, SLAM, TGFR, TIGIT, TIM1, TIM3 (HAVCR2), TIM4, or VISTA. In one specific setup, the expression of immune checkpoint genes is used to build the immune-predictive score (IMPRES; Auslander et al. 2018, Nature Med 24:1545-1549) relying on immune checkpoint inhibitors (ADORA2A, BTLA, VISTA, CD200, CD200R1, PDL-1, CD276, CD80, CD86, CEACAM1, CTLA4, GAL3, TIM-3, IDO1, KIR3DL1, LAG3, LAIR1, PD-1, PD-1LG2, PVR, PVRL2, TIGIT, VTCN1) and immune checkpoint activators (CD266, CD27, CD28, CD40, CD40L, CD70LG, DR3, HAVCR1, ICOS, ICOSL, IL2RB, NAIL, SLAM, TIM2, HVEM, TNFRSF18, TNFRSF4, TNFRSF9, TNFSF14, TNFSF18, OX40L, CD137L) which are paired (see Auslander et al. 2018 for details). Again, quantification of expression of the biomarkers of the invention can be combined with determining such immune-predictive score. Quantification of expression of the biomarkers of the invention can further be combined with detecting expression of one or more immune checkpoint inhibitors and/or with detecting expression of one or more immune checkpoint activators. Immune checkpoint inhibitor genes include ADORA2A, BTLA, VISTA, CD200, CD200R1, PDL-1, CD276, CD80, CD86, CEACAM1, CTLA4, GAL3, TIM-3, IDO1, KIR3DL1, LAG3, LAIR1, PD-1, PD-1LG2, PVR, PVRL2, TIGIT, and VTCN1. Immune checkpoint activator genes include CD266, CD27, CD28, CD40, CD40L, CD70LG, DR3, HAVCR1, ICOS, ICOSL, IL2RB, NAIL, SLAM, TIM2, HVEM, TNFRSF18, TNFRSF4, TNFRSF9, TNFSF14, TNFSF18, OX40L, and CD137L. Further markers providing information about a tumor or its environment that can complement or supplement the herein described quantification of expression of the biomarkers of the invention include detecting the status of an innate anti-PD-1 resistance gene expression signature (IPRES) such as described by Hugo et al. 2016 (Cell 165:35-44). The IPRES scoring relying on enrichment of an innate anti-PD-1 resistance gene expression signature including genes involved in mesenchymal transition, angiogenesis, hypoxia, and wound healing (see Hugo et al. 2016, Cell 165:35-44 for details).

Detecting the tumor immune cell composition in conjunction with quantification of expression of the biomarkers of the invention may also provide additional information about the tumor status. Examples of immune cells to be detected by methods described herein include, but are not limited to, CD4+ memory T-cells, CD4+ naive T-cells, CD4+ T-cells, central memory T (Tcm) cells, effector memory T (Tem) cells, CD4+ Tcm, CD4+ Tem, CD8+ T-cells, CD8+ naive T-cells, CD8+)Tcm, CD8+ Tem, regulatory T cells (Tregs), T helper (Th) 1 cells, Th2 cells, gamma delta T (Tgd) cells, natural killer (NK) cells, natural killer T (NKT) cells, B-cells, naive B-cells, memory B-cells, class-switched memory B-cells, pro B-cells, and plasma cells. In some instances, the sequencing data is used to determine expression of non-immune cells including, but not limited to, stromal cells, stem cells, or tumor cells. As indicator of the presence and/or amount of CD4+ T-cells, the expression of one or more of the following genes can be determined (US 2019/0018926, Table 1A): ALS2CL, ANKRD55, ZNF483, TRAV13-1, ST6GALNAC1, SEMA3A, TRBV5-4, DNAH8, IL2RA, TRBV11-2, TRAV8-2, KRT72, EPPK1, FAM153B, TRAV12-2, TRAV8-6, TRBV6-5, TRAV10, IGKV5-2, IGLV6-57, TRAV12-1, CTLA4, TSHZ2, FOXP3, IGHV4-28, TRAV2, SORCS3, TRAV5, MDS2, NTN4, IGLV10-54, DACT1, TRBV5-5, THEM5, HPCAL4, and/or CD4. As indicator of the presence and/or amount of CD8+ T-cells, the expression of one or more of the following genes can be determined (US 2019/0018926, Table 1B): FLT4, TRBV4-2, TRBV6-4, SPRY2, S100B, TNIP3, CD248, ROBO1, CD8B, TRBV2, CYP4F22, PZP, LAG3, KLRC4-KLRK1, CRTAM, SHANK1, ANAPC1P1, NRCAM, JAKMIP1, KLRC2, KLRC3, CD8A, TRAV4, FBLN2. As indicator of the presence and/or amount of monocytes, the expression of one or more of the following genes can be determined (US 2019/0018926, Table 1C): DES, HLX, FPR3, FCGR1B, LOXHD1, EPHB2, LPL, LIPN, AQP9, MILR1, RETN, GPNMB, CYP2S1, PDK4, LILRA6, SEPT10, PLA2G4A, FOLR2, FOLR3, C1QB, SLC6A12, SLC22A16, DOCK1, NRG1, RXFP2, RIN2, ARHGEF10L, LPAR1, CES1, FPR2. As indicator of the presence and/or amount of natural killer (NK) cells, the expression of one or more of the following genes can be determined (US 2019/0018926, Table 1D): IGFBP7, LDB2, GUCY1A3, KLRF1, DTHD1, AKR1C3, FASLG, KLRC1, XCL1, DAB2, FAT4, CD160, BNC2, CXCR1, SIGLEC171P, SH2D1B, DGKK, ZMAT4, LGALS9B, NMUR1, LGALS9C, MLC1, LIM2, NCR1, CCNJL, PCDH1. As indicator of the presence and/or amount of B-cells, the expression of one or more of the following genes can be determined (US 2019/0018926, Table 1E): UGT8, IGKV1OR2-108, IGHE, SCN3A, IGLV2-8, IGKV1D-16, MYO5B, ENAM, RP11-148021.2, IGLC7, IGHV1-2, IGKJ5, SOX5, TNFRSF13B, IGKV2D-29, IGKV1-17, IGLV2-18, IGHV2-70, CHL1, IGKV3D-20, IGLV8-61, IGKV6-21.

Based on the above, any of the hereinabove described methods of the invention, may be supplemented with one or more steps of determining/assessing/assaying/detecting/measuring the status of one or more further diagnostic markers or biomarkers. Such further diagnostic markers include immune checkpoint gene expression, markers of tumor mutational burden (such as substitutions, indels, microsatellite instability (MSI), providing substitution markers, indel markers and MSI-markers, respectively), T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES). As is clear from the above, detection of some of the individual additional biomarkers can be shared by different biomarker signatures.

### Tumor, Cancer, Neoplasm

The terms tumor and cancer are sometimes used interchangeably but can be distinguished from each other. A tumor refers to “a mass” which can be benign (more or less harmless) or malignant (cancerous). A cancer is a threatening type of tumor. A tumor is sometimes referred to as a neoplasm: an abnormal cell growth, usually faster compared to growth of normal cells. Benign tumors or neoplasms are non-malignant/non-cancerous, are usually localized and usually do not spread/metastasize to other locations. Because of their size, they can affect neighboring organs and may therefore need removal and/or treatment. A cancer, malignant tumor or malignant neoplasm is cancerous in nature, can metastasize, and sometimes re-occurs at the site from which it was removed (relapse). The initial site where a cancer starts to develop gives rise to the primary cancer. When cancer cells break away from the primary cancer (“seed”), they can move (via blood or lymph fluid) to another site even remote from the initial site. If the other site allows settlement and growth of these moving cancer cells, a new cancer, called secondary cancer, can emerge (“soil”). The process leading to secondary cancer is also termed metastasis, and secondary cancers are also termed metastases. For instance, liver cancer can arise as primary cancer, but can also be a secondary cancer originating from a primary breast cancer, bowel cancer or lung cancer; some types of cancer show an organ-specific pattern of metastasis. Most cancer deaths are in fact caused by metastases, rather than by primary tumors (Chambers et al. 2002, Nature Rev Cancer 2:563-572).

### Treatment/Therapeutically Effective Amount

“Treatment”/“treating” refers to any rate of reduction, delaying or retardation of the progress of the disease or disorder, or a single symptom thereof, compared to the progress or expected progress of the disease or disorder, or singe symptom thereof, when left untreated. This implies that a therapeutic modality on its own may not result in a complete or partial response (or may even not result in any response), but may, in particular when combined with other therapeutic modalities, contribute to a complete or partial response (e.g. by rendering the disease or disorder more sensitive to therapy). More desirable, the treatment results in no/zero progress of the disease or disorder, or singe symptom thereof (i.e. “inhibition” or “inhibition of progression”), or even in any rate of regression of the already developed disease or disorder, or singe symptom thereof. “Suppression/suppressing” can in this context be used as alternative for “treatment/treating”. Treatment/treating also refers to achieving a significant amelioration of one or more clinical symptoms associated with a disease or disorder, or of any single symptom thereof. Depending on the situation, the significant amelioration may be scored quantitatively or qualitatively. Qualitative criteria may e.g. by patient well-being. In the case of quantitative evaluation, the significant amelioration is typically a 10% or more, a 20% or more, a 25% or more, a 30% or more, a 40% or more, a 50% or more, a 60% or more, a 70% or more, a 75% or more, a 80% or more, a 95% or more, or a 100% improvement over the situation prior to treatment. The time-frame over which the improvement is evaluated will depend on the type of criteria/disease observed and can be determined by the person skilled in the art.

A “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, e.g., be measured by assessing the duration of survival (e.g. overall survival), time to disease progression (TTP), response rates (e.g., complete response and partial response, stable disease), length of progression-free survival, duration of response, and/or quality of life. The term “effective amount” refers to the dosing regimen of the agent (e.g. antagonist as described herein) or composition comprising the agent (e.g. medicament or pharmaceutical composition). The effective amount will generally depend on and/or will need adjustment to the mode of contacting or administration. The effective amount of the agent or composition comprising the agent is the amount required to obtain the desired clinical outcome or therapeutic effect without causing significant or unnecessary toxic effects (often expressed as maximum tolerable dose, MTD). To obtain or maintain the effective amount, the agent or composition comprising the agent may be administered as a single dose or in multiple doses. The effective amount may further vary depending on the severity of the condition that needs to be treated; this may depend on the overall health and physical condition of the mammal or patient and usually the treating doctor's or physician's assessment will be required to establish what is the effective amount. The effective amount may further be obtained by a combination of different types of contacting or administration.

The aspects and embodiments described above in general may comprise the administration of one or more therapeutic compounds to a mammal in need thereof, i.e., harboring a tumor, cancer or neoplasm in need of treatment. In general a (therapeutically) effective amount of (a) therapeutic compound(s) is administered to the mammal in need thereof in order to obtain the described clinical response(s). “Administering” means any mode of contacting that results in interaction between an agent (e.g. a therapeutic compound) or composition comprising the agent (such as a medicament or pharmaceutical composition) and an object (e.g. cell, tissue, organ, body lumen) with which said agent or composition is contacted. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent or composition comprising the agent to the object.

### Computer/Computer System

A computer or computer system as mentioned herein may utilize one or more subsystems. A computer or computer system may be a single computer apparatus comprising the one or more subsystems (e.g. internal components), or may be multiple computers or multiple computer apparatuses each being a subsystem, and optionally, each comprising one or more own subsystems. Desktops, laptops, mainframe servers, tablets, mobile phones etc. all are computers or computer systems. The subsystems are usually interconnected and include a (central) processor (single-core processor, multi-core processor on a same integrated chip, or multiple processing units on a single circuit board or networked) capable of executing instructions, an input/output (I/O) controller, and a storage device (external, internal, peripheral, cloud, any medium readable by a computer or computer system). Input devices include keyboards, scanners, a computer mouse, camera, microphone, etc. In particular, the input device is a data collection or data generating device (which by itself may comprise a computer or computer system), such as a polynucleotide sequencing device (whether automated or not). Collected or generated data are fed to a computer or computer system designed to analyze the collected or generated data; this may be an ordinary computer system on which data analyzing software is installed (on a storage device) or which is capable of accessing data analyzing software (e.g. installed in or transmitted from a network) and whereby the processor of the computer system is instructed by the data analysis software on how to process the collected or generated data fed to the computer system, and how to display these via a display adapter to an output device. Output devices are further subsystems and comprise printers, monitors, computer readable medium. Input and output devices are usually connected to a computer or computer system via input/output ports to one another or via a network.

The specific combination of hardware and software allows implementation of e.g. analysis of data generated by a polynucleotide sequencing device or expression analysis device. Different software packages (proprietary or open source) can be run on a computer or computer system to achieve the desired degree of data analysis. Output of one computerized data analysis can be the input of a subsequent computerized data analysis step, hence creating an analysis pipeline. Software components can be written in different codes (e.g. Java, C, C++, Perl, Python) as long as the computer processor is able to execute the functions of the software component.

The methods of the invention may be computer-implemented methods, or methods that are assisted or supported by a computer or by a computer system. For instance, information reflecting the analysis, determination, detection, presence or absence of DNA methylation, or of determining, detecting, assaying, assessing or analyzing biomarker expression or biomarker expression levels obtained from a sample is received by at least one first processor, and/or information reflecting the analysis, determination, detection, presence or absence of DNA methylation, or of determining, detecting, assaying, assessing or analyzing biomarker expression or biomarker expression levels obtained from a sample is provided in user readable format by at least one/another processor. The same or a further processor may be calculating a relative DNA methylation (such as relative to a control or standard), or a relative biomarker expression or biomarker expression level (such as relative to a control or standard) from the information received. The one or more processors may be coupled to random access memory operating under control of or in conjunction with a computer operating system. The processors may be included in one or more servers, clusters, or other computers or hardware resources, or may be implemented using cloud-based resources. The operating system may be, for example, a distribution of the Linux™ operating system, the Unix™ operating system, or other open-source or proprietary operating system or platform. Processors may communicate with data storage devices, such as a database stored on a hard drive or drive array, to access or store program instructions other data. Processors may further communicate via a network interface, which in turn may communicate via the one or more networks, such as the Internet or other public or private networks, such that a query or other request may be received from a client, or other device or service. Such computer-implemented methods (or such methods that are assisted or supported by a computer) may be provided as a kit or as part of a kit. The bioinformatics software required to perform (part of) the computer-implemented methods, i.e. a computer program product, may also be part of a kit, or may be provided as an individual product. A computer product may also consist of a computer readable medium which is storing any of the instructions, computer program, or bioinformatics software enabling a computer system to perform at least one of the analysis of the herein described methods and/or to perform at least one calculation (of DNA methylation or of biomarker expression or biomarker expression level) as described herein.

### Other Definitions

The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, New York (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims The content of the documents cited herein are incorporated by reference.

### EXAMPLES

### Example 1. Materials and Methods

### 1.1. Patient Population and Clinical Study

For the identification of biomarkers predicting response to anti-PD1 in early diagnosed breast (BC) cancer patients, paired pre- and on-treatment tumor biopsies from patients participating in the BioKey trial (Bassez et al. 2021, Nat Med 27:820-832) were extensively studied and characterized. The primary goal of the BioKey study was to evaluate whether one dose of pembrolizumab was able to alter intratumoral immunity and proliferation in early breast cancer. The protocol of this single center, open-label, non-randomized phase 0 study (BioKey) was approved by the local medical ethics committee of the University Hospitals Leuven (S60100). The study was conducted according to EU legislation regarding ethical regulations and was registered online (NCT03197389). All patients provided written informed consent.

Only patients with a non-metastatic operable newly-diagnosed primary invasive carcinoma of the breast that was histologically confirmed as ER⁻/PR⁻ or ER⁺ BC with a primary tumor size >1 cm, measured by any clinical examination, including mammography, ultrasound or magnetic resonance imaging, were included. The study consisted of 2 cohorts. A first cohort (the discovery cohort) involved patients scheduled for upfront surgery. Patients had either triple-negative breast cancer (TNBC), HER2⁺ (ER⁻/PR⁻), or ER⁺/PR^{+/-} (HER2^{+/-}) breast cancer (BC). A second cohort (the validation cohort) consisted of patients who received neoadjuvant chemotherapy and who had clear signs of residual tumor on imaging after 3 months of neoadjuvant chemotherapy (i.e., estimated residual tumor size of at least 10 mm). Patients either had TNBC, ER⁻/PR⁻/HER2⁺ tumors or ER⁺/PR^{+/-}/HER2^{+/-} tumors. Chemotherapy was combined with anti-HER2 therapy if the tumor was HER2*. In both cohorts, a single dose of 200 mg pembrolizumab (Keytruda® or anti-PD1) was delivered prior to surgery in a window-of-opportunity setting. Fresh tumor tissue was collected from these patients before (by needle biopsy) and 7-15 (9±2) days after (by resection) pembrolizumab.

Overall, we analyzed 29 patients form the first cohort (treatment-naive early BC) and 11 patients from the second cohort (BC treated with neoadjuvant chemotherapy) by scRNA-seq and scTCR-seq.

### 1.2. Sample Collection and Processing

Biopsies were obtained via diagnostic needle biopsy with either a 14 G or 18 G needle (pre-treatment) or surgical resection (on-treatment) and were immediately subjected to single-cell dissociation on ice. From each patient at least two cylinders were collected, of which one was fixed in formalin and embedded in paraffine for standard histopathology assessment and of which one was processed for subsequent single cell RNA (scRNA)-sequencing analysis. Briefly, the tissue samples were first mechanically dissociated using a scalpel, and subsequently enzymatically dissociated in digestion medium (2 mg/mL Collagenase P (Sigma Aldrich) and 0.2 mg/ml DNAse I (Roche) in DMEM (Thermofisher scientific)). Red blood cells were removed from the cell suspension using red blood cell lysis buffer (Roche), and cells were filtered using a 40 μm Flowmi tipstrainer (VWR). Next, the number of living cells were determined using a LUNA automated cell counter (Logos Biosystems).

### 1.3. Single-Cell RNA-Sequencing and T-Cell Repertoire (TCR) Profiling

We performed single-cell T-cell receptor sequencing (TCR-seq) and 5′ gene expression profiling on the same single-cell suspension using the Chromium™ Single Cell V(D)J Solution from 10× Genomics according to the manufacturer's instructions. Up to 5,000 cells were loaded on a 10× Genomics cartridge for each sample. Cell-barcoded 5′ gene expression libraries were sequenced on an Illumina NextSeq or/and NovaSeq6000, and mapped to the GRCh38 human reference genome using CellRanger (10× Genomics). V(D)J enriched libraries were sequenced on an Illumina HiSeq4000 and TCR alignment and annotation was achieved with CellRanger VDJ (10× Genomics). Raw gene expression matrices were generated using CellRanger (10× Genomics) and analysed using the Seurat v3 R package (Stuart et al. 2019, Cell 177:1888-1902; Butler et al. 2018, Nat Biotechnol 36:411-420). All cells expressing <200 or >6000 genes were removed, as well as cells that contained <400 unique molecular identifiers (UMIs) and >15% mitochondrial counts. Samples were merged and normalized.

### 1.4. Assessing the TCR Repertoire Using V(D)J Analysis

We only considered productive TCRs, meaning that they could be joined in the proper reading frame by V(D)J recombination without premature stop codons, enabling expression of a complete TCRα or β chain for downstream analysis. TCR clonotypes were defined as TCRs with the same complementarity-determining region 3 (CDR3) nucleotide sequences. A threshold of >2 or >5 cells with the same TCR sequence was used to define clonal cells. Clonality was defined as the complement of evenness (i.e., 1-evenness) as previously described (Riaz et al. 2017, Cell 171:934-949), where evenness represents the normalized Shannon entropy.

The evenness value lies between 0 and 1, with a high value indicating a more equal distribution of TCRs while a low value indicates TCR skewing due to clonal expansion. Clonality, which reflects the dominance of particular clones across the TCR repertoire, was calculated per sample. TCR richness (Zhu et al. 2015, Oncoimmunology 4:e1051922), defined as the number of unique TCRs divided by the total number of cells with a unique TCR, was calculated to assess clonotype diversity. The Gini coefficient or Gini index (measuring inequality among values of a frequency distribution; a Gini index of zero indicates perfect equality whereas a Gini index of one indicates maximal equality) was calculated using the ineq (v0.2-13) package in R and was assessed as an alternative measure to calculate equality of the T-cell clonotype distribution. This value ranges between 0 and 1, and the closer it is to 1 the less equal the distribution of clonotypes is (Thomas et al. 2013, Proc Natl Acad Sci USA 110: 1839-1844).

### 1.5. Clonotype Expansion and Contraction

By considering the TCR sequences of T-cell clonotypes pre- and on-treatment, we considered a clonotype being a clonotype undergoing expansion when i) there was an increase in frequency (i.e., number of cells with same TCR) or proportion (i.e., frequency normalized for number of cells in a sample with a TCR detected) on-treatment versus pre-treatment and ii) a frequency on-treatment of >2. The number of expanded clonotypes upon treatment defined by these two criteria was calculated per patient and had to be >30 to define clonotype expansion. An additional more stringent criterion was applied requiring a clonotype to i) increase in frequency on-treatment versus pre-treatment and ii) a frequency on-treatment of >5. A threshold of 10 expanded clonotypes defined by this criterion identified patients with clonotype expansion. Clonotypes not detected pre-treatment, but clonal on-treatment (frequency of >2 or of >5) were also considered as expanded clonotypes. Eight out of 29 patients in the discovery cohort showed clear clonotype expansion based on these 3 definitions, while this was the case for three out of 11 patients in the validation cohort. These patients were designated as patients with clonotype expansion or Es (also referred to as “expanders”), while the others were designated as patient with no or limited clonotype expansion or NEs (also referred to as “non-expanders”).

Because anti-PD1 was delivered in a window-of-opportunity setting, we could not explore whether T cell expansion translated into clinical benefit. However, in melanoma, peripheral T cell expansion occurring within three weeks after start of treatment correlates with improved clinical response to ICB six months later (Fairfax et al. 2020, Nat Med 26:193-199; Valpione et al. 2020, Nat Cancer 1:210-221). Because the number of cancer cells decreased on-treatment in Es, this suggests that BC T cell expansion might also be associated with clinical benefit in BC. We additionally observed that the majority of expanding T cell clonotypes were already detected pre-treatment.

1.6. scRNA-Seq Clustering Leading to Cell Types

Default parameters of Seurat were used, unless mentioned otherwise. Briefly, for the clustering of all cell types, 2000 variable genes were identified and principal component analysis (PCA) was applied to the dataset to reduce dimensionality after regressing for number of UMIs (counts; UMI=unique molecular identified), percentage mitochondrial genes and cell cycle (S and G2M scores calculated by the Cel/CycleScoring function in Seurat). The 20 most informative principal components (PCs) were used for clustering and Uniform Manifold Approximation and Projection for Dimension Reduction (UMAP; McInnes et al. 2018, https://arxiv.org/pdf/1802.03426.pdf; Becht et al. 2019, Nat Biotechnol 37:38-44) reduction. Clusters in the resulting two-dimensional UMAP representation consisted of distinct cell types, which were identified based on the expression of marker genes. Differentially-expressed genes (DEGs) that functionally characterized the clusters were defined by the Model-based Analysis of Single-cell Transcriptomics (MAST) test implemented in the FindAllMarkers function from Seurat.

1.7. scRNA-Seq Clustering Leading to Cell Subtypes

To subcluster T-cells from pre-treatment and on-treatment samples we used the integration pipeline of Seurat. We regressed for the following confounding factors: number of UMIs (counts), percentage of mitochondrial genes, individual patient, cell cycle and interferon response score (calculated by the AddModuleScore function in Seurat using the gene set BROWNE_INTERFERON_RESPONSIVE_GENES from the Molecular Signatures Database or MSigDB v6.2). For myeloid cells, a specific clustering approach was used: first, myeloid cell subclustering identified cDC subclusters based on marker gene expression (cCDs: CLEC9A, XCR1, CD1C, CCR7, CCL17, CCL19; Langerhans-like: CD1A, CD207), as previously described (Qian et al. 2020, Cell Res 30:745-762). These DC clusters were excluded from subsequent subclustering efforts, but instead were merged with pDCs for subclustering of dendritic cells, using a similar approach as for T-cells with an exception that regression for interferon response was not needed. All phenotypes were shared between patients, pre-treatment versus on-treatment and between breast cancer (BC) subtypes.

In the second cohort of BC patients receiving neoadjuvant chemotherapy followed by one dose of pembrolizumab, the same clustering approach was used. However, to subcluster T-cells, we used the label transfer pipeline from Seurat. Briefly, the annotation of T-cells performed in the first cohort served as a reference dataset to assign T-cells from the queried data to a given T-cell phenotype.

### 1.8. Class Prediction Using Prediction Analysis of Microarrays (PAM)

To identify an optimal set of biomarkers predictive for response to anti-PD1 in early breast cancer, prediction analyses of microarrays (PAM) analysis (Tibshirani et al. 2002, Proc Natl Acad Sci USA 99:6567-72), based on nearest shrunken centroid methodology, was performed. The R package pamr (version 1.56.1) was used. PAM using 8-fold cross-validation was performed on the pre-treatment samples. Two different PAM analyses were performed, and each analysis was repeated 10 times to identify robust markers. One analysis included all TCR measures (i.e., clonality, richness and Gini index) and relative abundance of cell (sub)types per patient as input, while the other analysis included the average expression of all genes in all pre-treatment cells per patient as input. Since the same number of genes is required as input in PAM to predict the accuracy of the identified gene-signature from the discovery cohort in the validation cohort (with pamr.predict function), only genes overlapping in both cohorts were used as input.

### 1.9. Unsupervised Hierarchical Clustering

Unsupervised complete-linkage hierarchical clustering, based on the complete-linkage method using Euclidian distance metric, was used to visualize the feature/sample relationship and to validate the signatures identified by PAM. The results were visualized as a 2-dimensional heatmap with 1 dendrogram classifying patients into different groups. Expansion status (expanded, E; or non-expanded, NE) and breast cancer type (ER⁺, HER2⁺ or TNBC) were indicated in different annotation colors above the heatmap. The R package ComplexHeatmap version 2.2.0 was used.

### 1.10. Generalized Logistic Regression Analysis

To assess the predictive value of the signatures obtained with PAM analyses in an independent analysis, individual signature values were combined into a single predictor and corresponding ROC (receiver operating characteristics) curve by fitting a logistic regression model with ranks of the values. Optimism of the AUC (area under the ROC curve) value of the combined predictor was estimated using 10 (validation cohort) and 50 (discovery cohort and for both cohorts combined) non-parametric bootstrap iterations and subtracted to obtain an unbiased estimate of performance. This method was also used to assess whether smaller combinations within the minimal signatures obtained with PAM analyses could be predictive. ROC curves were created with R package pROC version 1.16.2.

### 1.11. Bayesian Generalized Linear Regression Analysis

To assess the predictive value of the signatures obtained with PAM analyses in an independent analysis, individual signature values were combined into a single predictor or combined signature score and corresponding ROC (receiver operating characteristics) curve by fitting a Bayesian generalized linear regression model with the values. Optimism of the AUC (area under the ROC curve) value of the combined signature scores was estimated using 10 (validation cohort) and 50 (discovery cohort and for both cohorts combined) non-parametric bootstrap iterations and subtracted to obtain an unbiased estimate of performance (i.e., corrected AUC values). The models fitted on the discovery cohort data, were used to predict combined signature scores for the validation cohort and validation cohort combined with discovery cohort. This method was also used to assess whether smaller combinations within the minimal signatures obtained with PAM analyses could be predictive. ROC curves were created with R package pROC version 1.16.2.

### Example 2. Variables and Gene Expression Markers Predicting T-Cell Expansion in Breast Cancer when Treated with an Immunotherapeutic Agent

To identify an optimal set of biomarkers predictive for response to anti-PD1 in early breast cancer, PAM using 8-fold cross validation was performed on the pre-treatment samples from the discovery cohort (n=28; 20 non-expanders (NEs) and 8 expanders (Es)). Two different PAM analyses were performed: A) the first analysis included all TCR measures (i.e., clonality, richness and Gini index) and relative abundance of cell (sub)types at baseline per patient as input, and B) the second analysis included the average expression of all genes in all pre-treatment cells per patient as input. The gene signatures were validated in the validation cohort.

### 2.1. Variables Predicting T-Cell Expansion in Breast Cancer

For the PAM analysis using all features including TCR measures and relative frequencies of cell (sub)types as input, a minimal set of 6 features was identified that classified Es from NEs before treatment with an accuracy of 100% (Table 1). To confirm the robustness of this signature, this analysis was repeated 10 times, and all analyses gave comparable results with the 6 features as top ranked predictive markers for T-cell expansion. In 9 out of 10 cross-validations the same 6 features were identified as most predictive markers and addition of more features didn't change the sensitivity, specificity or overall accuracy. In one cross-validation, 5 features (5 out of the above 6 features) were identified as minimal set, but gave a worse prediction (sensitivity of 87.5% (7/8)).

TABLE 1
Summary of results from PAM analysis.

| Discovery cohort | | | Overall |
|---|---|---|---|
| (E = 8, NE = 20) | Sensitivity | Specificity | accuracy |
| 6 features | 100% (8/8) | 100% (20/20) | 100% (28/28) |

E, patients with clonotype expansion;
NE; patients with no or limited clonotype expansion

The 6 features predictive of T-cell expansion are:

Gini index of T-cell clonotype distribution: A measure to calculate equality of the T-cell clonotype distribution. This value ranges between 0 and 1, and the closer it is to 1 the less equal the distribution of clonotypes is (skewing of clonal TCRs; similar to clonality).

TCR Richness: TCR richness, defined as the number of unique T-cell receptors (TCRs) divided by the total number of cells with a unique TCR, was calculated to assess clonotype diversity.

T_{EX}, CD8 T_{EX}, CD4 T_{EX}: Exhausted or Experienced T-cells/Exhausted activated or Experienced activated T-cells. One CD8+ cell cluster and CD4+ cell cluster expressing PD1 were identified. These cells represent activated T cells with exhaustion-like characteristics based on expression of immune-checkpoint (LAG3, HAVCR2, PDCD1), effector (IFNG, NKG7) and cytotoxic (GZMB, PRF1) markers. Collectively, these cells are referred to as experienced T cells (T_{EX} cells). Separately, these cells are referred to as CD8+ experienced T cells (CD8 T_{EX}) and CD4+ experienced T cells (CD4 T_{EX}), respectively.

C7_CX3CR1: T cell inhibitory CX3CR1+ macrophages (Hart et al. 2009, Neoplasia 11:564-573; Zheng et al. 2013, Mol Cancer 12:141). Interestingly, CX3CR1+ macrophages are ablated during ICB in a sarcoma tumor model (Gubin et al. 2018, Cell 175:1014-1030), while genetic ablation of CX3CL1, the ligand of CX3CR1, also inhibited tumor growth and shifted tumor-associated macrophages towards an anti-tumor M1 phenotype (Korbecki et al. 2020, Int J Mol Sci 21:3723). C7_CX3CR1 macrophages also expressed complement C3, which suppresses the infiltration and function of CD8+ and CD4+ T cells 51,52. Overall, this suggests that inhibition of both CX3CR1 or C3 might be therapeutically beneficial.

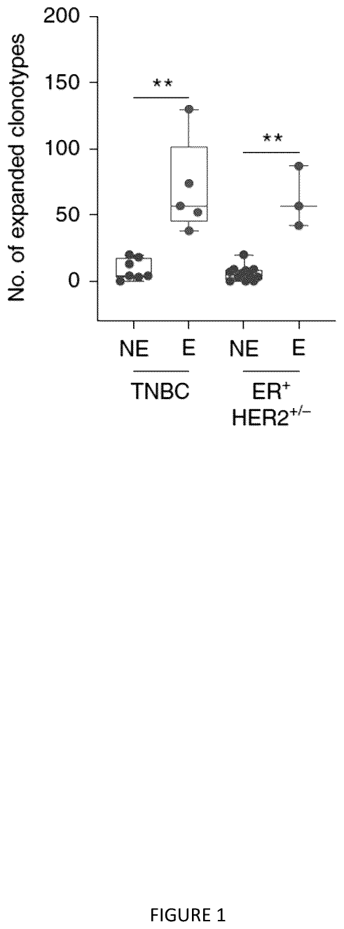

T cell expansion was observed independent of the breast cancer subtype as is illustrated in FIGS. 1A and 1B.

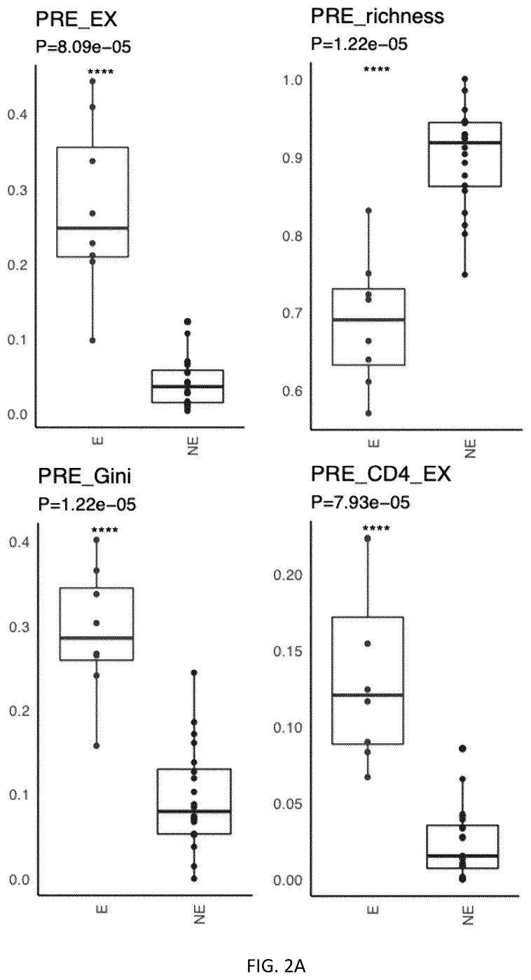

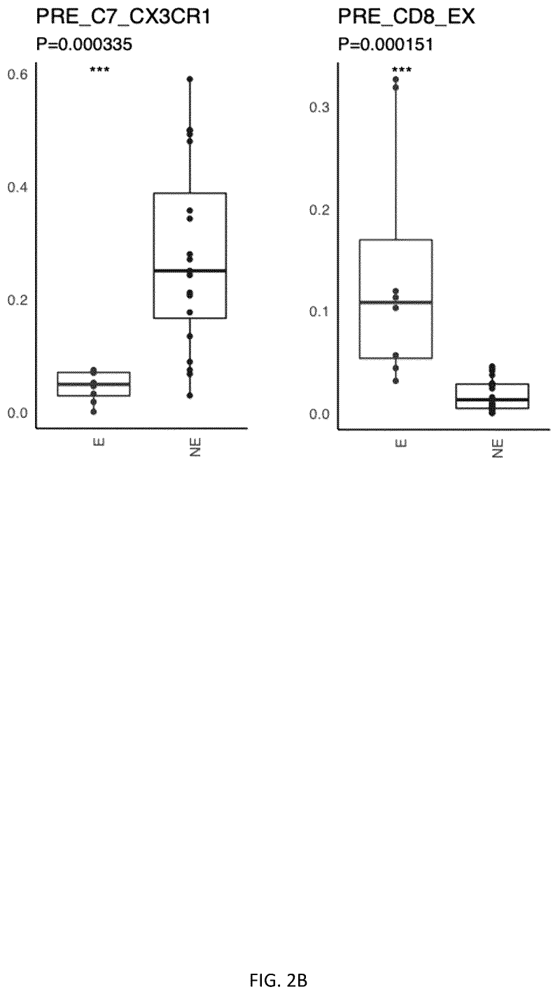

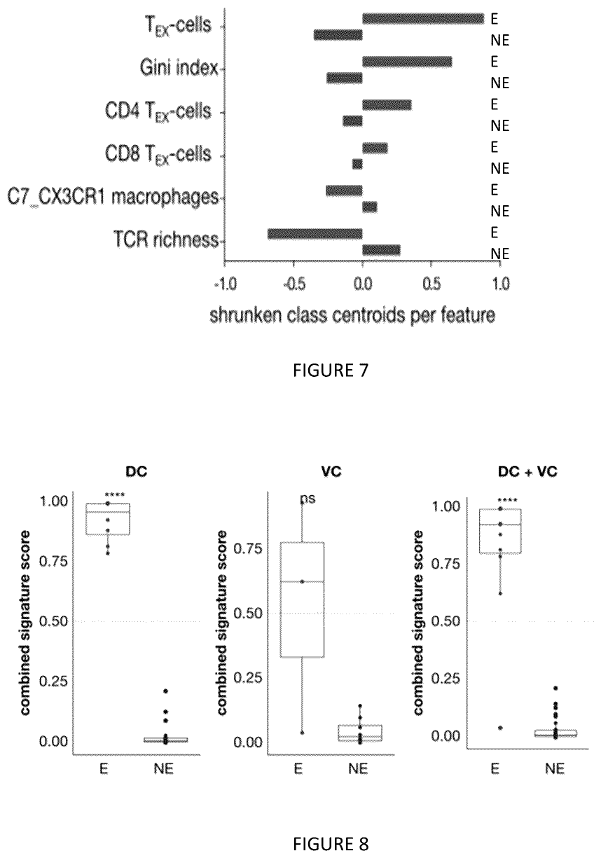

Four of the features predictive of T-cell expansion have a value higher in Expanders compared to Non-Expanders: T_{EX}, CD8 T_{EX}, CD4 T_{EX} and the Gini-index of T-cell clonotype distribution. On the contrary, two of the features predictive of T-cell expansion have a value higher in Non-Expanders compared to Expanders: TCR richness, and C7_CX3CR1 macrophages. This is depicted in FIGS. 2A, 2B, and 7.

To validate the 6-feature signature, unsupervised hierarchical clustering of the pre-treatment samples from the discovery cohort was applied based on the individual feature values. The clustering analysis showed two major clusters: all Es clustered together in one group and all NEs clustered in the other group (not shown).

We also performed an independent analysis in which we tested the predictive potential of the individual features. The resulting AUC values are given in Table 2.

TABLE 2

| feature | AUC |
|---|---|
| TCR richness | 0.97 |
| C7_CX3CR1 macrophages | 0.94 |
| CD8 T_{EX} | 0.97 |
| CD4 T_{EX} | 0.99 |
| Gini index of T-cell clonotype distribution | 0.97 |
| T_{EX} | 0.99 |

Only 5 out of 6 features were calculated in the validation cohort, since macrophage subclusters (C7_CX3CR1) were not annotated for the latter, making it impossible to validate the signature in the validation cohort via PAM analysis since for the latter the same input values are required. We therefore performed an independent analysis to test the predictive potential of our 6-feature signature. Bayesian generalized linear regression models were fitted to combine the individual feature values into a single predictor value/combined signature score. By replacing the missing values for the macrophage feature by zero, we were capable of predicting a combined signature score (i.e., single predictor value representing all the features) per patient in the validation cohort with this model. Fitting of the Bayesian model on the discovery cohort led to combined signature scores that could clearly separate Es from NE (FIG. 8) and to AUC values, both corrected (bootstrap) and uncorrected of 1. Next, the Bayesian model that was built on the discovery cohort was used to derive combined signature scores per patient for the validation cohort and the combination of the discovery and validation cohort. All, but one patient could be correctly classified based on the single predictor values of the feature signature. The combined signature scores led to corrected AUC values of 0.81 and 0.97, respectively for the validation cohort and the combination of both cohorts.

Combinations of the 6 features or less were likewise analyzed to test their predictive potential: generalized logistic regression models and Bayesian generalized linear regression models were fitted to combine individual feature values into a single predictor (see Methods). Combining the 6 features gave an AUC value of 1 for the discovery cohort, both corrected and uncorrected. Interestingly, corrected and uncorrected AUC values of 1 were also obtained when combining 5, 4, 3 or 2 features. Combinations that were tested included i) TCR richness, C7_CX3CR1 macrophages, CD8 T_{EX}, CD4 T_{EX}, Gini index of T-cell clonotype distribution, and T_{EX}; ii) C7_CX3CR1 macrophages, CD8 T_{EX}, CD4 T_{EX}, Gini index of T-cell clonotype distribution, and T_{EX}; iii) CD8 T_{EX}, CD4 T_{EX}, Gini index of T-cell clonotype distribution, and T_{EX}; iv) CD4 T_{EX}, Gini index of T-cell clonotype distribution, and T_{EX}; and v) Gini index of T-cell clonotype distribution, and T_{EX}.

### 2.2. Genes Predicting T-Cell Expansion in Breast Cancer

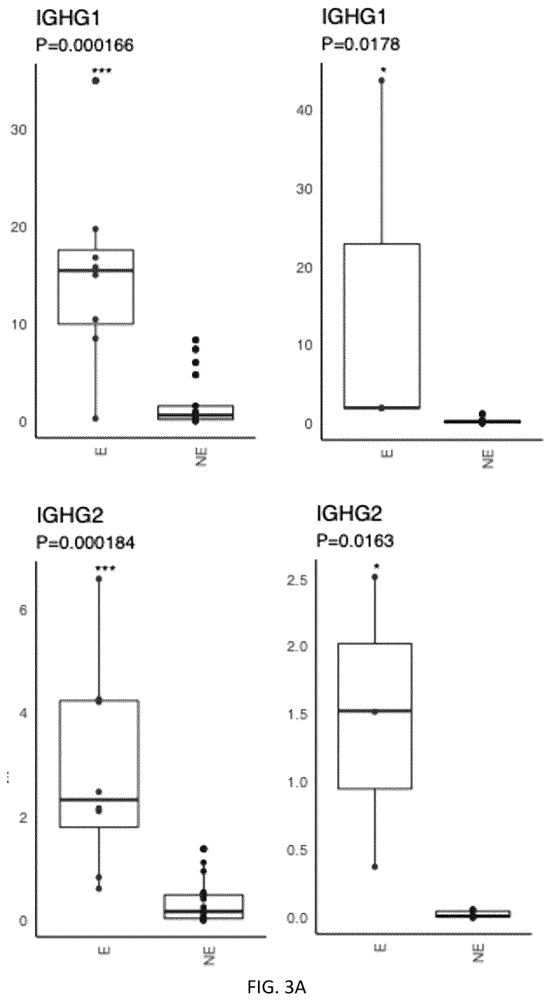

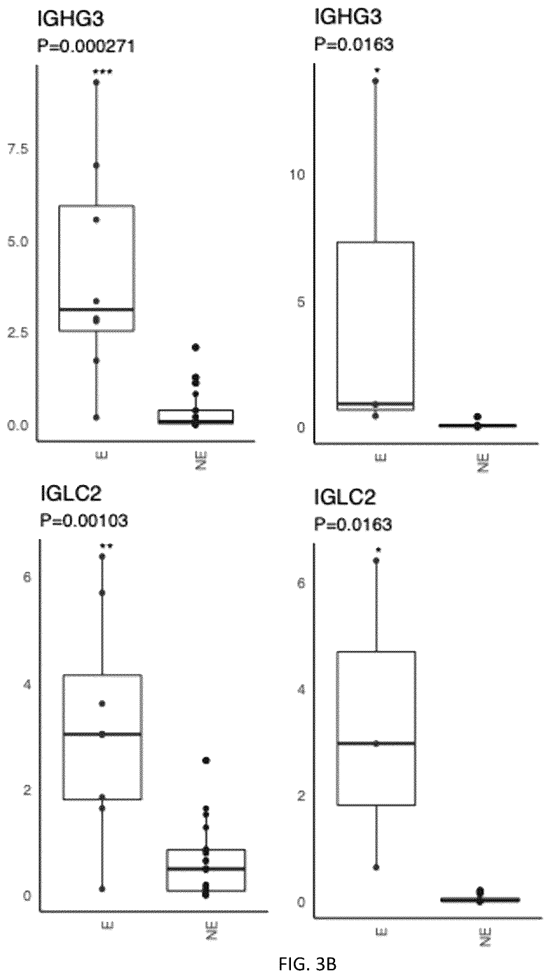

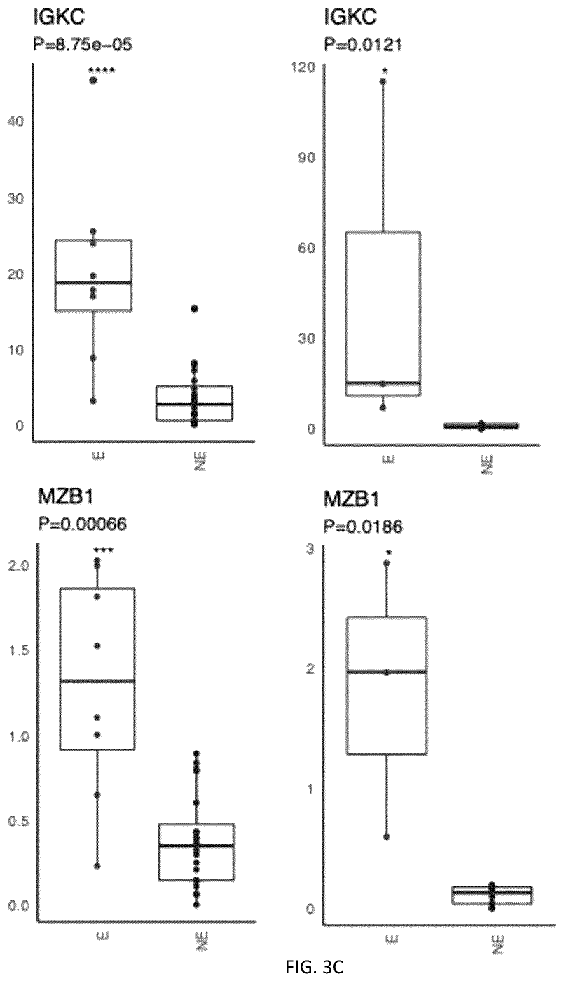

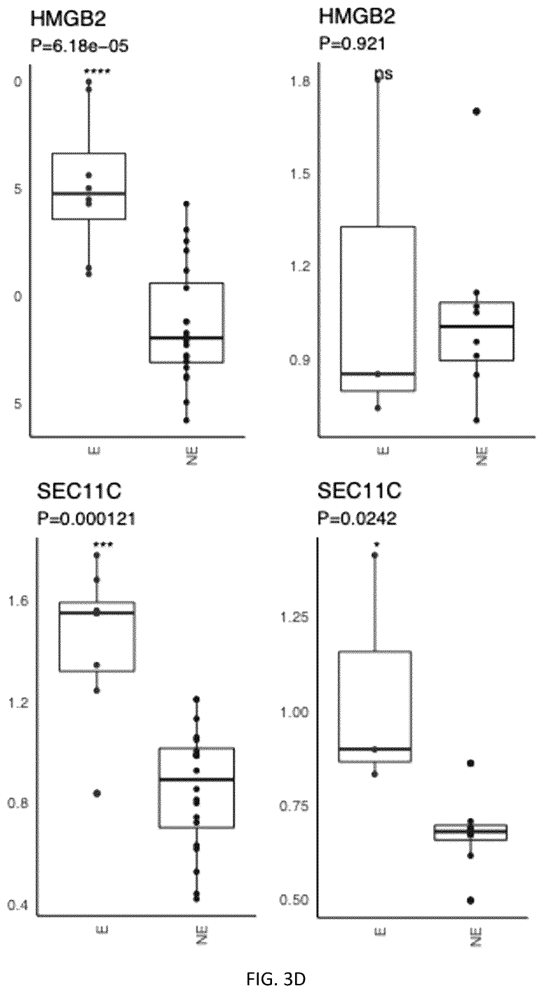

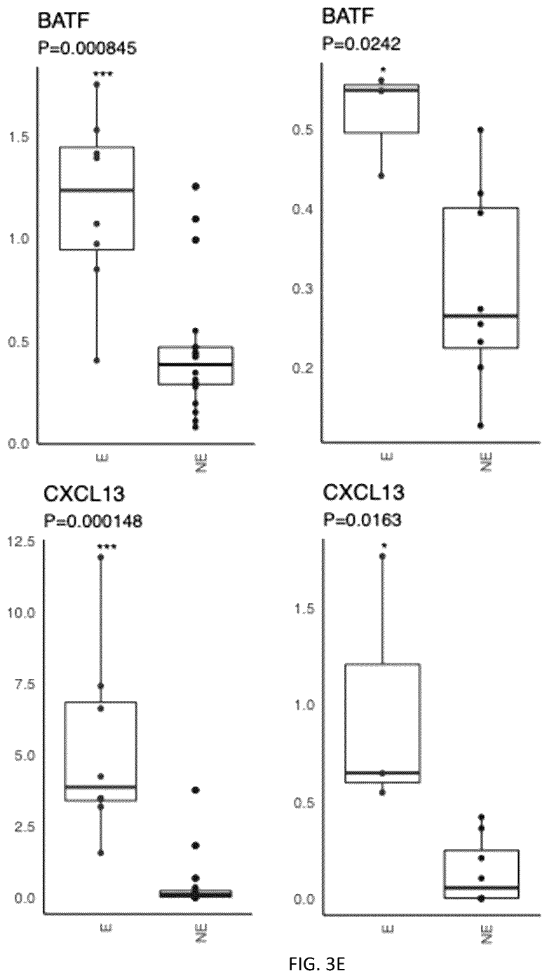

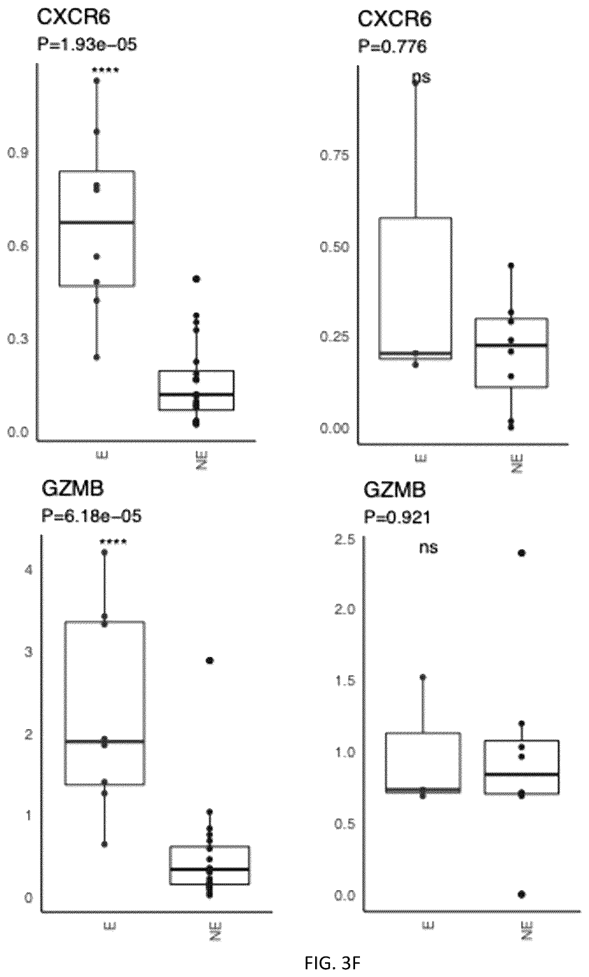

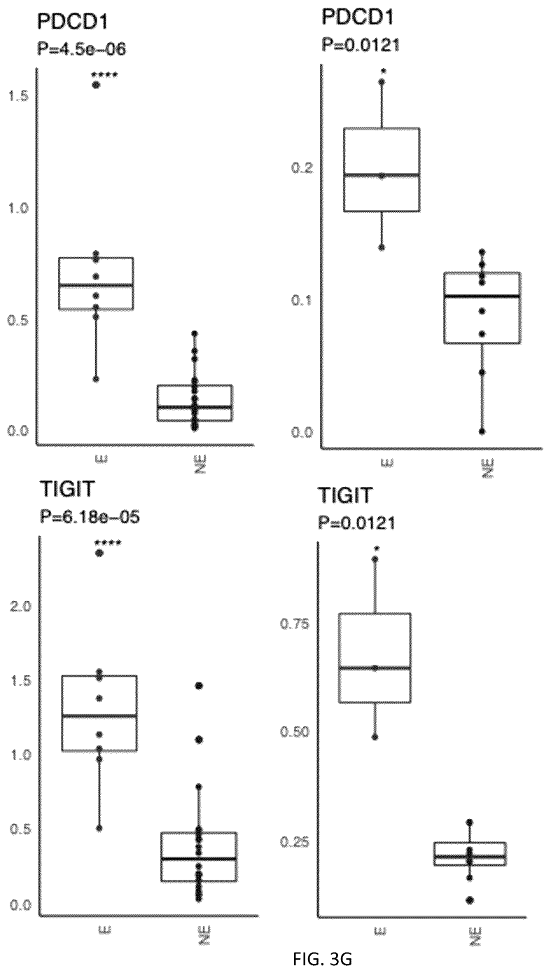

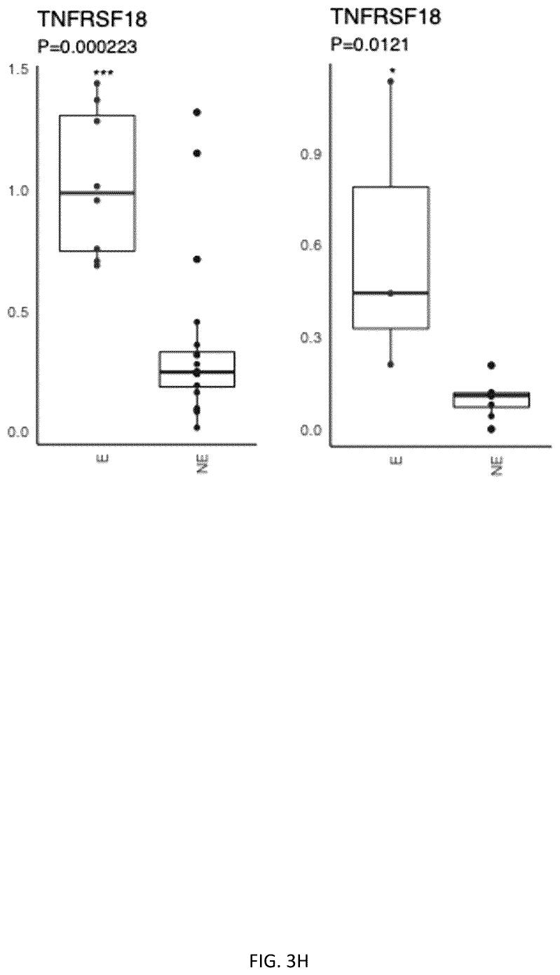

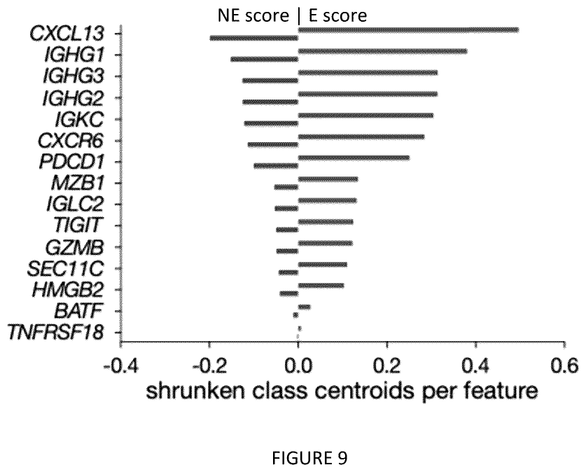

The current breast cancer scRNA-seq data were independently analyzed in order to find genes the expression of which is predictive of T cell expansion (and thus of a positive response of a breast cancer patient to the immunotherapy). As a result of the PAM analysis carried out on the average expression of all genes in all pre-treatment cells per patient in the discovery cohort, a set of 15 genes predictive of T cell expansion (and thus of a positive response of a breast cancer patient to the immunotherapy) were identified. This PAM analysis using 8-fold cross-validation was repeated 10 times, and in all analyses the same 15 genes were observed as most predictive markers and all genes were significantly upregulated in Expanders versus Non-Expanders, both in the discovery and in the validation cohorts (FIGS. 3A-3H and 9).

To validate the identified gene signatures, unsupervised hierarchical clustering was applied on the pre-treatment samples in the discovery and validation cohort. Based on the expression profile of the genes, a clear distinction between Es and NEs in each cohort was seen that was similar to the one obtained with PAM.

We also performed an independent analysis where we tested the predictive potential of the individual features. The resulting AUC values are given in Table 3 and confirm the predictive potential of the individual genes in the signature.

TABLE 3
The AUC values of the individual genes identified as predictor
of T cell expansion/positive response to immunotherapy
in breast cancer patients treated with ICB as determined
for the combined discovery and validation cohorts.

| Gene | AUC discovery + validation cohort |
|---|---|
| TIGIT | 0.94 |
| SEC11C | 0.89 |
| PDCD1 | 0.93 |
| CXCR6 | 0.90 |
| GZMB | 0.87 |
| IGKC | 0.95 |
| IGHG2 | 0.95 |
| BATF | 0.89 |
| IGHG3 | 0.95 |
| IGLC2 | 0.92 |
| MZB1 | 0.93 |
| HMGB2 | 0.84 |
| TNFRSF18 | 0.89 |
| IGHG1 | 0.93 |
| CXCL13 | 0.96 |

A number of random combinations of genes were likewise analyzed: generalized logistic regression models were fitted to combine individual feature values into a single predictor (see Methods). For such combinations, the AUC value (uncorrected and corrected) of was determined for the discovery cohort, the validation cohort, and both cohorts combined. Combinations that were tested included:

### 1) 10-Gene-Panels

TIGIT, SEC11C, PDCD1, CXCR6, GZMB, IGKC, IGHG2, BATF, IGHG3, IGLC2 (AUC combined: 1.00; AUC corrected DC/VC/DC+VC:0.94/0.71/0.90, respectively)
SEC11C, PDCD1, CXCR6, GZMB, IGKC, IGHG2, IGHG3, HMGB2, TNFRSF18, IGHG1 (AUC combined: 1.00; AUC corrected DC/VC/DC+VC:0.91/0.75/0.92, respectively)
TIGIT, PDCD1, CXCR6, GZMB, IGKC, IGHG2, IGHG3, IGLC2, MZB1, HMGB2 (AUC combined: 1.00; AUC corrected DC/VC/DC+VC:0.94/0.79/0.90, respectively)

### 2) 5-Gene Panels

IGKC, IGLC2, CXCR6, SEC11C, HMGB2 (AUC combined: 1.00; AUC corrected DC/VC/DC+VC:0.96/0.94/0.92, respectively)
CXCR6, TNFRSF18, SEC11C, IGKC, IGHG2 (AUC combined: 1.00; AUC corrected DC/VC/DC+VC:0.95/0.94/0.93, respectively)
SEC11C, IGKC, TIGIT, IGHG2, MZB1 (AUC combined: 1.00; AUC corrected DC/VC/DC+VC:0.98/1.00/0.93, respectively)
IGKC, IGHG1, IGHG2, IGHG3, CXCL13 (AUC combined DC/VC/DC+VC: 1.00/1.00/0.99, respectively; AUC corrected DC/VC/DC+VC:0.94/1.00/0.96, respectively)

### 3) 4-Gene Panels

IGHG1, IGHG2, IGHG3, CXCL13 (AUC combined DC/VC/DC+VC: 0.98/1.00/0.97, respectively; AUC corrected DC/VC/DC+VC:0.94/1.00/0.96, respectively)

### 4) 3-Gene Panels

IGLC2, GZMB, MZB1 (AUC combined DC/VC/DC+VC: 0.95/1.00/0.96, respectively; AUC corrected DC/VC/DC+VC: 0.90/0.94/0.94, respectively)
SEC11C, HMGB2, CXCL13 (AUC combined DC/VC/DC+VC: 0.99/1.00/0.96, respectively; AUC corrected DC/VC/DC+VC:0.96/0.98/0.94, respectively)
IGHG1, SEC11C, TIGIT (AUC combined DC/VC/DC+VC: 0.96/1.00/0.96, respectively; AUC corrected DC/VC/DC+VC:0.92/1.00/0.93, respectively)
IGHG1, IGHG3, CXCL13 (AUC combined DC/VC/DC+VC: 0.98/1.00/0.96, respectively; AUC corrected DC/VC/DC+VC:0.94/1.00/0.94, respectively)

### 5) 2-Gene Panels

IGHG1, CXCL13 (AUC combined DC/VC/DC+VC: 0.97/1.00/0.97, respectively; AUC corrected DC/VC/DC+VC: 0.93/1.00/0.96, respectively)

In 1) to 5), “DC” refers to discovery cohort and “VC” refers to validation cohort.

Similar as for the 6-feature signature, a Bayesian generalized regression model was fitted to combine individual gene values into single predictor values. Fitting of the Bayesian model on the discovery cohort led to combined signature scores that could clearly separate Es from NE, leading to AUC value of 1 and a bootstrap corrected AUC of 0.99. This model built on the discovery cohort was used to derive combined signature scores per patient for the validation cohort and both cohorts combined. Again, all, but one patient could be correctly classified based on the combined signature scores of the 15-gene signature. The combined signature scores led to corrected AUC values of 0.96 and 0.94, respectively for the validation cohort and the combination of both cohorts.

A number of random combinations of genes were likewise analyzed: Bayesian generalized linear regression models were fitted to combine individual feature values into a single predictor (see Methods).

For such combinations, the AUC value (uncorrected and corrected) of was determined for the discovery cohort, the validation cohort, and both cohorts combined. Combinations that were tested included:

### 6) 10-Gene-Panels

BATF, TNFRSF18, GZMB, HMGB2, IGHG2, IGKC, IGLC2, MZB1, SEC11C, TIGIT (AUC corrected DC/VC/DC+VC: 0.98/0.96/0.95, respectively)
BATF, TNFRSF18, GZMB, HMGB2, IGHG1, IGHG2, IGHG3, MZB1, SEC11C, TIGIT (AUC corrected DC/VC/DC+VC: 0.98/0.96/0.94, respectively)
BATF, TNFRSF18, CXCL13, GZMB, IGHG1, IGHG2, IGKC, PDCD1, SEC11C, TIGIT (AUC corrected DC/VC/DC+VC: 0.98/1/0.96, respectively)

### 7) 5-Gene Panels

BATF, CXCL13, IGKC, IGLC2, TIGIT (AUC corrected DC/VC/DC+VC: 0.96/1/0.96, respectively)
TNFRSF18, CXCR6, IGHG3, IGLC2, TIGIT (AUC corrected DC/VC/DC+VC: 0.95/0.96/0.92, respectively)
BATF, TNFRSF18, HMGB2, MZB1, TIGIT (AUC corrected DC/VC/DC+VC: 0.94/0.88/0.92, respectively)
CXCL13, IGHG1, IGHG2, IGHG3, IGKC (AUC corrected DC/VC/DC+VC: 0.96/1/0.96, respectively)

### 8) 4-Gene Panels

CXCL13, IGHG1, IGHG2, IGHG3 (AUC corrected DC/VC/DC+VC: 0.96/1/0.96, respectively)

### 9) 3-Gene Panels

IGLC2, PDCD1, TIGIT (AUC corrected DC/VC/DC+VC: 0.97/1/0.92, respectively)
IGHG2, IGHG3, SEC11C (AUC corrected DC/VC/DC+VC: 0.98/1/0.96, respectively)
BATF, GZMB, MZB1 (AUC corrected DC/VC/DC+VC: 0.91/1/0.93, respectively)
CXCL13, IGHG1, IGHG3 (AUC corrected DC/VC/DC+VC: 0.96/1/0.96, respectively)

### 10) 2-Gene Panels

CXCL13, IGHG1 (AUC corrected DC/VC/DC+VC: 0.95/1/0.93, respectively)

In 6) to 10), “DC” refers to discovery cohort and “VC” refers to validation cohort.

In as far as the inventors are aware, scRNA sequencing datasets other than the dataset currently relied on are not publicly available for breast cancer treated with an immunotherapeutic agent. Such datasets are available for melanoma, more particularly as published by Hugo et al. 2016 (Cell 165:35-44) and Liu et al. 2019 (Nat Med 25:1916-1927). The markers identified in WO2018/209324 and WO2020/205644 likewise appear to be based on a dataset obtained from melanoma patients treated with an immunotherapeutic agent. The inventors hence next assessed whether the 15 genes identified to predict T-cell expansion in breast cancer patients treated with an immunotherapeutic agent (and thus of a positive response of a breast cancer patient to the immunotherapy) were likewise predictive of positive response of a melanoma cancer patient to the immunotherapy. Hence, the data as published by Hugo et al. 2016 (Cell 165:35-44) and Liu et al. 2019 (Nat Med 25:1916-1927) were analyzed similarly as the current breast cancer data and predictive power was expressed as AUC values. The “Liu” dataset (Liu et al. 2019, Nat Med 25:1916-1927) was analyzed integrally (whole data set) and partially (only data relating to CTLA4-naïve patients). The “Hugo” dataset (Hugo et al. 2016, Cell 165:35-44) was analyzed integrally.

The results are summarized in Table 4.

Unexpectedly and surprisingly, none of the 15 genes identified herein as predictive of T-cell expansion in breast cancer patients treated with an immunotherapeutic agent (and thus of a positive response of a breast cancer patient to the immunotherapy) is performing well in predicting a positive response of a melanoma cancer patient to the immunotherapy. The latter is reflected in low AUC values (highest AUC values: 0.64 for HMGB2 in the Hugo melanoma dataset; 0.63 and 0.58 for BATF in both Liu melanoma datasets), compared to the high AUC values in the breast cancer setting (lowest AUC value 0.84 for HMGB2).

TABLE 4
AUC values of the individual genes identified as predictor of T
cell expansion/positive response to immunotherapy in breast cancer
patients treated with ICB as determined for the combined discovery
and validation cohort (as in Table 4); and AUC values of the same
genes in relation to response to ICB of melanoma patients.

| | AUC | | melanoma | |
|---|---|---|---|---|
| | discovery + | | Liu - | |
| | validation | melanoma | CTLA4 | melanoma |
| Gene | cohort | Liu | naive | Hugo |
| TIGIT | 0.94 | 0.60 | 0.55 | 0.51 |
| SEC11C | 0.89 | 0.52 | 0.54 | 0.59 |
| PDCD1 | 0.93 | 0.53 | 0.50 | 0.55 |
| CXCR6 | 0.90 | 0.57 | 0.49 | 0.57 |
| GZMB | 0.87 | 0.56 | 0.54 | 0.59 |
| IGKC | 0.95 | 0.52 | 0.55 | 0.53 |
| IGHG2 | 0.95 | 0.57 | 0.49 | 0.53 |
| BATF | 0.89 | 0.63 | 0.58 | 0.54 |
| IGHG3 | 0.95 | 0.59 | 0.53 | 0.41 |
| IGLC2 | 0.92 | 0.46 | 0.55 | 0.47 |
| MZB1 | 0.93 | 0.58 | 0.52 | 0.47 |
| HMGB2 | 0.84 | 0.56 | 0.46 | 0.64 |
| TNFRSF18 | 0.89 | 0.58 | 0.51 | 0.58 |
| IGHG1 | 0.93 | 0.54 | 0.55 | 0.49 |
| CXCL13 | 0.96 | 0.55 | 0.52 | 0.49 |

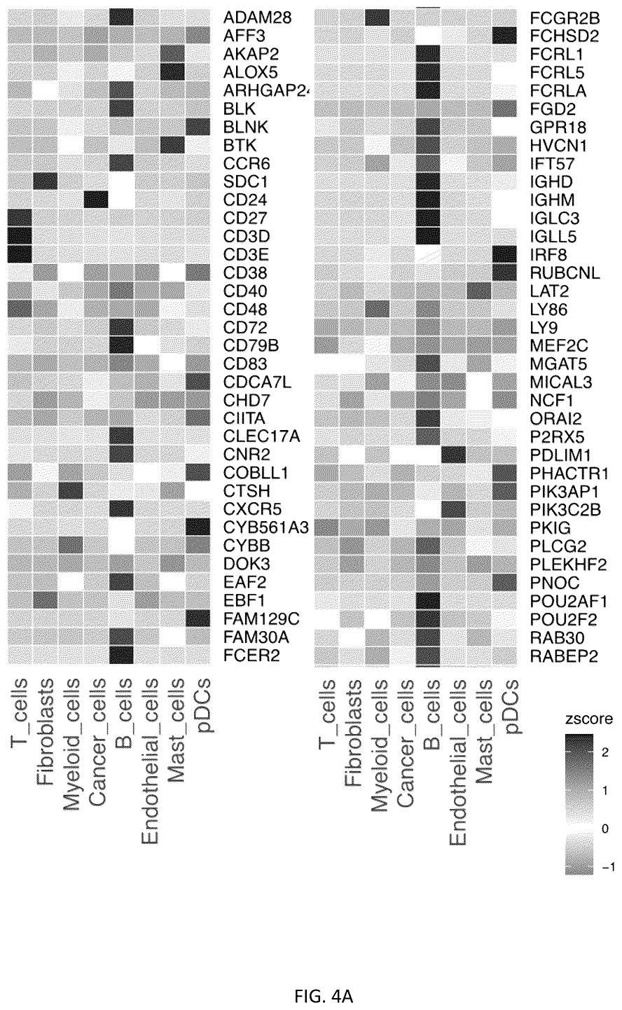

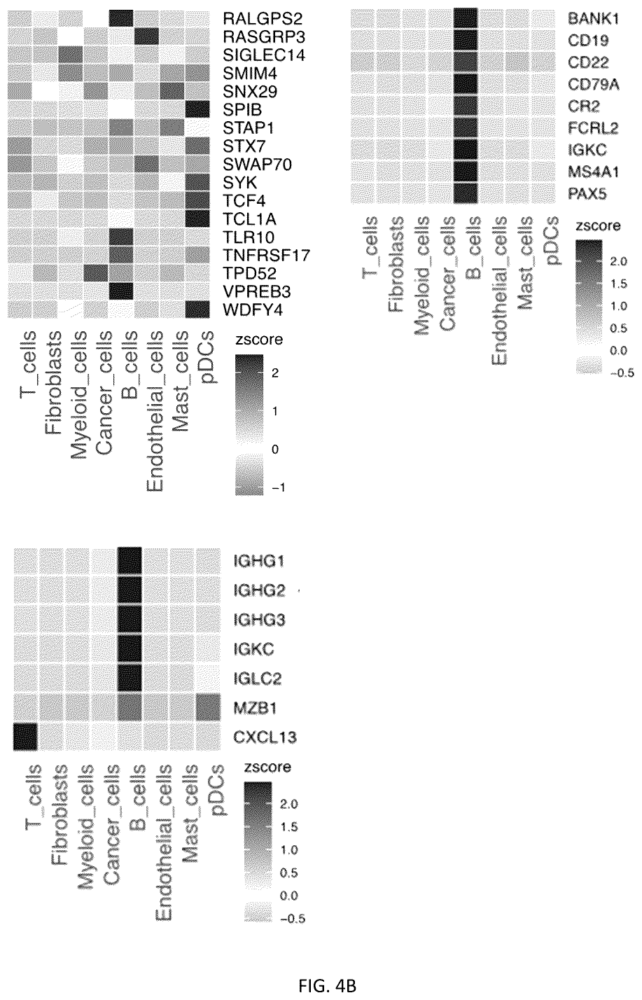

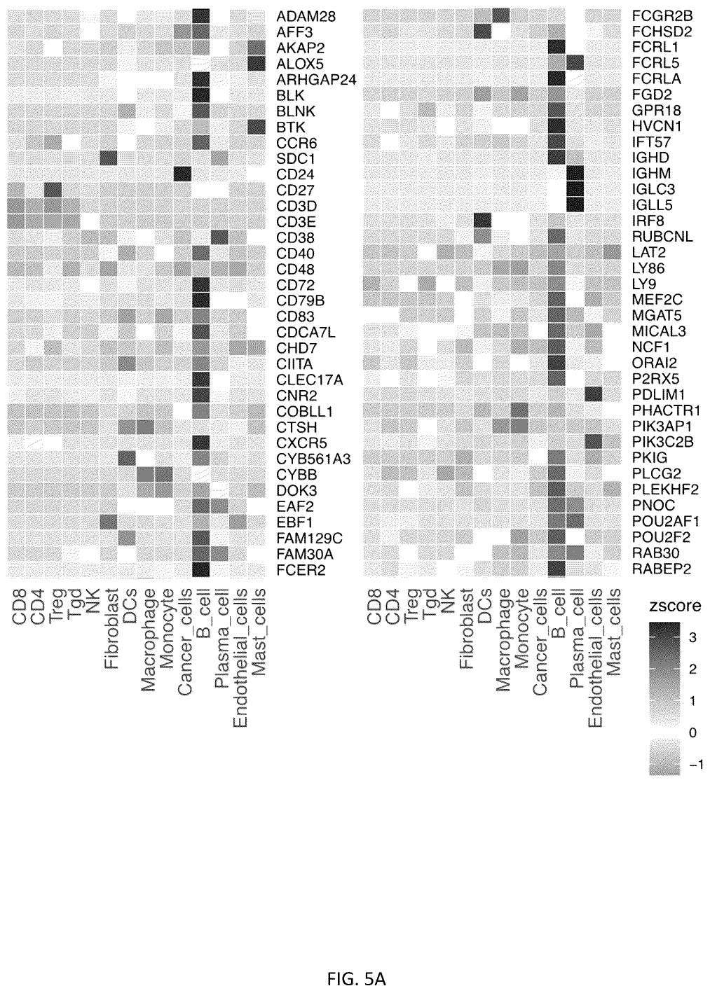

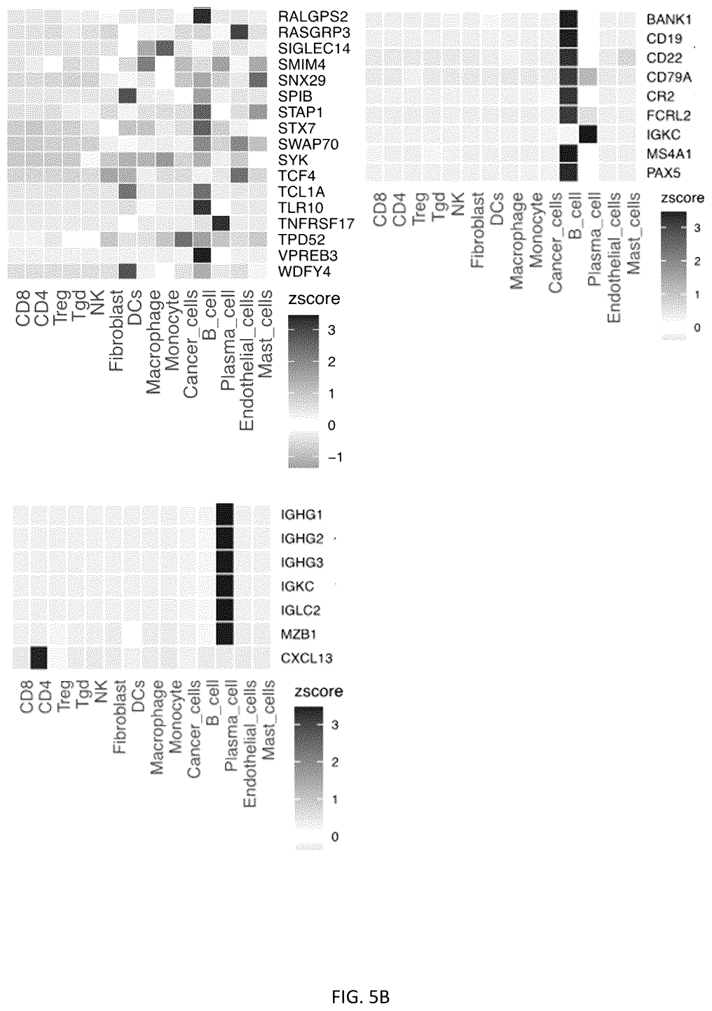

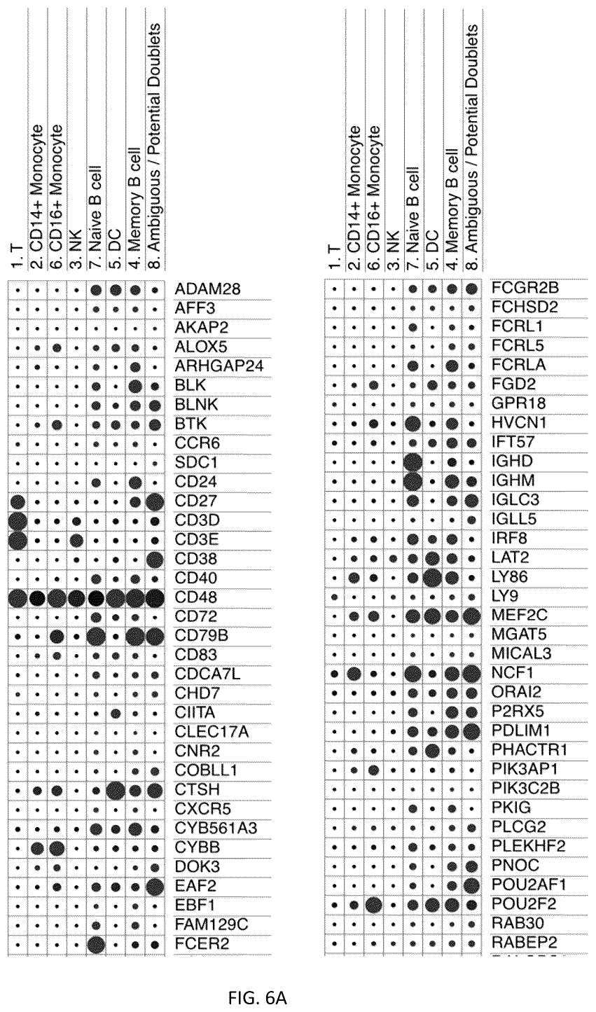

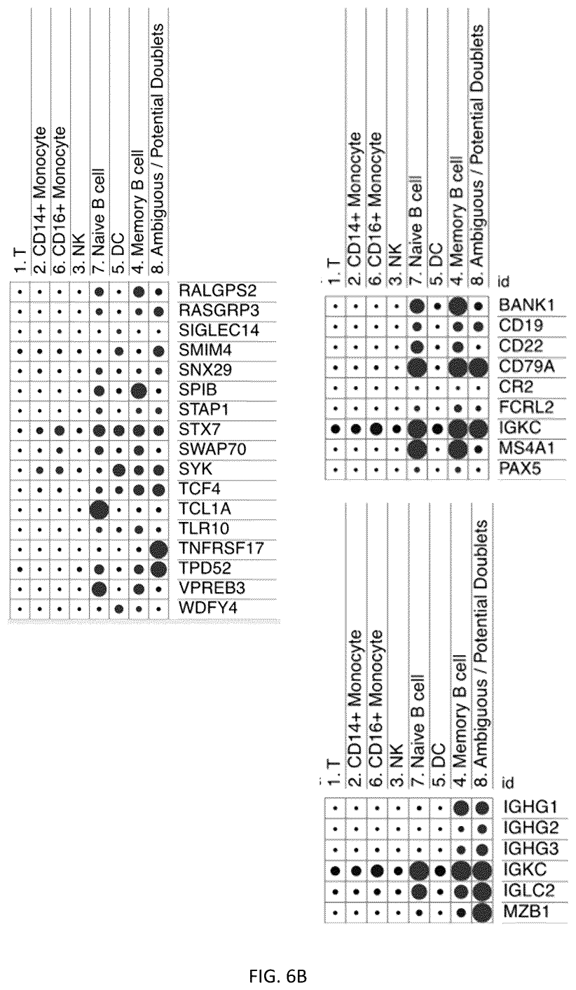

In view of the emerging understanding of B-cells being involved in regulating the outcome of immunotherapy given to cancer patients (US2020/123258A1), B-cell gene markers reported in the context of immunotherapy (as markers for determining B-cell infiltration in the tumor, not as individual gene biomarkers predictive for response to immunotherapy in bulk transcriptomic analyses) as mentioned in US2020/123258A1, US20190295720, WO2018209324, WO2020/205644 and WO2016/109546 were compiled and their power to predict T cell expansion in breast cancer (and thus of a positive response of a breast cancer patient to the immunotherapy) was first assessed, and the results were defined as AUC values of the individual genes as determined for the combined discovery and validation cohorts. Concurrently, expression of these genes was cross-checked in different cell types (depicted as heat map in FIGS. 4A and 4B), in different cell subtypes (depicted as heat map in FIGS. 5A and 5B) based on the current breast cancer transcriptome data, and as determined based on the Immgen database set relating to PBMCs of 2 healthy donors (https://singlecell.broadinstitute.org/single_cell/study/SCP345/ica-blood-mononuclear-cells-2-donors-2-sites; depicted in FIGS. 6A and 6B). Clearly, not all B-cell related genes retrieved from US2020/123258A1, US20190295720, WO2018209324, WO2020/205644 and WO2016/109546 (and listed in Table 3) are true B-cell specific genes. Based on the heatmaps and the Immgen database set, the genes that are predominantly expressed in B-cells (including plasma cells, naïve B cells, and memory B cells) are highlighted in grey in Table 5 with an asterisk. Independent thereof, only 11 out of the 99 genes assembled in Table 3 present with an AUC exceeding 0.8 based on the breast cancer bulk transcriptomne analysis: CD27 which is not B-cell specific; and FCRLS, IGHM, IGLC3, IGLL5, POU2AF1, IGKC, IGHG1, IGHG2, IGHG3, and IGLC2. Of the 10 B-cell specific genes, i) IGHM, IGLC3, IGLL5, IGKC, IGHG1, IGHG2, IGHG3, and IGLC2 are specific for plasma cells; and ii) only IGKC, IGHG1, IGHG2, IGHG3, and IGLC2 had an AUC exceeding 0.9 based on the breast cancer bulk transcriptome analysis.

TABLE 5
Evaluation of gene expression as predictor of T cell expansion
in breast cancer patients treated with ICB. Values are
given as AUC values determined for the combined discovery
and validation cohort (DC and VC, respectively).

| | AUC | | AUC | | AUC |
|---|---|---|---|---|---|
| | DC + | | DC + | | DC + |
| Gene | VC | Gene | VC | Gene | VC |
| ADAM28* | 0.67* | FCRLA* | 0.62* | STX7 | 0.65 |
| AFF3 | 0.75 | FGD2 | 0.57 | SWAP70* | 0.54* |
| AKAP2 | 0.53 | GPR18 | 0.65 | SYK | 0.58 |
| ALOX5 | 0.57 | HVCN1* | 0.62* | TCF4 | 0.71 |
| ARHGAP24* | 0.68* | IFT57* | 0.69* | TCL1A* | 0.67* |
| BLK* | 0.59* | IGHD* | 0.58* | TLR10* | 0.61* |
| BLNK | 0.49 | IGHM* | 0.88* | TNFRSF17* | 0.79* |
| BTK | 0.61 | IGLC3* | 0.87* | TPD52 | 0.50 |
| CCR6* | 0.69* | IGLL5* | 0.83* | VPREB3* | 0.64* |
| CD138/SDC1 | 0.44 | IRF8 | 0.62 | WDFY4* | 0.65* |
| CD24* | 0.63* | KIAA0125/ | 0.72 | IGHG1* | 0.93* |
| | | FAM30A | | | |
| CD27 | 0.83 | KIAA0226L/ | 0.59 | IGHG2* | 0.95* |
| | | RUBCNL | | | |
| CD3D | 0.76 | LAT2 | 0.75 | IGHG3* | 0.95* |
| CD3E | 0.76 | LY86 | 0.49 | IGLC2* | 0.92* |
| CD38 | 0.79 | LY9 | 0.61 | PAX5* | 0.71* |
| CD40 | 0.54 | MEF2C | 0.54 | MS4A1* | 0.65* |
| CD48 | 0.58 | MGAT5* | 0.66* | FCRL2* | 0.72* |
| CD72* | 0.65* | MICAL3 | 0.48 | CR2* | 0.58* |
| CD79B* | 0.62* | NCF1 | 0.68 | CD79A* | 0.72* |
| CD83 | 0.56 | ORAI2* | 0.55* | CD22* | 0.47* |
| CDCA7L | 0.47 | P2RX5* | 0.73* | CD19* | 0.73* |
| CHD7 | 0.61 | PDLIM1 | 0.66 | BANK1* | 0.69* |
| CIITA | 0.73 | PHACTR1 | 0.61 | IGKC* | 0.95* |
| CLEC17A* | 0.53* | PIK3AP1 | 0.67 | | |
| CNR2* | 0.57* | PIK3C2B | 0.50 | | |
| COBLL1 | 0.58 | PKIG* | 0.79* | | |
| CTSH | 0.56 | PLCG2* | 0.64* | | |
| CXCR5* | 0.69* | PLEKHF2* | 0.59* | | |
| CYB561A3 | 0.63 | PNOC | 0.73 | | |
| CYBB | 0.62 | POU2AF1* | 0.81* | | |
| DOK3 | 0.62 | POU2F2* | 0.68* | | |
| EAF2* | 0.78* | RAB30* | 0.55* | | |
| EBF1 | 0.75 | RABEP2* | 0.61* | | |
| FAM129C | 0.67 | RALGPS2* | 0.66* | | |
| FAM30A/KIAA0125* | 0.72* | RASGRP3 | 0.59 | | |
| FCER2* | 0.63* | SIGLEC14 | 0.63 | | |
| FCGR2B | 0.66 | SMIM4 | 0.52 | | |
| FCHSD2 | 0.62 | SNX29 | 0.55 | | |
| FCRL1* | 0.70* | SPIB* | 0.76* | | |
| FCRL5* | 0.83* | STAP1 | 0.68 | | |

### 2.3. Discussion

Over the last years, clinical studies have been investigating the potential of immune checkpoint blockade (ICB) in breast cancer (BC). Although these studies have shown promising results, this was only the case for subsets of patients and thus highlights the urgent need for biomarkers and a better understanding of underlying mechanisms to (non-) response. Moreover, since ICB is combined with neoadjuvant chemotherapy, it's difficult to discern predictive effects to ICB from chemotherapy. We therefore set up a window-of-opportunity study, including patients with early BC. The study consisted of two cohorts, a first discovery cohort (n=28) included treatment-naïve patients receiving one dose of pembrolizumab (anti-PD1) and a second validation cohort (n=11) included patients that received neoadjuvant chemotherapy prior to pembrolizumab. In both cohorts a biopsy was taken before anti-PD1 (“pre-treatment” biopsies) and ±9 days later a resection specimen (“on-treatment” biopsies) was taken during surgery. Paired pre- and on-treatment biopsies were subjected to single-cell RNA and T-cell receptor (TCR) sequencing. Because in this window-of-opportunity study no information about clinical response to anti-PD1 could be derived, we looked for a surrogate of response to anti-PD1. Since the number of expanded clonotypes upon treatment have been linked with response to ICB (Amaria et al. 2018, Nat Med 24:1649-1654; Tumeh et al. 2014, Nature 515:568-571), we used T-cell clonal expansion as a surrogate for response. Having scTCR-seq data we could identify clonotypes that expanded upon treatment. We used three definitions for determining expanded clonotypes and all three of them classified the same patients in to either patients with expansion (Es; n=8 for discovery cohort and n=3 for validation cohort) and patients with no or limited clonotype expansion (NEs; n=20 for discovery cohort and n=8 for validation cohort). Based on single-cell RNA, protein and TCR profiling we have shown multiple immune features, phenotypes and associated gene sets that negatively or positively correlate with T-cell expansion (Bassez et al. 2021, Nat Med 27:820-832). In conclusion, we present a map of the pre-treatment immune environment that is associated with T-cell expansion after neo-adjuvant anti-PD1 in patients with BC.

We here performed additional analyses to identify the minimal set of biomarkers that could predict response to anti-PD1. We performed two different analyses, one including TCR measures (i.e., clonality, richness and Gini index) and relative frequencies of cell (sub)types at baseline per patient. We identified a 6-feature signature as a minimal set of biomarkers that could reliably predict T-cell expansion status (Expanders versus Non-Expanders) with an overall accuracy of 100%. This signature consists of the relative frequency of T_{EX}-cells, TCR richness, Gini index, and relative frequencies of CD4 T_{EX}-cells, C7_CXCR1 macrophages and CD8 T_{EX}-cells.

A further analysis included the average expression of all genes in all pre-treatment cells per patient as input. A bulk-like approach was chosen for by defining the average expression on all cells, this because single-cell analyses are not (yet) feasible (in regards of time and cost) in clinical practice. As outcome of this further analysis, a set of 15 genes each individually predictive of T-cell expansion during subsequent immunotherapy (and thus predictive of a positive response to the immunotherapy). These 15 genes include five B-cell- (plasma cell-) specific genes (IGHG1, IGHG2, IGHG3, IGLC2 and IGKC). Previously, none of these 15 genes has been clearly and unambiguously linked to predicting response of breast cancer patients to immunotherapy. Moreover, the predictive power of each of these 15 genes in the breast cancer setting unexpectedly and significantly outperforms the predictive power of the same genes in the melanoma cancer setting. This finding is a proper warning against extrapolation of results obtained in one cancer to another cancer.

1. A method of detecting a subject having breast cancer as suitable for treatment with an immunotherapy or with an immunogenic therapy, the method comprising:
quantifying in a sample obtained from the subject prior to start, at start, or early after start of the immunotherapy or of the immunogenic therapy the expression level of at least one gene selected from the genes IGHG1, IGHG2, IGHG3, IGLC2, IGKC, HMGB2, MZB1, SEC11C, BATF, CXCL13, CXCR6, GZMB, PDCD1, TIGIT, and TNFRSF18;
detecting the expression level quantified for the selected gene as within a pre-determined range of expression level values of the gene wherein the pre-determined range of expression levels is indicative of a positive outcome of the immunotherapy or of the immunogenic therapy.

2. The method according to claim 1, wherein the at least one gene is selected from the genes IGHG1, IGHG2, IGHG3, IGLC2, and IGKC.

3. The method according to claim 1, wherein the at least one gene is selected from the genes IGHG1, IGHG2, IGHG3, IGLC2, IGKC, HMGB2, MZB1, SEC11C, BATF, CXCR6, PDCD1, TIGIT, and TNFRSF18.

4. The method according to a claim 1, further comprising:
quantifying one or more of the Gini-index of T-cell receptor clonotype distribution, the relative frequency of exhausted T-cells, the relative frequency of exhausted CD4+ T-cells, the relative frequency of exhausted CD8+ T-cells, the T-cell receptor richness, or the relative frequency of the C7_CX3CR1 subtype of T-cell inhibitory macrophages;
wherein high values within a pre-determined range of values of the Gini-index of T-cell receptor clonotype distribution, the relative frequency of exhausted T-cells, the relative frequency of exhausted CD4+ T-cells, and/or of the relative frequency of exhausted CD8+ T-cells are further indicative of a positive outcome of the immunotherapy or of the immunogenic therapy, and low values within a pre-determined range of values of the T-cell receptor richness, and/or of the relative frequency of the C7_CX3CR1 subtype of T-cell inhibitory macrophages are further indicative of a positive outcome of the immunotherapy or of the immunogenic therapy.

5. The method according to claim 1, wherein the quantification of the gene expression level is determined by transcriptome analysis and/or is determined by proteome analysis.

6. The method according to claim 5 wherein the transcriptome analysis is analysis of the bulk transcriptome.

7. The method according to a claim 1, further including detecting the status of one or more further diagnostic markers or biomarkers selected from immune checkpoint gene expression, markers of tumor mutational burden, T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES), expression of retrotransposons, infiltration of immune cells in the tumor of a subject having breast cancer, wherein the status of the one or more further diagnostic markers or biomarkers is further indicative of the positive outcome of the immunotherapy or of the immunogenic therapy.

8. The method according to claim 1, wherein the immunotherapy or immunogenic therapy is a therapy comprising an immune checkpoint blocker.

9. The method according to claim 1 wherein at least one data collecting step or at least one analysis step is performed by a computer system or via a computer program product.

10. (canceled)

11. (canceled)

12. (canceled)

13. (canceled)

14. A diagnostic kit comprising tools to detect the expression level of IGHG1, IGHG2, IGHG3, IGLC2, IGKC, HMGB2, MZB1, SEC11C, BATF, CXCL13, CXCR6, GZMB, PDCD1, TIGIT and TNFRSF18.

15. The diagnostic kit of claim 14 which is including the tools for detecting the status of at most 500 markers.

16. The method according to claim 1, wherein the method comprises treating the subject with an immunotherapy or with an immunogenic therapy.
